SEQUENCE LISTING (92 sequences)

SEQ ID NO: 1 - DNA, 30 bp, Artificial Sequence
  Description of Artificial Sequence Synthetic PCR primer
gcatcgtctcccatgagtgtgagaccttgc

SEQ ID NO: 2 - DNA, 19 bp, Artificial Sequence
  Description of Artificial Sequence Synthetic PCR primer
ctcgagctgcagggatccg

SEQ ID NO: 3 - DNA, 24 bp, Artificial Sequence
  Description of Artificial Sequence Synthetic PCR primer
gataatcccttttctaaactatac

SEQ ID NO: 4 - DNA, 27 bp, Artificial Sequence
  Description of Artificial Sequence Synthetic PCR primer
catttaaaaaattctatagatcagagg

SEQ ID NO: 5 - DNA, 33 bp, Artificial Sequence
  Description of Artificial Sequence Synthetic PCR primer
gaaaaattaagtgaaatcctaggaatgcttcac

SEQ ID NO: 6 - DNA, 10 bp, Human respiratory syncytial virus
ggggcaaata

SEQ ID NO: 7 - DNA, 48 bp, Artificial Sequence
  Description of Artificial Sequence Synthetic oligonucleotide
ggcctgataaatgcatagttacttaaaaagaggggcaaataaggtacc

SEQ ID NO: 8 - DNA, 47 bp, Artificial Sequence
  Description of Artificial Sequence Synthetic oligonucleotide
cgggtaccttatttgcccctctttttaagtaactatgcatttatcac

SEQ ID NO: 9 - protein, 20 aa, Human respiratory syncytial virus
  Strain A2
SARDGIRDAMIGLREEMIEK

SEQ ID NO: 10 - protein, 20 aa, Human respiratory syncytial virus
  Strain B1
SARDGIRDAMVGLREEMIEK

SEQ ID NO: 11 - protein, 20 aa, Ovine respiratory syncytial virus
SARDGIRDAMVGLREEMIEK

SEQ ID NO: 12 - protein, 20 aa, Bovine respiratory syncytial virus
AARDGIRDAMVGLREEMIEK

SEQ ID NO: 13 - protein, 20 aa, Avian pneumovirus
AARDGIRDAMIGMREELINS

SEQ ID NO: 14 - protein, 20 aa, Murine pneumonia virus
TARDEIRDALIGTREELIEM

SEQ ID NO: 15 - protein, 20 aa, Artificial Sequence
  Description of Artificial Sequence Synthetic peptide
SARDGIRDAMISLREEMIEK

SEQ ID NO: 16 - protein, 20 aa, Artificial Sequence
  Description of Artificial Sequence Synthetic peptide
SARDGIRDAMIGLREGMIEK

SEQ ID NO: 17 - protein, 20 aa, Artificial Sequence
  Description of Artificial Sequence Synthetic peptide
SARDGIRDAMISLREGMIEK

SEQ ID NO: 18 - protein, 20 aa, Artificial Sequence
  Description of Artificial Sequence Synthetic peptide
SARDGIRDAMIGLAAAMIEK

SEQ ID NO: 19 - protein, 194 aa, Human respiratory syncytial virus
  Strain A2
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 20 - protein, 176 aa, Murine pneumonia virus
MSVRPCKFEVQGFCSRGRNCKYSHKYWEWPLKTLMLRQNYMLNRIYRFLDTNTDAMSDVSGFDAPQRTAEYALGTIGVLKSYLEKTNNITKSIACGSLITVLQNLDVGLVIQARDSNTEDTNYLRSCNTILSYIDKIHKKRQIIHILKRLPVGVLCNLIQSVISIEEKINSSMKTE

SEQ ID NO: 21 - protein, 29 aa, Artificial Sequence
  Description of Artificial Sequence Synthetic peptide
MSVRPCKFEVRGHCSRGRNCKYSHKYWEW

SEQ ID NO: 22 - protein, 29 aa, Artificial Sequence
  Description of Artificial Sequence Synthetic peptide
MSVRPCKFEVQGFCLNGRNCKYSHKYWEW

SEQ ID NO: 23 - protein, 29 aa, Artificial Sequence
  Description of Artificial Sequence Synthetic peptide
MSVRPCKFEVQGFCSRGRNCKYSHNYWEW

SEQ ID NO: 24 - protein, 29 aa, Artificial Sequence
  Description of Artificial Sequence Synthetic peptide
MSVRPCKFEVRGFCSRGRNCKYSHKYWEW

SEQ ID NO: 25 - protein, 29 aa, Artificial Sequence
  Description of Artificial Sequence Synthetic peptide
MSVRPCKFEVQGHCSRGRNCKYSHKYWEW

SEQ ID NO: 26 - protein, 29 aa, Artificial Sequence
  Description of Artificial Sequence Synthetic peptide
MSVRPCKFEVQGFCLRGRNCKYSHKYWEW

SEQ ID NO: 27 - protein, 29 aa, Artificial Sequence
  Description of Artificial Sequence Synthetic peptide
MSVRPCKFEVQGFCSNGRNCKYSHKYWEW

SEQ ID NO: 28 - protein, 29 aa, Artificial Sequence
  Description of Artificial Sequence Synthetic peptide
MSVRPCKFEVRGFCLRGRNCKYSHKYWEW

SEQ ID NO: 29 - protein, 29 aa, Artificial Sequence
  Description of Artificial Sequence Synthetic peptide
MSVRPCKFEVRGFCSNGRNCKYSHKYWEW

SEQ ID NO: 30 - protein, 29 aa, Artificial Sequence
  Description of Artificial Sequence Synthetic peptide
MSRRPCKFEVQGFCSRGRNCKYSHKYWEW

SEQ ID NO: 31 - protein, 30 aa, Artificial Sequence
  Description of Artificial Sequence Synthetic polypeptide
MSVRNPCKFEVQGFCSRGRNCKYSHKYWEW

SEQ ID NO: 32 - protein, 29 aa, Artificial Sequence
  Description of Artificial Sequence Synthetic peptide
MSVRPCKFEVQGFCSRGRRCKYSHKYWEW

SEQ ID NO: 33 - protein, 29 aa, Artificial Sequence
  Description of Artificial Sequence Synthetic peptide
MSVRPCKFEVQGFCLRGRRCKYSHKYWEW

SEQ ID NO: 34 - protein, 30 aa, Artificial Sequence
  Description of Artificial Sequence Synthetic polypeptide
MSVRNPCKFEVQGFCLNGRNCKYSHKYWEW

SEQ ID NO: 35 - protein, 29 aa, Artificial Sequence
  Description of Artificial Sequence Synthetic peptide
MSRRPCKFEVQGFCLNGRNCKYSHKYWEW

SEQ ID NO: 36 - protein, 29 aa, Artificial Sequence
  Description of Artificial Sequence Synthetic peptide
MSVRPCKFEVRGFCLNGRNCKYSHKYWEW

SEQ ID NO: 37 - protein, 29 aa, Artificial Sequence
  Description of Artificial Sequence Synthetic peptide
MSVRPCKFEVQGFCLNGRRCKYSHKYWEW

SEQ ID NO: 38 - protein, 29 aa, Artificial Sequence
  Description of Artificial Sequence Synthetic peptide
MSVRPCKFEVRGHCLNGRNCKYSHKYWEW

SEQ ID NO: 39 - protein, 29 aa, Artificial Sequence
  Description of Artificial Sequence Synthetic peptide
MSVRPCKFEVRGHCLNGRRCKYSHKYWEW

SEQ ID NO: 40 - protein, 30 aa, Artificial Sequence
  Description of Artificial Sequence Synthetic polypeptide
MSRRNPCKFEIRGHCSNGKRCHFSHNYFEW

SEQ ID NO: 41 - protein, 30 aa, Artificial Sequence
  Description of Artificial Sequence Synthetic polypeptide
MSRRNPCKFEIRGHCLRGKRCHFSHNYFEW

SEQ ID NO: 42 - protein, 30 aa, Artificial Sequence
  Description of Artificial Sequence Synthetic polypeptide
MSRRNPCKFEIRGHCSRGKRCHFSHNYFEW

SEQ ID NO: 43 - protein, 30 aa, Artificial Sequence
  Description of Artificial Sequence Synthetic polypeptide
MSVRNPCKFEIRGHCLNGKRCHFSHNYFEW

SEQ ID NO: 44 - protein, 30 aa, Artificial Sequence
  Description of Artificial Sequence Synthetic polypeptide
MSRRNPCKFEIQGHCLNGKRCHFSHNYFEW

SEQ ID NO: 45 - protein, 30 aa, Artificial Sequence
  Description of Artificial Sequence Synthetic polypeptide
MSRRNPCKFEIRGFCLNGKRCHFSHNYFEW

SEQ ID NO: 46 - protein, 30 aa, Artificial Sequence
  Description of Artificial Sequence Synthetic polypeptide
MSRRNPCKFEIRGHCLNGKNCHFSHNYFEW

SEQ ID NO: 47 - protein, 30 aa, Artificial Sequence
  Description of Artificial Sequence Synthetic polypeptide
MSRRNPCKFEIQGFCLNGKRCHFSHNYFEW

SEQ ID NO: 48 - protein, 30 aa, Artificial Sequence
  Description of Artificial Sequence Synthetic polypeptide
MSRRNPCKFEIQGHCLNGKNCHFSHNYFEW

SEQ ID NO: 49 - protein, 30 aa, Artificial Sequence
  Description of Artificial Sequence Synthetic polypeptide
MSRRNPCKFEIRGFCLNGKNCHFSHNYFEW

SEQ ID NO: 50 - protein, 30 aa, Artificial Sequence
  Description of Artificial Sequence Synthetic polypeptide
MSRRNPCKFEIQGFCLNGKNCHFSHNYFEW

SEQ ID NO: 51 - DNA, 9 bp, Artificial Sequence
  Description of Artificial Sequence Synthetic oligonucleotide
gaaggtatg

SEQ ID NO: 52 - DNA, 9 bp, Artificial Sequence
  Description of Artificial Sequence Synthetic oligonucleotide
gaagatatg

SEQ ID NO: 53 - DNA, 9 bp, Human respiratory syncytial virus
  Strain A2
gaagaaatg

SEQ ID NO: 54 - protein, 16 aa, Human respiratory syncytial virus
  Strain A2
IETFDNNEEESSYSEE

SEQ ID NO: 55 - protein, 15 aa, Human respiratory syncytial virus
  Strain A2
LLEGNDSDNDLSLEF

SEQ ID NO: 56 - protein, 16 aa, Human respiratory syncytial virus
  Strain long
IETFDNNEEESSYSEE

SEQ ID NO: 57 - protein, 15 aa, Human respiratory syncytial virus
  Strain long
LLEGNDSDNDLSLEF

SEQ ID NO: 58 - protein, 16 aa, Human respiratory syncytial virus
  Strain B18537
IETFDNNEEESSYSEE

SEQ ID NO: 59 - protein, 15 aa, Human respiratory syncytial virus
  Strain B18537
LLEDNDSDNDLSLDF

SEQ ID NO: 60 - protein, 16 aa, Human metapneumovirus
LDLLDNEEEESSLTEE

SEQ ID NO: 61 - protein, 13 aa, Human metapneumovirus
IVEDESTSGESEE

SEQ ID NO: 62 - protein, 16 aa, Bovine respiratory syncytial virus
IETFDNNEEESSYSDE

SEQ ID NO: 63 - protein, 15 aa, Bovine respiratory syncytial virus
VLEDESSDNDLSLEF

SEQ ID NO: 64 - protein, 16 aa, Avian pneumovirus
LELLDNDDDESSLTEE

SEQ ID NO: 65 - protein, 13 aa, Avian pneumovirus
IVEDESTSGESEE

SEQ ID NO: 66 - protein, 16 aa, Ovine respiratory syncytial virus
IETFDNNEEESSYSDE

SEQ ID NO: 67 - protein, 15 aa, Ovine respiratory syncytial virus
ILEEDNSDNDLSLEF

SEQ ID NO: 68 - protein, 16 aa, Artificial Sequence
  Description of Artificial Sequence Synthetic peptide
IETFDNNEEELRYLEE

SEQ ID NO: 69 - protein, 15 aa, Artificial Sequence
  Description of Artificial Sequence Synthetic peptide
LLEGNDSDNDLSLEF

SEQ ID NO: 70 - protein, 16 aa, Artificial Sequence
  Description of Artificial Sequence Synthetic peptide
IETFDNNEEEDDYDEE

SEQ ID NO: 71 - protein, 15 aa, Artificial Sequence
  Description of Artificial Sequence Synthetic peptide
LLEGNDSDNDLSLEF

SEQ ID NO: 72 - protein, 16 aa, Artificial Sequence
  Description of Artificial Sequence Synthetic peptide
IETFDNNEEESSYSEE

SEQ ID NO: 73 - protein, 15 aa, Artificial Sequence
  Description of Artificial Sequence Synthetic peptide
LLEGNDDDNDLDLEF

SEQ ID NO: 74 - protein, 16 aa, Artificial Sequence
  Description of Artificial Sequence Synthetic peptide
IETFDNNEEESSYSEE

SEQ ID NO: 75 - protein, 15 aa, Artificial Sequence
  Description of Artificial Sequence Synthetic peptide
LLEGNDADNDLALEF

SEQ ID NO: 76 - protein, 16 aa, Artificial Sequence
  Description of Artificial Sequence Synthetic peptide
IETFDNNEEELRYLEE

SEQ ID NO: 77 - protein, 15 aa, Artificial Sequence
  Description of Artificial Sequence Synthetic peptide
LLEGNDADNDLALEF

SEQ ID NO: 78 - protein, 16 aa, Artificial Sequence
  Description of Artificial Sequence Synthetic peptide
IETFDNNEEELRYLEE

SEQ ID NO: 79 - protein, 15 aa, Artificial Sequence
  Description of Artificial Sequence Synthetic peptide
LLEGNDDDNDLDLEF

SEQ ID NO: 80 - protein, 6 aa, Human respiratory syncytial virus
  Strain A2
IGLREE

SEQ ID NO: 81 - protein, 6 aa, Artificial Sequence
  Description of Artificial Sequence Synthetic peptide
VGIKDD

SEQ ID NO: 82 - protein, 6 aa, Artificial Sequence
  Description of Artificial Sequence Synthetic peptide
IGVKDE

SEQ ID NO: 83 - protein, 241 aa, Human respiratory syncytial virus
  Strain A2
MEKFAPEFHGEDANNRATKFLESIKGKFTSPKDPKKKDSIISVNSIDIEVTKESPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMIGLREEMIEKIRTEALMTNDRLEAMARLRNEESEKMAKDTSDEVSLNPTSEKLNNLLEGNDSDNDLSLEDF

SEQ ID NO: 84 - protein, 90 aa, Human respiratory syncytial virus
  Strain A2
MTMPKIMILPDKYPCSITSILITSRCRVTMYNQKNTLCLNQNNPNNHMYSPNQTFNEIHWTSQELIDTIQNFLQHLGIIEDIYTIYILVS

SEQ ID NO: 85 - protein, 5 aa, Unknown
  Description of Unknown P protein phosphorylation Mutant 1
LRLSS

SEQ ID NO: 86 - protein, 5 aa, Unknown
  Description of Unknown P protein phosphorylation Mutant 2
DDDSS

SEQ ID NO: 87 - protein, 5 aa, Unknown
  Description of Unknown P protein phosphorylation Mutant 3
SSSDD

SEQ ID NO: 88 - protein, 5 aa, Unknown
  Description of Unknown P protein phosphorylation Mutant 4
SSSAA

SEQ ID NO: 89 - protein, 5 aa, Unknown
  Description of Unknown P protein phosphorylation Mutant 5
LRLAA

SEQ ID NO: 90 - protein, 5 aa, Unknown
  Description of Unknown P protein phosphorylation Mutant 6
LRLDD

SEQ ID NO: 91 - DNA, 6 bp, Artificial Sequence
  Description of Artificial Sequence Synthetic oligonucleotide
ggtacc

SEQ ID NO: 92 - DNA, 32 bp, Artificial Sequence
  Description of Artificial Sequence Synthetic oligonucleotide
agttacttaaaaagaggggcaaataaggtacc

(19) United States
(12) Patent Application Publication
(10) Pub. No.: US 2013/0266600 A1
(43) Pub. Date: Oct. 10, 2013

(54) Functional Mutations In Respiratory Syncytial Virus

(71) Applicant: Medimmune, LLC, Gaithersburg, MD (US)

(72) Inventors: Hong Jin, Mountain View, CA (US); Robert Brazas, Middleton, WI (US); Bin Lu, Los Altos, CA (US)

(21) Appl. No.: 13/742,539

(22) Filed: Jan. 16, 2013

Related U.S. Application Data
(63) Continuation of application No. 13/474,409, filed on May 17, 2012, now abandoned.
Application No. 13/474,409 is a continuation of application No. 12/537,288, filed on Aug. 7, 2009, now abandoned.
Application No. 12/537,288 is a continuation of application No. 12/241,229, filed on Sep. 30, 2008, now abandoned.
Application No. 12/241,229 is a continuation of application No. 10/672,302, filed on Sep. 26, 2003, now abandoned.
(60) Provisional application No. 60/444,287, filed on Jan. 31, 2003.
(60) Provisional application No. 60/414,614, filed on Sep. 27, 2002.

(51) Int. Cl. C12N 7/04

(52) U.S. Cl. CPC C12N 7/045
USPC 424/186.1; 435/236; 536/23.72; 435/320.1

(57) ABSTRACT

The present invention provides recombinant respiratory syncytial viruses that have an attenuated phenotype and that comprise one or more mutations in the viral P, M2-1 and/or M2-2 proteins, as well as live attenuated vaccines comprising such viruses and nucleic acids encoding such viruses. Recombinant RSV P, M2-1 and M2-2 proteins are described. Methods of producing attenuated recombinant RSV, and methods of quantitating neutralizing antibodies that utilize recombinant viruses of family Paramyxoviridae, are also provided.

### CROSS REFERENCE TO RELATED APPLICATIONS

This application is a non-provisional utility patent application claiming priority to and benefit of the following prior provisional patent applications: U.S. Ser. No. 60/414,614, filed Sep. 27, 2002, entitled “Functional Mutations in Respiratory Syncytial Virus” by Hong Jin, et al., and U.S. Ser. No. 60/444,287, filed Jan. 31, 2003, entitled “Functional Mutations in Respiratory Syncytial Virus” by Hong Jin! et al., each of which is incorporated herein by reference in its entirety for all purposes.

### STATEMENT AS TO RIGHTS TO INVENTIONS MADE UNDER FEDERALLY SPONSORED RESEARCH AND DEVELOPMENT

The invention was made with United States Government support under NIH SBIR grants 1R43A145267-01 and 2R44A145267-02. The United States Government may have certain rights in the invention.

### FIELD OF THE INVENTION

The present invention is in the field of vaccines against respiratory syncytial virus. The invention includes recombinant RSV having attenuated phenotypes, nucleic acids encoding such viruses, vaccines comprising such viruses, and methods of using such viruses to induce an immune response. Methods of producing attenuated RSV are also features of the invention, as are methods of determining antibody titers (e.g., an RSV neutralizing antibody titer).

### BACKGROUND OF THE INVENTION

Human respiratory syncytial virus is the leading cause of hospitalization for viral respiratory tract disease in infants and young children worldwide, as well as a significant source of morbidity and mortality in immunocompromised adults and in the elderly. To date, no vaccines have been approved which are able to prevent the diseases associated with RSV infection. RSV is classified in the Pneumovirus genus of the Paramyxoviridae family (Collins et al. (2001) Respiratory syncytial virus. pp. 1443-1483. In; Knipe & Howley (eds.) Fields Virology vol. 1, Lippincott, Williams & Wilkins, Philadelphia; Lamb & Kolakofsky (2001) Paramyxoviridae: the viruses and their replication. pp. 1305-1340. In; Knipe & Howley (eds.) Fields Virology vol. 1, Lippincott, Williams & Wilkins, Philadelphia). The RSV genome of A2 strain is 15,222 nt in length and contains 10 transcriptional units that encode 11 proteins (NS1, NS2, N, P, M, SH, G, F, M2-1, M2-2, and L). The genome is tightly bound by the N protein to form the nucleocapsid, which is the template for the viral RNA polymerase comprising the N, P and L proteins (Grosfeld et al. (1995) J. Virol. 69:5677-5686; Yu et al. (1995) J. Virol. 69:2412-2419). Each transcription unit is flanked by a highly conserved 10-nt gene-start (GS) signal, at which mRNA synthesis begins, and ends with a semiconserved 12- to 13-nt gene-end signal that directs polyadenylation and release of mRNAs (Harmon et al. (2001) J. Viro. 75:36-44; Kuo et al. (1996) J. Virol. 70:6892-6901). Transcription of RSV genes is sequential and there is a gradient of decreasing mRNA synthesis due to transcription attenuation (Barik (1992) J. Virol. 66:6813-6818; Dickens et al. (1984) J. Virol 52:364-369). The viral RNA polymerase must first terminate synthesis of the upstream message in order to initiate synthesis of the downstream mRNA.

The nucleocapsid protein (N), phosphoprotein (P), and large polymerase protein (L) constitute the minimal components for viral RNA replication and transcription in vitro (Grosfield et al. (1995) J. Virol 69:5677-5686; Yu et al. (1995) J. Virol. 69:2412-2419). The N protein associates with the genomic RNA to form the nucleocapsid, which serves as the template for RNA synthesis. The L protein is a multifunctional protein that contains RNA-dependent RNA polymerase catalytic motifs and is also probably responsible for capping and polyadenylation of viral mRNAs. However, the L protein alone is not sufficient for the polymerase function; the P protein is also required. Transcription and replication of RSV RNA are also modulated by the M2-1, M2-2, NS1, and NS2 proteins that are unique to the pneumoviruses. M2-1 is a transcription antitermination factor required for processive RNA synthesis and transcription read-through at gene junctions (Collins et al. (2001) in D. M. Knipe et al. (eds.), Fields Virology, 4^{th} ed. Lippincott, Philadelphia; Hardy et al. (1999) J. Virol 73:170-176; Hardy & Wertz (1998) J. Virol 72:520-526). M2-2 is involved in the switch between viral RNA transcription and replication (Bermingham & Collins (1999) Proc. Natl. Acad. Sci. USA 96:11259-11264; Jin et al. (2000) J. Virol 74:74-82). NS1 and NS2 have been shown to inhibit minigenome synthesis in vitro (Atreya et al. (1998) J. Virol 72:1452-1461).

The G and F proteins are the two major surface antigens that elicit anti-RSV neutralizing antibodies to provide protective immunity against RSV infection and reinfection. High levels of circulating antibodies correlate with protection against RSV infections or reduction of disease severity (Crowe (1999) Microbiol Immunol. 236:191-214). Two antigenic RSV subgroups have been recognized based on virus antigenic and sequence divergence (Anderson et al. (1985) J. Infect. Dis. 151:626-633; Mufson et al. (1985) J. Gen. Virol. 66:2111-2124). This antigenic diversity may be partly responsible for repeated RSV infection.

Efforts to produce a safe and effective RSV vaccine have focused on the administration of purified viral antigen or the development of live attenuated RSV for intranasal administration. For example, a formalin-inactivated virus vaccine not only failed to provide protection against RSV infection, but was shown to exacerbate symptoms during subsequent infection by the wild-type virus in infants (Kapikian et al., (1969) Am. J. Epidemiol. 89:405-421; Chin et al. (1969) Am. J. Epidemiol. 89:449-63). More recently, efforts have been aimed towards developing live attenuated temperature-sensitive mutants by chemical mutagenesis or cold passage of the wild-type RSV (Crowe et al., (1994) Vaccine 12:691-9). However, to date, these efforts have failed to produce a safe and effective vaccine. Virus candidates were either underattenuated or overattenuated (Kim et al., (1973) Pediatrics 52:56-63; Wright et al., (1976) J. Pediatrics 88:931-6) and some of the candidates were genetically unstable which resulted in the loss of the attenuated phenotype (Hodges et al. (1974) Proc Soc. Exp. Bio. Med. 145:1158-64).

Recently, a system for producing recombinant and chimeric viruses suitable for producing attenuated virus suitable for vaccine production has been described by the inventors and coworkers in WO 02/44334 by Jin et al., entitled “Recombinant RSV virus expression systems and vaccines,” the disclosure of which is incorporated herein in its entirety. The present invention provides additional species of attenuated and/or temperature sensitive RSV suitable for the production of live attenuated vaccines, as well as other benefits which will become apparent upon review of the disclosure.

### SUMMARY OF THE INVENTION

The present invention provides recombinant respiratory syncytial viruses (e.g., recombinant human respiratory syncytial viruses) that are genetically engineered to exhibit an attenuated phenotype. Such an attenuated recombinant respiratory syncytial virus (RSV) can be utilized as a live attenuated RSV vaccine. Recombinant viral proteins and nucleic acids encoding such recombinant proteins and/or recombinant viruses are also features of the invention.

Another aspect of the present invention provides methods for determining antibody titers (e.g., for quantitating neutralizing antibodies to subgroup A and/or subgroup B RSV or to another virus of family Paramyxoviridae). Compositions, recombinant viruses, and nucleic acids that relate to the methods are also features of the invention.

In one general class of embodiments, the invention provides a recombinant RSV that has an attenuated phenotype resulting from mutagenesis of a gene encoding the viral phosphoprotein (P) or a portion thereof. Thus, in one general class of embodiments, a recombinant RSV having an attenuated phenotype and comprising a phosphoprotein comprising at least one artificially mutated (e.g., substituted) amino acid residue is provided. For example, in one class of embodiments, the phosphoprotein comprises at least one mutated (e.g., substituted) amino acid residue at a position selected from the group consisting of position 172, position 174, position 175 and position 176. For example, the phosphoprotein can comprise a glycine to serine substitution at position 172 and/or a glutamic acid to glycine substitution at position 176. Another class of embodiments provides a recombinant RSV having an attenuated phenotype and comprising a phosphoprotein comprising a mutation (e.g., a deletion) of a plurality of amino acid residues selected from residues 172-176. For example, the phosphoprotein can comprise a deletion of residues 172-176 or a deletion of residues 161-180. A similar class of embodiments provides a recombinant RSV having an attenuated phenotype and comprising a phosphoprotein comprising a deletion of a plurality of amino acid residues selected from residues 236-241.

Yet another class of embodiments provides a recombinant RSV having an attenuated phenotype and comprising a phosphoprotein comprising at least one mutation (e.g., an amino acid substitution) that eliminates a phosphorylation site. For example, the phosphoprotein can comprise at least one substituted amino acid that replaces a serine, for example, the serine at position 116, 117, 119, 232, and/or 237. The serines can be mutated singly or in various combinations and each can, e.g., be substituted by any other residue (e.g., an alanine, an aspartic acid, an arginine, or a leucine).

A related class of embodiments provides methods, including methods for producing an attenuated RSV. The methods can, e.g., involve mutagenizing the RSV phosphoprotein (P) and/or nucleoprotein (N) and screening for decreased interaction between P and N (preferably, temperature sensitive decreased interaction). Mutations in P and/or N affecting the N-P interaction can then be introduced into an RSV genome or antigenome to produce an attenuated RSV. Thus, one aspect of the present invention provides methods of identifying a phosphoprotein or nucleoprotein having altered interaction with another protein. In the methods, a plurality of protein variants are provided, in which each protein variant comprises at least a portion of a first RSV protein. The first RSV protein is selected from the group consisting of an RSV phosphoprotein and an RSV nucleoprotein, and the portion of the first RSV protein typically comprises at least one artificial mutation (e.g., at least one mutated amino acid residue, e.g., one or more substituted, inserted or deleted amino acid residues). At least one candidate protein variant is identified that has an altered interaction with a second RSV protein or portion thereof (e.g., an RSV nucleoprotein or an RSV phosphoprotein).

In another general class of embodiments, the invention provides a recombinant RSV that has an attenuated phenotype resulting from mutagenesis of a gene encoding the viral M2-1 protein or a portion thereof. Thus, one class of embodiments provides a recombinant RSV having an attenuated phenotype and comprising an M2-1 protein comprising at least one artificially mutated (e.g., substituted or deleted) amino acid at an amino acid residue position selected from the group consisting of positions 3, 12, 14, 16, 17, and 20. For example, the M2-1 protein can comprise a leucine to serine substitution at position 16 and/or an asparagine to arginine substitution at position 17.

As another example, the M2-1 protein can be a chimera (e.g., of an RSV M2-1 protein and a PVM M2-1 protein). Thus, another class of embodiments provides a recombinant respiratory syncytial virus having an attenuated phenotype and comprising a chimeric M2-1 protein, which chimeric M2-1 protein comprises a plurality of residues from an RSV M2-1 protein and a plurality of residues from an M2-1 protein of another strain and/or species of virus (e.g., from a pneumonia virus of mice M2-1 protein). The chimeric protein can further comprise at least one mutated (e.g., substituted) amino acid

A related class of embodiments provides methods of identifying an M2-1 protein having an altered activity, including methods for producing an attenuated RSV. In the methods, one or more chimeric M2-1 proteins are provided, each of which comprises a plurality of residues from an RSV M2-1 protein from a first strain of virus and a plurality of residues from an M2-1 protein from a second strain of virus (e.g., a different strain of RSV or a different species of virus). At least one candidate chimeric M2-1 protein having an altered activity is identified; for example, by assaying M2-1-dependent processivity (e.g., in a minigenome assay), by assaying RNA binding by the candidate chimeric M2-1 protein (e.g., in a gel shift assay), and/or by assaying nucleoprotein binding by the candidate chimeric M2-1 protein (e.g., by coimmunoprecipitation). The activity of the M2-1 protein can be increased, or, typically, decreased. One or more mutations can be introduced into at least one of the candidate chimeric M2-1 proteins, and at least one mutated candidate chimeric M2-1 protein can be identified wherein the altered activity is further altered (typically, a decreased activity exhibited by the candidate chimeric M2-1 protein is further decreased for the mutated candidate chimeric M2-1 protein). At least one recombinant respiratory syncytial virus (RSV) whose genome or antigenome encodes at least one candidate chimeric or mutated candidate chimeric M2-1 protein can be produced and its replication assessed. If desired, mutations affecting the activity of the mutated candidate chimeric M2-1 protein can be introduced into an RSV M2-1 (i.e., a. non-chimeric RSV M2-1).

In another general class of embodiments, the invention provides a recombinant RSV that has an attenuated phenotype resulting from mutagenesis of a gene encoding the viral M2-2 protein or a portion thereof. Thus, one class of embodiments provides a recombinant RSV having an attenuated phenotype and comprising an M2-2 protein comprising at least one artificially mutated (e.g., substituted or deleted) amino acid. For example, the M2-2 can comprise a deletion of amino acid residues 1-2, 1-6, 1-8 or 1-10, or a deletion of the C-terminal 1, 2, 4, 8 or 18 amino acid residues. As another example, the M2-2 protein can comprise at least one artificially mutated (e.g., substituted) amino acid residue at position 2, position 4, position 5, position 6, position 11, position 12, position 15, position 25, position 27, position 34, position 41, position 56, position 58, position 66, position 75, position 80 and/or position 81.

Other embodiments provide a live attenuated RSV vaccine comprising an immunologically effective amount of a recombinant RSV of this invention, e.g., a vaccine comprising a recombinant RSV having one or more mutations in the P, M2-1 and/or M2-2 proteins as described herein. A related class of embodiments provides methods for stimulating the immune system of an individual to produce an immune response, preferably a protective immune response, against RSV by administering a recombinant attenuated RSV of this invention to the individual. Another class of embodiments provides a nucleic acid encoding a recombinant attenuated RSV and/or a mutant RSV phosphoprotein, M2-1 or M2-2 protein. For example, an RSV genome or antigenome encoding a recombinant attenuated RSV, e.g., one of those mentioned above, is a feature of the invention, as is a vector (e.g., a plasmid) comprising such a genome or antigenome.

In another aspect, the invention provides methods of determining an antibody titer (e.g., quantifying neutralizing antibodies to RSV or another virus of family Paramyxoviridae). In the methods, a sample comprising one or more antibodies and a recombinant virus whose genome or antigenome comprises a marker are contacted in the presence of cells in which the virus can replicate, which allows virus not neutralized by the antibodies to infect the cells. Replication of the virus is permitted, and the marker is detected. The cells can optionally be washed and lysed prior to detecting the marker (e.g., prior to quantitating expression of the marker). The virus comprises a respiratory syncytial virus (e.g., a human respiratory syncytial virus of subgroup A or subgroup B or a chimera thereof) or another virus belonging to the family Paramyxoviridae (e.g., a metapnuemovirus, a sendai virus, a parainfluenza virus, a mumps virus, a newcastle disease virus, a measles virus, a canine distemper virus, or a rinderpest virus). The marker can comprise one or more of, e.g., an optically detectable marker (e.g., a marker nucleic acid that encodes a beta galactosidase protein, a marker nucleic acid that encodes a green fluorescent protein, a marker nucleic acid that encodes a luciferase protein, or a marker nucleic acid that encodes a chloramphenicol transferase protein) or a selectable marker (e.g., an auxotrophic marker or a gene that confers cellular resistance to an antibiotic, e.g., a gene conferring resistance to neomycin). The sample comprising one or more antibodies can comprise, e.g., a serum, bronchial lavage or a nasal wash. The virus, the sample comprising the antibodies, and the cells can be combined in various orders. For example, the virus and the antibodies can be combined, and then the combined virus and antibodies can be combined with the cells. Other components (e.g., complement) can be used in the methods. For example, the virus, the sample comprising the antibodies, and complement can be combined, and then the combined virus, antibodies, and complement can be combined with the cells. The marker (e.g., expression of a marker protein encoded by the nucleic acid marker) can be detected by a number of methods known in the art. In some embodiments, expression of the marker is quantitated.

Compositions and recombinant viruses related to the methods provide additional features of the invention. Thus, one class of embodiments provides a composition comprising one or more antibodies and a recombinant virus that belongs to the family Paramyxoviridae and whose genome or antigenome comprises a marker. The virus can comprise a respiratory syncytial virus (e.g., a human respiratory syncytial virus of subgroup A or subgroup B or a chimera thereof) or another virus belonging to the family Paramyxoviridae (e.g., a metapneumovirus, a sendai virus, a parainfluenza virus, a mumps virus, a newcastle disease virus, a measles virus, a canine distemper virus, or a rinderpest virus). The marker can comprise one or more of, e.g., an optically detectable marker (e.g., a marker nucleic acid that encodes a beta galactosidase protein, a marker nucleic acid that encodes a green fluorescent protein, a marker nucleic acid that encodes a luciferase protein, a marker nucleic acid that encodes a chloramphenicol transferase protein) or a selectable marker (e.g., an auxotrophic marker or a gene that confers cellular resistance to an antibiotic, e.g., a gene conferring resistance to neomycin).

Another class of embodiments provides a recombinant respiratory syncytial virus (RSV) comprising a genome or antigenome that comprises a marker, which marker comprises one or more of: a marker nucleic acid that encodes a beta galactosidase protein, a marker nucleic acid that encodes a luciferase protein, or a marker nucleic acid that encodes a selectable marker protein (e.g., a gene that confers cellular resistance to an antibiotic, e.g., a gene conferring resistance to neomycin). Yet another class of embodiments provides a recombinant virus of family Paramyxoviridae. The recombinant virus comprises a metapneumovirus, a sendai virus, a parainfluenza virus, a mumps virus, or a canine distemper virus. The recombinant virus comprises a genome or antigenome comprising a marker, for example, one or more of: a nucleic acid that encodes an optically detectable marker protein (e.g., a marker nucleic acid that encodes a beta galactosidase protein, a marker nucleic acid that encodes a green fluorescent protein, a marker nucleic acid that encodes a luciferase protein, or a marker nucleic acid that encodes a chloramphenicol transferase protein) or a marker nucleic acid that encodes a selectable marker protein (e.g., a gene that confers cellular resistance to an antibiotic, e.g., a gene conferring resistance to neomycin). A related class of embodiments provides a nucleic acid encoding such a recombinant RSV or virus of family Paramyxoviridae.

### BRIEF DESCRIPTION OF THE DRAWINGS

FIG. 1: Sequence alignment of the P proteins from residues 161 to 180, illustrating charged residue rich region flanking positions 172-176, of various pneumoviruses: RSV-A2, human RSV subgroup A2 (SEQ ID NO:9); RSV-B1, human RSV subgroup B1 (SEQ ID NO:10); ORSV, ovine RSV (SEQ ID NO:11); BRSV, bovine RSV (SEQ ID NO:12); APV, avian pneumovirus (SEQ ID NO:13); and PVM, pneumonia virus of mice (SEQ ID NO: 14). Also shown are the following mutants: G172S, Gly replaced by Ser at position 172 (SEQ ID NO:15); E176G, Glu replaced by Gly at position 176 (SEQ ID NO:16); G172S/E176G, double mutant containing both G172S and E176G (SEQ ID NO:17); 174-176A, three consecutive charged residues from positions 174 to 176 replaced by Ala (SEQ ID NO:18); Δ161-180, an internal deletion from residues 161 to 180; and ΔC6, a C-terminal deletion from residues 236 to 241.

FIG. 2: A. Sequence alignment of the RSV A2 M2-1 protein (A2; SEQ ID NO:19) and the pneumonia virus of mice M2-1 protein (PVM; SEQ ID NO:20). The conserved Cys₃-His₁ motif is indicated. B. Line graph illustrating relative activity of RSV and PVM M2-1 proteins in an RSVlacZ minigenome assay.

FIG. 3: A. Schematic illustration of RP and PR M2-1 chimeric proteins in comparison with RSV (white with black dots) and PVM (black with white dots) M2-1. B. Line graph illustrating relative activity of RP and PR chimeric M2-1 proteins in an RSVlacZ minigenome assay.

FIG. 4: A. Sequence alignment of M2-1 N-terminal mutants, showing the residues that were changed from PVM to RSV for each PR M2-1 mutant PR1-PR19. PR1 SEQ ID NO:21; PR2, SEQ ID NO:22; PR3, SEQ ID NO:23; PR4, SEQ ID NO:24; PR5, SEQ ID NO:25; PR6, SEQ ID NO:26; PR7, SEQ ID NO:27; PR8, SEQ ID NO:28; PR9, SEQ ID NO:29; PR10, SEQ ID NO:30; PR11, SEQ ID NO:31; PR12, SEQ ID NO:32; PR13, SEQ ID NO:33; PR14, SEQ ID NO:34; PR15, SEQ ID NO:35; PR 16, SEQ ID NO:36; PR17, SEQ ID NO:37; PR18, SEQ ID NO:38; PR19, SEQ ID NO:39, B. Bar graph illustrating relative activity in an RSV lacZ minigenome assay; the level of β-galactosidase expressed by each mutant is normalized to RSV M2-1.

FIG. 5: A. Sequence alignment of M2-1 N-terminal mutants, showing the residues that were changed from RSV to PVM for each RSV M2-1 mutant RS1-RS11. RS1, SEQ ID NO:40; RS2, SEQ ID NO:41; RS3, SEQ ID NO:42; RS4, SEQ ID NO:43; RS5, SEQ ID NO:44; RS6, SEQ ID NO:45; RS7, SEQ ID NO:46; RS8, SEQ ID NO:47; RS9, SEQ ID NO:48; RS10, SEQ ID NO:49; RS11, SEQ ID NO:50. B. Bar graph illustrating relative activity of M2-1 mutants; the level of β-galactosidase expressed by each mutant is normalized to wt RSV M2-1.

FIG. 6: A. Northern blot illustrating relative expression levels of lacZ and M2-1 in M2-1 mutants. B. Coimmunoprecipitation of RNA from radiolabeled cells with anti-M2-1 monoclonal antibodies.

FIG. 7: A. Co-immunoprecipitation of N and M2-1 proteins from radiolabeled cells with anti-M2-1 monoclonal antibody B. Co-immunoprecipitation of N and M2-1 proteins from radiolabeled cells with anti-RSV antibody.

FIG. 8: Immunoprecipitation analysis of N-P interaction in cells transiently expressing N and P proteins.

FIG. 9: Bar graph illustrating relative activity level of P protein mutants in minigenome assay. Insert illustrates N and P protein expression levels by Western analysis.

FIG. 10: Photomicrographs illustrating plaque formation at different temperatures.

FIG. 11: Line graphs illustrating growth kinetics of rA2-P172 and rA2-P176 mutants.

FIG. 12: Immunoprecipitation of viral proteins from wild-type and mutant RSV-infected cells.

FIG. 13: A. Sequence analysis illustrating reversion of rA2-P176 during passage. Sequence of the P gene in the region of residue 176, from rA2-P176 (SEQ ID NO:51), from revertant E176D (SEQ ID NO:52), and from wt (SEQ ID NO:53). B. Bar graph-illustrating growth of E176D revertant at various temperatures.

FIG. 14: Sequence alignment of P proteins in the central region (nt 106-121) and in the C terminal region (nt 226-241). The serine residues in these regions are underlined. P proteins illustrated are the P proteins from: RSV-A2, human RSV subgroup A2 strain (central, SEQ ID NO:54; C-terminal, SEQ ID NO:55); Long, human RSV subgroup A long strain (central, SEQ ID NO:56; C-terminal, SEQ ID NO:57); B18537, Human RSV subgroup B strain 18537 (central, SEQ ID NO.58; C-terminal, SEQ ID NO: 59); MPV, human metapneumovirus (central, SEQ ID NO:60; C-terminal, SEQ ID NO:61); Bovine, bovine RSV (central, SEQ ID NO.62; C-terminal, SEQ ID NO:63); Avian, avian Pneumovirus (central, SEQ ID NO:64; C-terminal, SEQ ID NO:65); and Ovine, ovine RSV (central, SEQ ID NO:66; C-terminal, SEQ ID NO: 67). P protein mutants Mut1-Mut6 are also depicted. Mut1 (central, SEQ ID NO:68; C-terminal, SEQ ID NO:69), Mut2 (central, SEQ ID NO.70; C-terminal, SEQ ID NO:71), Mut3 (central, SEQ ID NO:72; C-terminal, SEQ ID NO:73), Mut4 (central, SEQ ID NO: 74; C-terminal, SEQ ID NO:75), Mut5 (central, SEQ ID NO:76; C-terminal, SEQ ID NO:77), Mut6 (central, SEQ ID NO:78; C-terminal, SEQ ID NO:79).

FIG. 15: Functional analysis of RSV P protein phosphorylation mutants. A. Bar graph illustrating relative transcriptional activity of mutants lacking phosphorylation sites at positions 116, 117, 119, 232 and/or 237. B. Bar graph illustrating relative activity of mutants in the presence of wild-type P protein. C. Northern analysis of transcription and replication of RSVCAT/EGFP reporter minigenome in cells expressing mutant P proteins lacking one or more phosphorylation sites.

FIG. 16: Line graphs illustrating relative growth kinetics of P phosphorylation site mutant RSV rA2-PP2 and rA2-PP5.

FIG. 17: Bar graphs illustrating relative proportion of cell associated virus for various phosphorylation mutants.

FIG. 18: Immunoprecipitation of RSV-infected cells infected with wild-type or phosphorylation mutants.

FIG. 19: A. Northern analysis of expression levels of genomic or P protein RNA in cells infected with phosphorylation mutants. B. Western analysis illustrating relative expression levels of RSV proteins detected with polyclonal RSV antibodies.

FIG. 20: Schematic illustration of RSV-lacZ constructs.

FIG. 21: A. Line graphs illustrating replication of recombinant lacZ viruses in Vero cells. B. Line graphs illustrating replication of recombinant lacZ viruses in HEp-2 cells.

FIG. 22: A. Western analysis of β-galactosidase expression in A-lacZ and B-lacZ infected cells using anti-β-galactosidase antibody. B. Line graphs illustrating relative β-galactosidase activity in A-lacZ and B-lacZ infected cells.

FIG. 23: A. Line graph illustrating detection of neutralizing anti-RSV antibodies by microneutralization assay. B. Western analysis of infected cells with adult human serum and RSV-infected monkey serum.

FIG. 24: A. Sequence of the phosphoprotein (P) of human RSV strain A2 (SEQ ID NO:83, Genbank ID 74915). B. Sequence of the M2-2 protein of human RSV strain A2 (SEQ ID NO:84).

FIG. 25: A. Schematic illustrating the positions of potential start codons in wild-type M2-2 and three mutants (M2-A1, M2-A2 and M2-A3). B. Line graph illustrating in vitro activity of M2-2 initiation codon mutants.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a, ” “an” and “the” include plural referents unless the content clearly dictates otherwise. Thus, for example, reference to “a virus” includes a plurality of viruses; reference to a “host cell” includes mixtures of host cells, and the like. In describing and claiming the present invention, the following terminology will be used in accordance with the definitions set out below.

The terms “nucleic acid,” “polynucleotide,” “polynucleotide sequence” and “nucleic acid sequence” refer to single-stranded or double-stranded deoxyribonucleotide or ribonucleotide polymers, or chimeras or analogs thereof. As used herein, the term optionally includes polymers of analogs of naturally occurring nucleotides having the essential nature of natural nucleotides in that they hybridize to single-stranded nucleic acids in a manner similar to naturally occurring nucleotides (e.g., peptide nucleic acids). Unless otherwise indicated, a particular nucleic acid sequence of this invention encompasses complementary sequences, in addition to the sequence explicitly indicated.

The term “gene” is used broadly to refer to any nucleic acid associated with a biological function. Thus, genes include coding sequences and/or the regulatory sequences required for their expression. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include “promoters” and “enhancers, ” to which regulatory proteins such as transcription factors bind, resulting in transcription of adjacent or nearby sequences. A “tissue specific” promoter or enhancer is one which regulates transcription in a specific tissue type or cell type, or types.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not autonomously replicating. Most commonly, the vectors of the present invention are plasmids.

An “expression vector” is a vector, such as a plasmid, which is capable of promoting expression as well as replication of a nucleic acid incorporated therein. Typically, the nucleic acid to be expressed is “operably linked” to a promoter and/or enhancer, and is subject to transcription regulatory control by the promoter and/or enhancer.

In the context of the invention, the term “isolated” refers to a biological material, such as a nucleic acid or a protein, which is substantially free from components that normally accompany or interact with it in its naturally occurring environment. The isolated material optionally comprises material not found with the material in its natural environment, e.g., a cell. For example, if the material is in its natural environment, such as a cell, the material has been placed at a location in the cell (e.g., genome or genetic element) not native to a material found in that environment. For example, a naturally occurring nucleic acid (e.g., a coding sequence, a promoter, an enhancer, etc.) becomes isolated if it is introduced by non-naturally occurring means to a locus of the genome (e.g., a vector, such as a plasmid or virus vector, or amplicon) not native to that nucleic acid. Such nucleic acids are also referred to as “heterologous” nucleic acids. An isolated virus, for example, is in an environment (e.g., a cell culture system, or purified from cell culture) other than the native environment of wild-type-virus (e.g., the nasopharynx of an infected individual).

The term “recombinant” indicates that the material (e.g., a virus, a nucleic acid or a protein) has been artificially or synthetically (non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other procedures, or by chemical or other mutagenesis. For example, when referring to a virus, e.g., a respiratory syncytial virus, the virus is recombinant when it is produced by the expression of a recombinant nucleic acid.

An “artificial mutation” is a mutation introduced by human intervention. Thus, an “artificially mutated” amino acid residue is a residue that has been mutated as a result of human intervention, and an “artificial conservative variation” is a conservative variation that has been produced by human intervention. For example, a wild-type virus (e.g., one circulating naturally among human hosts) or other parental strain of virus can be “artificially mutated” using recombinant DNA techniques to alter the viral genome (e.g., the viral genome can be altered by in vitro mutagenesis, or by exposing it to a chemical, ionizing radiation, or the like and then performing in vitro or in vivo selection for a temperature sensitive, cold sensitive, or otherwise attenuated strain of virus).

The term “chimeric” or “chimera,” when referring to a virus, indicates that the virus includes genetic and/or polypeptide components derived from more than one parental viral strain or source. Similarly, the term “chimeric” or “chimera,” when referring to a viral protein, indicates that the protein includes polypeptide components derived from more than one parental viral strain or source.

The term “introduced” when referring to a heterologous or isolated nucleic acid refers to the incorporation of a nucleic acid into a eukaryotic or prokaryotic cell where the nucleic acid can be incorporated into the genome of the cell (e.g., chromosome, plasmid, plastid or mitochondrial DNA), converted into an autonomous replicon, or transiently expressed (e.g., transfected mRNA). The term includes such methods as “infection,” “transaction,” “transformation” and “transduction.” In the context of the invention a variety of methods can be employed to introduce nucleic acids into prokaryotic cells, including electroporation, calcium phosphate precipitation, lipid mediated transfection (lipofection), etc.

The term “host cell” means a cell which contains a heterologous nucleic acid, such as a vector, and supports the replication and/or expression of the nucleic acid. Host cells can be prokaryotic cells such as E. coli, or eukaryotic cells such as yeast, insect, amphibian, avian or mammalian cells, including human cells. Exemplary host cells in the context of the invention include HEp-2 cells, CEK cells and Vero cells.

An “antigenome” is a single-stranded nucleic acid that is complementary to a single-stranded viral (e.g., RSV) genome.

An RSV “having an attenuated phenotype” or an “attenuated” RSV exhibits a substantially lower degree of virulence as compared to a wild-type virus (e.g., one circulating naturally among human hosts). An attenuated RSV typically exhibits a slower growth rate and/or a reduced level Of replication (e.g., a peak titer, e.g., in cell culture, in a human vacinee's nasopharynx or in an animal model of infection, that is at least about ten fold, preferably at least about one hundred fold, less than that of a wild-type RSV).

An “Immunologically effective amount” of RSV is an amount sufficient to enhance an individual's (e.g., a human's) own immune response against a subsequent exposure to RSV. Levels of induced immunity can be monitored, e.g., by measuring amounts of neutralizing secretory and/or serum antibodies, e.g., by plaque neutralization, complement fixation, enzyme-linked immunosorbent, or microneutralization assay.

A “protective immune response” against RSV refers to an immune response exhibited by an individual (e.g., a human) that is protective against serious lower respiratory tract disease (e.g., pneumonia and/or bronchiolitis) when the individual is subsequently exposed to and/or infected with wild-type RSV. In some instances, the wild-type (e.g., naturally circulating) RSV can still cause infection, particularly in the upper respiratory tract (e.g., rhinitis), but it can not cause a serious infection. Typically, the protective immune response results in detectable levels of host engendered serum and secretary antibodies that are capable of neutralizing virus of the same strain and/or subgroup (and possibly also of a different, non-vaccine strain and/or subgroup) in vitro and in vivo.

### DETAILED DESCRIPTION

Conditional lethal mutations, e.g., are important for the development of live attenuated vaccines. The temperature-sensitive lesions previously identified in chemically mutagenized or cold-passaged RSV have mostly been mapped to the L protein (Crowe et al. (1996) Virus Genes 13:269-273; Juhasz et al. (1997) J. Virol. 71:5814-5819; Tolley et al. (1996) Vaccine 14:1637-1646; Whitehead et al. (1998) Virology 247:232-239), possibly due to its large size. Production of deletion mutants in a recombinant system by the inventors and their coworkers has been successfully used to generate mutant RSV with an attenuated phenotype (WO 02/44334). The present invention relates to the identification of independent mutations which confer attenuated and/or temperature sensitive phenotypes important in the production of live attenuated virus vaccines.

### Functional Mutations in the RSV P Protein

### Mutations in RSV P that Confer Temperature Sensitivity

The phosphoprotein (P protein) of human Respiratory Syncytial Virus (RSV) is an essential component of the viral RNA polymerase, along with the large polymerase (L) and nucleocapsid (N) proteins (Grosfeld et al. (1995) J. Virol. 69:5677-5686; Yu et al. (1995) J. Virol. 69:2412-2419). Interaction of the RSV P protein with the N and L proteins promotes the formation of a transcriptase complex that is essential for viral RNA transcription and replication (Garcia-Barreno et al. (1996) J. Virol. 70:901-808; Khattar et al. (2001) Virology 285:253-269; Khattar et al. (2001) J. Gen Virol. 82:775-779). Although the L protein is the catalytic RNA polymerase, the P protein is essential for transcription and replication of viral RNA (Curran et al. (1991) EMBO J. 10:3079-3085; Horikami et. al. (1992) J. Virol. 66:4901-4908). In addition to the N, P and L proteins, several other viral proteins are required for RSV RNA synthesis. The antitermination function of M2-1 is essential for processive RNA synthesis and suppression of transcription termination in intergenic regions (Collins et al. (1995) Proc. Natl. Acad. Sci. USA 92:11563-11567; Hardy & Wertz (2000) J. Virol. 74:5880-5885). M2-2 has been postulated to have a role in regulating the switch between viral RNA transcription and replication processes (Bermingham & Collins Proc. Natl. Acad. Sci. USA 96; 11259-11264; Jin et al. (2000) J. Virol, 74:74-82).

The RSV subgroup A P protein is 241 amino acids in length, which is much shorter than the P proteins of other paramyxoviruses. Although the RSV P protein shares no sequence homology with the P proteins of other paramyxoviruses, it shares similar structure and function in viral replication, and forms homotetramers (Assenjo Villanueva (2000) FEBS Lett. 467:279-284), similar to the Sendai virus P protein (Tarbouriech et al. (2000) Virology 266:99-109; Villanueva et al. (2000) Nat. Struct. Biol 7:777-781). The interaction of the N and P proteins enables proper folding of N protein and enables N protein to encapsidate viral RNA during RNA replication (Bowman et al. (1999) J. Virol 73:6474-6483; Huber et al. (1991) Virology 185:299-308; Masters & Banerjee (1988) J. Virol. 62:2658-2664). By analogy with the other paramyxovirus P proteins, the P protein of RSV likely acts as a cofactor that serves both to stabilize the L protein and to place the polymerase complex on the N protein-RNA template.

Although the C-terminal six amino acids of the P protein have been shown to play a major role in binding to the N protein (Garcia Barreno et al. (1996) J. Virol. 70:801-808; Slack & Easton (1998) Virus Research 55:167-176), other regions in the P protein are also likely to be important for the formation of the N-P complex. For example, deletion mutants lacking the N-terminal 10 amino acids failed to induce coaggregation of N in coprecipitation experiments (Garcia Barreno et al. (1996) J. Virol. 70:801-808). Studies of the P protein of bovine RSV have shown that in addition to the C-terminal end and an internal region between residues 161 to 180 are required for N-P complex formation as assayed by coimmunoprecipitation. (Mallipeddi et al. (1996) J. Gen Virol 77; 1019-1023; Khattar et al. (2001) J. Gen Virol. 82:775-779).

The present invention identifies mutations in the P protein that confer a temperature-sensitive (ts) phenotype on recombinant RSV. These variants were isolated by assaying a randomly mutagenized P gene cDNA library using a yeast two-hybrid system for mutations that confer a temperature-sensitive N-P interaction (Lu et al. (2002) J. Virol. 76:2871-2880). Two independent P mutations, one at residue 172 and the other at 176, were identified that resulted in a temperature-sensitive interaction with N. Both mutants were assayed in a minigenome replicon system and in a whole virus system by introducing the mutations into recombinant RSV using reverse genetics (Collins et al. (1995) Proc. Natl. Acad. Sci. 92:11563-11567; Jin et al. (1998) Virology 251:206-214).

Amino acid substitutions of serine for glycine at position 172 (G172S) and of glycine for glutamic acid at position 176 (E176G) affect the N-P interaction in a temperature-dependent manner. The replication of recombinant viruses bearing either the G172S or the E176G mutation exhibits a ts phenotype in tissue culture. Coincidentally, the G172S mutation coincides with the ts mutation identified in the RSV subgroup B RSN-2 strain (Caravokyri et al. (1992) J. Gen Virol. 73:865-873; Faulkner et al. (1976) J. Virol. 20:487-500). Introduction of a G172S mutation into the P gene of the RSV subgroup A RSS-2 strain also results in much-reduced replication of an RSV minigenome at 37 and 39° C. (Marriott et al. (1999) J. Virol, 73:5162-5165).

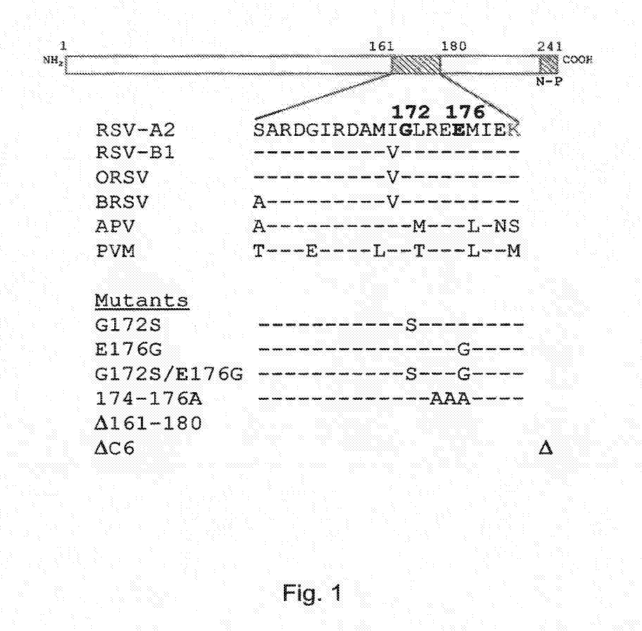

The E176G mutation exhibits a more severe effect on the P protein function than the G172S mutation. For example, recombinant rA2-P176 virus is more temperature sensitive in tissue culture and more restricted in replication in the respiratory tracts of mice and cotton rats than recombinant rA2-P172 virus. The region flanking 172 to 176 is rich in charged residues, and is highly conserved among different pneumoviruses (FIG. 1). Alteration of the charged residues at positions 174-176 to alanine produces a nonfunctional protein in a minigenome system, indicating a critical role of these charged residues. Introduction of both the G172S and E176G mutations in the P gene resulted in a synergistic effect that completely abolished the P protein function in the minigenome assay, and virus was not recovered from the cDNA bearing a combination of these two mutations.

Recombinant virus rA2-P176 rapidly reverts (e.g., undergoes amino acid substitutions) when the virus-infected cells are incubated at 37° C., leading to the loss of the ts phenotype. Reversions to wild-type (wt) are infrequent, most likely because Gly (GGT) contains two nucleotide changes compared to Glu (GAA). Rather, the introduced Gly is predominantly changed to Asp (GAT), also a negatively charged residue, as well as Cys and Ser, which are able to interact with other protein residues through disulfide or hydrogen bonds, respectively, suggesting that a charged residue at position 176 is important in maintaining temperature stability of the P protein. When assayed in a CAT minigenome expression assay, the P-E176D expressing cells have CAT expression approximately 50% of that of the wt, much higher than the 5% activity of E176G. Similarly, replacement of E176 with Ala did not significantly reduce the P protein function in a minigenome assay.

G172S and E176G mutations also result in temperature sensitive alterations in the interactions between P and N in yeast. While the function of each mutant was only slightly reduced at 33° C., the function was greatly reduced at 37° C., and was further reduced at 39° C. The expression level of G172S and E176G protein in transfected cells at 37 and 39° C. is similar to that of wt P, indicating that the temperature sensitivity is not due to the thermolability of the protein. At 37° C. cells infected with rA2-P172 or rA2-P176 exhibit a reduced N-P interaction, as demonstrated by a two-fold or greater reduction in N protein coimmunoprecipitated with the P protein. The reduced ability of G172S and E176G mutations to interact with N is likely to explain the ts phenotype of viruses having these mutations.

Additionally, human RSV P protein with a deletion of amino acid residues 161 to 180 coimmunoprecipitates with N, although does not function in the RSV minigenome replication assay.

### Mutations in the Phosphorylation Sites of RSV P

RSV P protein is constitutively phosphorylated within the virion core as well as in infected cells. Phosphorylation is mediated by the cellular casein kinase II (Dupuy et al. (1999) J. Virol. 73:8384-8392; Villanueva et al. (1994) J. Gen. Virol. 75:555-565) J. Gen Virol. 75:555-565) on two clusters of serines: 116, 117, and 119 (116/117/119) in the central region and 232 and 237 (232/237) in the C-terminal region (Navarro et al. (1991) J. Gen. Virol. 72:1455-1459; Sanchez-Seco et al. (1995) J. Gen. Virol. 76:425-430; Villanueva et al. (2000) J. Gen. Virol. 81:129-133; Villanueva et al. (1994) J. Gen. Virol. 75:555-565). Approximately 80% of P protein phosphorylation is localized to Ser 232 and the remaining 20% is distributed among the serines at positions 116, 117, 119, and 237.

Bacterially expressed, nonphosphorylated P protein cannot form tetramers (Assenjo & Villanueva (2000) FEBS Lett. 467:279-284) required to support transcription in an in vitro system (Batik et al. (1995) Virology 213:405-412). Phosphorylation of bacterially expressed P protein restores its ability to support transcription, suggesting that the phosphorylated P protein is required to convert the newly initiated polymerase into a stable complex. In contrast to these observations, inhibition of phosphorylation in RSV-infected cells does not abolish viral transcription or replication (Barik et al. (1995) Virology 213:405-412, Villanueva et al. (1994) J. Gen Virol. 101-108), nor is the bulk of P protein phosphorylation required for RNA synthesis in an RSV minigenome system (Villanueva et al. (2000) J. Gen Virol. 81:129-133). In addition, substitutions of S232 or S237 by alanine do not prevent interaction with N protein, as shown by the formation of inclusion bodies in cotransfected cells (Garcia-Barreno et al. (1996) J. Virol. 70:801-808) and reduction of phosphorylation by phosphorylation inhibitors did not impact tetramer formation of P protein (Bowman et al. (1999) J. Virol. 73:6474-6483). P protein phosphorylation adds a negative charge to the polypeptide via the phosphate group. It has been shown previously that removal of the phosphate group from Ser232 of P protein halted transcription elongation in vitro, but substitution of Ser232 by aspartic acid restored transcription activity to 50% of that of wild-type P protein (Dupuy et al. (1999) J. Virol. 73:8384-8392). Replacement of both residues at positions 232 and 237 with alanine has no significant impact on RNA transcription and replication.

The present invention provides RSV viruses and P protein in which the serine residues in the P protein were altered to eliminate their phosphorylation potential. Exemplary embodiments include recombinant RSVs with mutations of serines at two (232/237): rA2-PP2; or five (116/117/119/232/237):rA2-PP5, of the P protein phosphorylation sites. For example, serines at positions 116, 117, 119, and 232, 237, were changed to LRL, and AA, respectively. Alternatively, these two clusters of serines were changed to aspartic acid to mimic the negative charges. Similar activity levels are observed for P protein with S232D/S237D or S232A/S237A substitutions. In contrast, substitutions of the three serines at 116, 117, and 119 by aspartic acid completely abolished P protein function, with a single S116D change having the most significant effect. Substitutions of the same residues by LRL had only a slight effect on P protein function.

Variants of the RSV A2 strain with amino acid substitutions eliminating either two phosphorylation sites (S232A; S237A [PP2]) or five phosphorylation sites (S116L; S117R; S119L; S232A; S237A [PP5]) exhibit reduced phosphorylation. Immunoprecipitation of ³³P-labeled infected cells showed that P protein phosphorylation was reduced by 80% for rA2-PP2 and 95% for rA2-PP5. Although the two recombinant viruses replicated well in Vero cells, rA2-PP2 and, to a greater extent, rA2-PP5, replicated poorly in HEp-2 cells. Virus budding from the infected HEp-2 cells was affected by dephosphorylation of P protein, because the majority of rA2-PP5 remained cell associated. In addition, rA2-PP5 was also more attenuated than rA2-PP2 in replication in the respiratory tracts of mice and cotton rats.

Coimmunoprecipitation analysis indicated that interactions of the N and P proteins were reduced by dephosphorylation of P protein. A reduction of about 30% is observed in the N-P interaction of rA2-PP2, from which the two major phosphorylation sites had been removed, and a reduction of about 60% is observed in the N-P protein interaction for rA2-PP5, from which all five phosphorylation sites had been removed. This observation is consistent with a previous report in which alteration of S-232 and S-237 reduced the ability of P protein to interact with N protein by about 50% in a two-hybrid system (Slack & Easton (1998) Virus Research 55:167-176).

Viral RNA transcription and replication are also affected by P protein phosphorylation as evidenced by an increase in rA2-PP5 mRNA in infected cells, along with a concomitant reduction in genomic RNA synthesis. The reduced RNA synthesis in rA2-PP5 infected HEp-2 cells is likely to be due a reduction in the efficiency of replication. The minigenome analysis suggested that a slightly lower antigenome/mRNA ratio correlated with the LRL change.

Infections virus rA2-PP5 replicates efficiently in Vero cells, making it unlikely that RSV P protein oligomerization was affected by P protein phosphorylation. However, removal of the major phosphorylation sites from P protein significantly reduces virus budding from rA2-PP5-infected cells, with the majority of viruses remaining cell associated, rA2-PP5 is unable to sustain extensive in vitro passaging following infection of susceptible cells, and is highly attenuated in mice and cotton rats, consistent with suitability for attenuated vaccine formulations.

### Recombinant RSV with Mutations in P, Nucleic Acids and Vaccines

One aspect of the present invention provides recombinant respiratory syncytial viruses that exhibit an attenuated phenotype and that comprise a mutated phosphoprotein. Another aspect of the present invention provides live attenuated RSV vaccines comprising such recombinant RSV. Recombinant phosphoproteins and nucleic acids encoding such recombinant phosphoproteins and/or recombinant viruses are also features of the invention.

Thus, one general class of embodiments provides a recombinant respiratory syncytial virus having an attenuated phenotype and comprising a phosphoprotein (P) that comprises at least one artificially mutated amino acid residue. For example, the phosphoprotein can comprise a deletion of at least one amino acid residue, an insertion of at least one amino acid residue, and/or at least one substituted amino acid residue (e.g., an amino acid residue occupying a particular position in a wild-type protein can be replaced by another of the twenty naturally occurring amino acids or by a nonnatural amino acid).

In one class of embodiments, the phosphoprotein comprises at least one mutated amino acid residue at a position selected from the group consisting of position 172, position 174, position 175 and position 176. For example, the phosphoprotein can comprise at least one substituted amino acid residue at a position selected from the group consisting of position 172, position 174, position 175 and position 176. The phosphoprotein can comprise, e.g., a glycine to serine substitution at position 172 (G172S). The phosphoprotein can comprise, e.g., an arginine to alanine substitution at position 174 (R174A). The phosphoprotein can comprise, e.g., a glutamic acid to alanine substitution at position 175 (E175A). The phosphoprotein can comprise, e.g., a glutamic acid to glycine substitution at position 176 (E176G), a glutamic acid to alanine substitution at position 176 (E176A), a glutamic acid to aspartic acid substitution at position 176 (E176D), a glutamic acid to cysteine substitution at position 176 (E176C) or a glutamic acid to serine substitution at position 176 (E176S). The phosphoprotein can comprise substituted amino acid residues at two or more of these positions; for example, the phosphoprotein can comprise substituted amino acid residues at positions 172 and 176.

In a related class of embodiments, the phosphoprotein comprises a plurality of substituted ammo acid residues, which residues are selected from residues 172-176. For example, the phosphoprotein can comprise an arginine to alanine substitution at position 174 (R174A), a glutamic acid to alanine substitution at position 175 (E175A), and a glutamic acid to alanine substitution at position 176 (E176A).

In one class of embodiments, the phosphoprotein comprises a deletion of a plurality of amino acid residues selected from residues 172-176. For example, the phosphoprotein can comprise a deletion of amino acid residues 172-176. As another example, the phosphoprotein can comprise a deletion of amino acid residues 161-180.

In a similar class of embodiments, the phosphoprotein comprises a deletion of a plurality of amino acid residues selected from residues 236-241. For example, the phosphoprotein can comprise a deletion of amino acid residues 236-241.

In one class of embodiments, the attenuated recombinant RSV comprises a phosphoprotein comprising at least one mutated amino acid residue that eliminates a phosphorylation site. For example, the phosphoprotein can comprise at least one substituted amino acid residue that eliminates a phosphorylation site. In a preferred class of embodiments, the at least one substituted amino acid residue replaces a serine; for example, the at least one substituted amino acid residue can replace a serine at one or more positions selected from the group consisting of positions 116, 117, 119, 232 and 237. The phosphoprotein can comprise, e.g., amino acid substitution S116D, amino acid substitution S116A or amino acid substitution S116L. The phosphoprotein can comprise, e.g., amino acid substitution S117D, amino acid substitution S117A, or amino acid substitution S117R. The phosphoprotein can comprise, e.g., amino acid substitution S119D, amino acid substitution S119A, or amino acid substitution S119L. The phosphoprotein can comprise, e.g., amino acid substitution S232A or amino acid substitution S232D. The phosphoprotein can comprise, e.g., amino acid substitution S237A or amino acid substitution S237D.

In some embodiments, the phosphoprotein comprises two or more substituted amino acid residues. For example, substituted amino acid residues can replace serines at positions 117 and 119; for example, the phosphoprotein can comprise an amino acid substitution selected from the group consisting of S117A, S117D and S117R and an amino acid substitution selected from the group consisting of S119A, S119D and S119L (e.g., the phosphoprotein can comprise amino acid substitutions S117A and S119A).

As another example, substituted amino acid residues can replace serines at positions 116, 117 and 119. The substituted amino acid residue at position 116 can, e.g., be selected from the group consisting of alanine (S116A), aspartic acid (S116D) and leucine (S116L). The substituted amino acid residue at position 117 can, e.g., be selected from the group consisting of alanine (S117A), aspartic acid (S117D) and arginine (S117R). The substituted amino acid residue at position 119 can, e.g., be selected from the group consisting of alanine (S119A), aspartic acid (S119D) and leucine (S119L). For example, the phosphoprotein can comprise an amino acid substitution selected from the group consisting of S116L, S116A, and S116D; an amino acid substitution selected from the group consisting of S117R, S117A, and S117D; and an amino acid substitution selected from the group consisting of S119L, S119A, and S119D (e.g., the phosphoprotein can comprise amino acid substitutions S116D, S117D and S119D or amino acid substitutions S116L, S117R and S119L).

As yet another example, substituted amino acid residues can replace serines at positions 232 and 237. The substituted amino acid residue at position 232 can, e.g., be selected from the group consisting of alanine (S232A) and aspartic acid (S232D). The substituted amino acid residue at position 237 can, e.g., be selected from the group consisting of alanine (S237A) and aspartic acid (S237D). For example, the phosphoprotein can comprise an amino acid substitution selected from the group consisting of S232A and S232D and an amino acid substitution selected from the group consisting of S237A and S237D (e.g., the phosphoprotein can comprise amino acid substitutions S232D and S237D or amino acid substitutions S232A and S237A).

As yet another example, substituted amino acid residues can replace serines at positions 116, 117, 119, 232 and 237. The substituted amino acid residue at position 116 can, e.g., be selected from the group consisting of leucine (S116L), alanine (S116A) and aspartic acid (S116D). The substituted amino acid residue at position 117 can, e.g., be selected from the group consisting of arginine (S117R), alanine (S117A) and aspartic acid (S117D). The substituted amino acid residue at position 119 can, e.g., be selected from the group consisting of leucine (S119L), alanine (S119A) and aspartic acid (S119D). The substituted amino acid residue at position 232 can, e.g., be selected from the group consisting of alanine (S232A) and aspartic acid (S232D). The substituted amino acid residue at position 237 can, e.g., be selected from the group consisting of alanine (S237A) and aspartic acid (S237D). For example, the phosphoprotein can comprise an amino acid substitution selected from the group consisting of S116L, S116A, and S116D; an amino acid substitution selected from the group consisting of S117R, S117A, and S117D; an amino acid substitution selected from the group consisting of S119L, S119A, and S119D; an amino acid substitution selected from the group consisting of S232A and S232D; and an amino acid substitution selected from the group consisting of S237A and S237D (e.g., the phosphoprotein can comprise amino acid substitutions S116L, S117R, S119L, S232A and S237A or amino acid substitutions S116L, S117R, S119L, S232D and S237D).

The recombinant RSV can comprise any species, subgroup and/or strain of RSV. In preferred embodiments, the recombinant RSV comprises a human RSV of subgroup A, subgroup B or a chimera thereof.

Nucleic acids provide another feature of the invention. One class of embodiments provides a nucleic acid encoding a recombinant respiratory syncytial virus having an attenuated phenotype and comprising a phosphoprotein that comprises at least one mutated amino acid residue. The nucleic acid can be, e.g., a DNA (e.g., a cDNA) or an RNA. The nucleic acid can be an RSV genome or antigenome. A vector (e.g., a plasmid) can comprise the nucleic acid.

Another aspect of the invention provides artificially mutated phosphoproteins (e.g., those described above). Yet another aspect provides nucleic acids encoding the artificially mutated phosphoproteins. The variations noted above apply to these nucleic acids as well; thus, the nucleic acid can be a DNA (e.g., a cDNA) or an RNA, can be an RSV genome or antigenome and/or can comprise a vector (e.g., a plasmid).

The present invention also provides vaccines comprising attenuated recombinant RSV. One class of embodiments provides a live attenuated respiratory syncytial virus vaccine comprising an immunologically effective amount of a recombinant respiratory syncytial virus having an attenuated phenotype and comprising a phosphoprotein (P) that comprises at least one mutated amino acid residue. The vaccine optionally further comprises a physiologically acceptable carrier and/or an adjuvant.

In other embodiments, the invention provides methods for stimulating the immune system of an individual to produce an immune response against RSV. The methods comprise administering to the individual a recombinant respiratory syncytial virus, the virus having an attenuated phenotype and comprising a phosphoprotein (P) that comprises at least one mutated amino acid residue, in a physiologically acceptable carrier. In preferred embodiments, the immune response is a protective immune response. The vaccine can be administered in one or more doses to achieve the desired level of protection. The recombinant RSV is preferably administered to the upper respiratory tract (e.g., the nasopharynx) of the individual, and is preferably administered by spray, droplet or aerosol.

### Methods that Can Produce Attenuated Recombinant RSV

One aspect of the present invention provides methods of identifying a phosphoprotein or nucleoprotein having altered interaction with another protein. In the methods, a plurality of protein variants are provided, in which each protein variant comprises at least a portion of a first RSV protein. The first RSV protein is selected from the group consisting of an RSV phosphoprotein and an RSV nucleoprotein. At least one candidate protein variant is identified that has an altered interaction with a second RSV protein or portion thereof. The portion of the first RSV protein typically comprises one or more domains, but can comprise anywhere from a few amino acid residues up to the entire full-length protein. The variants can further comprise additional useful polypeptide sequences, for example, one or more tags (e.g., a poly-histidine tag, an epitope tag), a GST moiety, and/or a DNA-binding or activation domain. The variants can each comprise the same or different size portions of the first protein.

In one class of embodiments, a plurality of protein variants are provided, in which each protein variant comprises at least a portion of a first RSV protein. The portion of the first RSV protein comprises at least one artificial mutation. (e.g., at least one mutated amino acid residue, e.g., one or more substituted, inserted or deleted amino acid residues). The first RSV protein is selected from the group consisting of an RSV phosphoprotein and an RSV nucleoprotein. At least one candidate protein variant is identified that has an altered interaction with a second RSV protein or portion thereof. In certain embodiments, the first RSV protein is an RSV phosphoprotein and the second RSV protein is an RSV nucleoprotein. In other similar embodiments, the first RSV protein is an RSV nucleoprotein and the second RSV protein is an RSV phosphoprotein.

The at least one candidate protein variant having an altered interaction with a second RSV protein can be identified by performing an in vivo assay (e.g., a two hybrid assay). Alternatively, the at least one candidate protein variant having an altered interaction with a second RSV protein can be identified by performing an in vitro assay (e.g., coimmunoprecipitation, GST pulldown, far Western, or the like). The candidate protein variant having an altered interaction with the second RSV protein can have an increased or, preferably, decreased interaction with the second protein. The decrease can be quantitative (e.g., a 10-fold or 100-fold decrease in binding affinity as measured in an in vitro assay) or qualitative (e.g., failure to grow a two hybrid assay). In certain embodiments, the interaction is altered in a temperature-dependent manner (e.g., the mutant can be ts or cs).

The methods can comprise additional steps. For example, the nature of the at least one mutation in the portion of the first RSV protein comprising at least one of the candidate protein variants can be determined. The methods can lead to the production of recombinant RSV, including attenuated recombinant RSV. Thus, at least one recombinant RSV can be produced. The genome or antigenome of the recombinant virus encodes a phosphoprotein or a nucleoprotein that comprises the at least one mutation in the portion of the first RSV protein comprising at least one of the candidate protein variants. One of skill will recognize that the candidate protein variant can, in some instances, comprise two or more mutations, only one of which need be introduced into the recombinant RSV if desired. The mutation(s) in the candidate variant and in the recombinant RSV need not be the same on the nucleic acid level, as long as the encoded proteins comprise the desired mutation(s).

Replication of the recombinant RSV can be assessed to identify at least one recombinant RSV having a reduced level of replication, e.g., a recombinant RSV whose replication is reduced at least 10-fold or even at least 100-fold, e.g., as compared to a wild-type, naturally circulating strain of RSV and/or to the RSV strain into which the mutation was introduced. Replication can be assessed, for example, by determining peak titer of the virus. Replication can be assessed in cultured cells, in an animal (e.g., in the upper and/or lower respiratory tract), and/or in a human (e.g., in the upper and/or lower respiratory tract). Suitable animal models include a rodent (e.g., a mouse, a cotton rat) or a primate (e.g., an African green monkey, a chimpanzee). Methods for determining levels of RSV (e.g., in the nasopharynx and/or in the lungs) of an infected host (e.g., human or animal) are well known in the literature. Specimens are obtained, for example, by aspiration or washing out of nasopharyngeal secretions, and virus is quantified in tissue culture or other by laboratory procedure. See, for example, Belshe et al., J. Med. Virology 1:157-162 (1977), Friedewald et al., J. Amer. Med. Assoc. 204:690-694 (1968); Gharpure et al., J. Virol. 3:414-421 (1969); and Wright et al., Arch. Ges. Virusforsch. 41:238-247 (1973).

### Functional Mutations in the M2-1 Protein

Unlike other members in Paramyxoviridae family, efficient transcription of RSV mRNA requires an additional protein, M2-1, (Collins et al. (1996) Proc, Natl. Acad. Sci. USA 93:81-85). M2-1 is encoded by the first of the two overlapping open reading frames of M2 mRNA (Ahmadian et al. (2000) EMBO J. 19:2681-2689; Collins & Wertz (1985) Virology 54:65-71). The M2-1 protein of respiratory syncytial virus (RSV) is a transcription antiterminator that is essential for virus replication. It functions as transcriptional processivity factor to prevent premature termination during transcription (Collins et al. (1996) Proc Natl Acad Sci. USA 93:81-85; Fearns & Collins (1999) J. Virol. 73:388-397; Fearns & Collins (1999) J. Virol. 73:5833-5864) and enhances transcriptional read-through at gene junctions (Hardy et al. (1999) J. Virol. 73:170-176; Hardy & Wertz (2000) J. Virol. 74:5880-5885; Hardy & Wertz (1998) J. Virol. 72:520-526), which permits access of the RSV polymerase to the downstream transcriptional units. Functional M2-1 is essential for RSV replication; certain alterations of its sequence destroy virus infectivity (Tang et al. (2001) J. Virol. 75:11328-11335).

The M2-1 protein of hRSV A2 strain is 194 amino acids in length with a molecular weight of approx. 22,150 (Collins et al. (1990) J. Gen. Virol. 71:3015-3020; Collins & Wertz (1985) J. Virol. 54:65-71). It contains a Cys₃-His₁ motif in the N-terminus, that is highly conserved among human, bovine, ovine and murine strains of pneumoviruses (Ahmadian et al. 2000, EMBO J. 19:2681-2689; Alansari & Potgieter. 1994, J. Gen. Virol. 75:3597-3601; van den Hoogen et al. 2002, Virology 295:119-132; and Yu et al. 1995, J Virol 69:2412-2419). The M2-1 function requires its interaction with the N and P proteins. Recent studies have demonstrated a direct interaction between the M2-1 and N proteins that is mediated through RNA (Cartee & Wertz. 2001, J. Virol 75:12188-12197; and Cuesta et al. 2000, J Virol 74:9858-9867). Substitutions of the three cysteines and one histidine in this motif significantly reduced the ability of M2-1 to enhance transcription read-through and disrupted the interaction between the M2-1 and N proteins (Hardy & Wertz (2000) J. Virol. 74;170-176), which is lethal to virus replication (Tang et al. (2001) J. Virol. 75:11328-11335). However, despite conservation of the Cys3-His1 motif, there is a striking difference in the processivity of transcription between species of pneumovirus, indicating that the Cys3-His1 motif alone is not sufficient for M2-1 function. Construction of chimeras incorporating sequence elements of the M2-1 proteins of RSV and pneumovirus of mouse (PVM) demonstrated that additional residues at the N-terminus play an important role in determining protein function. For example, chimeras including the N-terminal 30 amino acids of RSV with the remaining 148 amino acids of PVM M2-1 (RP M2-1) maintained a good level of activity, whereas chimeras including the 29 N-terminal amino acids of PVM with the C-terminal 164 amino acids from RSV (PR M2-1) had little activity regardless of conservation of the Cys₃His₁ motif.

The present invention provides RSV M2-1 mutants (isolated proteins and recombinant virus) with amino acid substitutions in the N-terminal residues which are essential for the RSV M2-1 function. For example, RSV M2-1 proteins comprising amino acid substitutions of serine for leucine at position 16 (L16S) and/or of arginine for asparagine at position 17 (N17R) have significantly reduced M2-1 function. For example, substitution of serine for leucine at position 16 results in a 97% reduction in protein function, while a substitution of arginine for asparagine at position 17 results in a 94% reduction in protein function. RSV M2-1 protein comprising both the L16S and N17R mutations exhibits only 1% residual activity. Such reductions in M2-1 function correspond with an attenuated viral phenotype desirable in the production of live attenuated vaccines.

One aspect of the present invention provides recombinant respiratory syncytial viruses that exhibit an attenuated phenotype and that comprise an artificially mutated M2-1 protein. Another aspect of the present invention provides live attenuated RSV vaccines comprising such recombinant RSV. Recombinant M2-1 proteins and nucleic acids encoding such recombinant M2-1 proteins and/or recombinant viruses are also features of the invention.

Thus, one general class of embodiments provides a recombinant respiratory syncytial virus having an attenuated phenotype and comprising an M2-1 protein that comprises at least one artificially mutated amino acid residue at a position (i.e., an amino acid residue position) selected from the group consisting of position 3, position 12, position 14, position 16, position 17, and position 20. For example, the mutated residue(s) can be deleted or substituted (e.g., an amino acid residue occupying a particular position in a wild-type protein can be replaced by another of the twenty naturally occurring amino acids or by a nonnatural amino acid). Thus, in one class of embodiments, the M2-1 protein comprises at least one substituted amino acid residue at a position selected from the group consisting of position 3, position 12, position 14, position 16, position 17, and position 20. The M2-1 protein can comprise, e.g., an arginine to valine substitution at position 3 (R3V), an arginine to glutamine substitution at position 12 (R12Q), a histidine to phenylalanine substitution at position 14 (H14F), a leucine to serine substitution at position 16 (L16S), an asparagine to arginine substitution at position 17 (N17R) and/or an arginine to asparagine substitution at position 20 (R20N).

The M2-1 protein can comprise substituted amino acid residues at two or more of these positions, as indicated by the following examples. The M2-1 protein can comprise amino acid substitutions L16S and N17R. The M2-1 protein can comprise amino acid substitutions R12Q and H14F. The M2-1 protein can comprise amino acid substitutions R12Q and R20N. The M2-1 protein can comprise amino acid substitutions H14F and R20N. The M2-1 protein can comprise amino acid substitutions R12Q, H14F and R20N.

The recombinant RSV can comprise any species, subgroup and/or strain of RSV. In preferred embodiments, the recombinant RSV comprises a human RSV of subgroup A, subgroup B or a chimera thereof.

Nucleic acids provide another feature of the invention. One class of embodiments provides a nucleic acid encoding a recombinant respiratory syncytial virus having an attenuated phenotype and comprising an M2-1 protein that comprises at least one mutated amino acid residue at a position selected from the group consisting of position 3, position 12, position 14, position 16, position 17, and position 20. The nucleic acid can be, e.g., a DNA (e.g., a cDNA) or an RNA. The nucleic acid can be an RSV genome or antigenome. A vector (e.g., a plasmid) can comprise the nucleic acid.

Artificially mutated M2-1 proteins (e.g., those described above) provide another feature of the invention. Nucleic acids encoding the artificially mutated M2-1 proteins provide yet another feature of the invention. The variations noted above apply to these nucleic acids as well; thus, the nucleic acid can be a DNA (e.g., a cDNA) or an RNA, can be an RSV genome or antigenome and/or can comprise a vector (e.g., a plasmid).

The present invention also provides vaccines comprising attenuated recombinant RSV. One class of embodiments provides a live attenuated respiratory syncytial virus vaccine comprising an immunologically effective amount of a recombinant respiratory syncytial virus having an attenuated phenotype and comprising an M2-1 protein that comprises at least one mutated amino acid residue at a position selected from the group consisting of position 3, position 12, position 14, position 16, position 17, and position 20. The vaccine optionally further comprises a physiologically acceptable carrier and/or an adjuvant.

In other embodiments, the invention provides methods for stimulating the immune system of an individual to produce an immune response against RSV. The methods comprise administering to the individual a recombinant respiratory syncytial virus, the virus having an attenuated phenotype and comprising an M2-1 protein that comprises at least one mutated amino acid residue at a position selected from the group consisting of position 3, position 12, position 14, position 16, position 17, and position 20, in a physiologically acceptable carrier. In preferred embodiments, the immune response is a protective immune response. The vaccine can be administered in one or more doses to achieve the desired level of protection. The recombinant RSV is preferably administered to the upper respiratory tract (e.g., the nasopharynx) of the individual, and is preferably administered by spray, droplet or aerosol.

Another general class of embodiments provides a recombinant RSV having an attenuated phenotype and comprising a chimeric M2-1 protein, which chimeric M2-1 protein comprises a plurality of residues from an RSV M2-1 protein and a plurality of residues from a pneumonia virus of mice (PVM) M2-1 protein. In one class of embodiments, the chimeric M2-1 protein comprises a plurality of residues from the N-terminal region (i.e., a plurality of residues from the N-terminal half) of the RSV M2-1 protein and a plurality of residues from the C-terminal region (i.e., a plurality of residues from the C-terminal half) of the PVM M2-1 protein. For example, in one specific embodiment, the chimeric M2-1 protein comprises the N-terminal 30 residues of the RSV M2-1 protein and the C-terminal 148 residues of the PVM M2-1 protein. In another class of embodiments, the chimeric M2-1 protein comprises a plurality of residues from the N-terminal region (half) of the PVM M2-1 protein and a plurality of residues from the C-terminal region (half) of the RSV M2-1 protein. In one embodiment, the chimeric M2-1 protein comprises the N-terminal 29 residues of the PVM M2-1 protein and the C-terminal 164 residues of the RSV M2-1 protein.

The chimeric proteins can further comprise one or more amino acid substitutions, insertions, and/or deletions. For example, the chimeric M2-1 protein comprising the N-terminal 29 residues of the PVM M2-1 protein and the C-terminal 164 residues of the RSV M2-1 protein can further comprise at least one substituted amino acid residue at a position selected from the group consisting of position 3, position 11, position 13, position 15, position 16, position 19 and position 25, as illustrated by the following examples. The chimeric M2-1 protein can comprise a valine to arginine substitution at position 3 (V3R). The chimeric M2-1 protein can comprise a glutamine to arginine substitution at position 11 (Q11R). The chimeric M2-1 protein can comprise a serine to leucine substitution at position 15 (S15L). The chimeric M2-1 protein can comprise an arginine to asparagine substitution at position 16 (R16N). The chimeric M2-1 protein can comprise an asparagine to arginine substitution at position 19 (N19R). The chimeric M2-1 protein can comprise amino acid substitutions S15L and R16N. The chimeric M2-1 protein can comprise amino acid substitutions Q11R and F13H. The chimeric M2-1 protein can comprise amino acid substitutions Q11R, F13H, and N19R. The chimeric M2-1 protein can comprise amino acid substitutions V3R, S15L and R16N. The chimeric M2-1 protein can comprise amino acid substitutions Q11R, S15L and R16N. The chimeric M2-1 protein can comprise amino acid substitutions S15L, R16N and N19R. The chimeric M2-1 protein can comprise amino acid substitutions Q11R, F13H, S15L and R16N. The chimeric M2-1 protein can comprise amino acid substitutions Q11R, F13H, S15L, R16N and N19R.

The recombinant RSV can comprise any species, subgroup and/or strain of RSV. In preferred embodiments, the recombinant RSV comprises a human RSV of subgroup A, subgroup B or a chimera thereof.

Nucleic acids provide another feature of the invention. One class of embodiments provides a nucleic acid encoding a recombinant respiratory syncytial virus having an attenuated phenotype and comprising a chimeric M2-1 protein that comprises a plurality of residues from an RSV M2-1 protein and a plurality of residues from a pneumonia virus of mice (PVM) M2-1 protein. The nucleic acid can be, e.g., a DNA (e.g., a cDNA) or an RNA. The nucleic acid can be an RSV genome or antigenome. A vector (e.g., a plasmid) can comprise the nucleic acid.

The chimeric M2-1 proteins described above provide another feature of the invention. Nucleic acids encoding the chimeric M2-1 proteins provide yet another feature of the invention. The variations noted above apply to these nucleic acids as well; thus, the nucleic acid can be a DNA (e.g., a cDNA) or an RNA, can be an RSV genome or antigenome and/or can comprise a vector (e.g., a plasmid).

The present invention also provides vaccines comprising attenuated recombinant RSV. One class of embodiments provides a live attenuated respiratory syncytial virus vaccine comprising an immunologically effective amount of a recombinant respiratory syncytial virus having an attenuated phenotype and comprising a chimeric M2-1 protein that comprises a plurality of residues from an RSV M2-1 protein and a plurality of residues from a pneumonia virus of mice (PVM) M2-1 protein. The vaccine optionally further comprises a physiologically acceptable carrier and/or an adjuvant.

In other embodiments, the invention provides methods for stimulating the immune system of an individual to produce an immune response against RSV. The methods comprise administering to the individual a recombinant respiratory syncytial virus, the virus having an attenuated phenotype and comprising a chimeric M2-1 protein that comprises a plurality of residues from an RSV M2-1 protein and a plurality of residues from a pneumonia virus of mice (PVM) M2-1 protein, in a physiologically acceptable carrier. In preferred embodiments, the immune response is a protective immune response. The vaccine can be administered in one or more doses to achieve the desired level of protection. The recombinant RSV is preferably administered to the upper respiratory tract (e.g., the nasopharynx) of the individual, and is preferably administered by spray, droplet or aerosol.

One aspect of the invention provides methods of identifying an M2-1 protein having an altered activity. In the methods, one or more chimeric M2-1 proteins are provided. Each chimeric M2-1 protein comprises a plurality of residues from an RSV M2-1 protein from a first strain of virus and a plurality of residues from an M2-1 protein from a second strain of virus. At least one candidate chimeric M2-1 protein having an altered activity is identified.

The first and second strains of virus can be different strains of RSV (e.g., one strain of subgroup A and one strain of subgroup B). Alternatively, the first and second strains of virus can be different species of virus (e.g., the first strain is an RSV, and the second strain can be a pneumovirus or a metapneumovirus). For example, at least one of the chimeric M2-1 proteins can comprise a plurality of residues from, an RSV M2-1 protein and a plurality of residues from a pneumonia, virus of mice (PVM) M2-1 protein. The chimeric M2-1 protein can comprise a plurality of residues from the N-terminal region (half) of the RSV M2-1 protein and a plurality of residues from the C-terminal region (half) of the PVM M2-1 protein. Alternatively, the chimeric M2-1 protein can comprise a plurality of residues from the N-terminal region (half) of the PVM M2-1 protein and a plurality of residues from the C-terminal region (half) of the RSV M2-1 protein.

The at least one candidate chimeric M2-1 protein having an altered activity can be identified, for example, by assaying M2-1-dependent processivity (e.g., in a minigenome assay), by assaying RNA binding by the candidate chimeric M2-1 protein (e.g., in a gel shift assay), and/or by assaying nucleoprotein binding by the candidate chimeric M2-1 protein (e.g., by coimmunoprecipitation). The activity of the M2-1 protein can be increased, or, typically, decreased.

The method can lead to the production of recombinant RSV, including attenuated recombinant RSV. Thus, at least one recombinant respiratory syncytial virus (RSV) whose genome or antigenome encodes at least one candidate chimeric M2-1 protein can be produced. Replication of the recombinant RSV can be assessed to identify at least one recombinant RSV having a reduced level of replication, e.g., a recombinant RSV whose replication is reduced at least 10-fold or even at least 100-fold, e.g., as compared to a wild-type, naturally circulating strain of RSV and/or to the RSV strain into which the chimeric M2-1 protein was introduced. Replication can be assessed, for example, by determining peak titer of the virus. Replication can be assessed in cultured cells, in an animal (e.g., in the upper and/or lower respiratory tract), and/or in a human (e.g., in the upper and/or lower respiratory tract). Suitable animal models include a rodent (e.g., a mouse, a cotton rat) or a primate (e.g., an African green monkey, a chimpanzee). Methods for determining levels of RSV (e.g., in the nasopharynx and/or in the lungs) of an infected host (e.g., human or animal) are well known in the literature. Specimens are obtained, for example, by aspiration or washing out of nasopharyngeal secretions, and virus is quantified in tissue culture or other by laboratory procedure. See, for example, Belshe et al., J. Med. Virology 1:157-162 (1977), Friedewald et al., J. Amer. Med. Assoc. 204:690-694 (1968); Gharpure et al., J. Virol. 3:414-421 (1969); and Wright et al., Arch. Ges. Virusforsch. 41:238-247 (1973).

One or more mutations can be introduced into at least one of the candidate chimeric M2-1 proteins, and at least one mutated candidate chimeric M2-1 protein can be identified wherein the altered activity is further altered (typically, a decreased activity exhibited by the candidate chimeric M2-1 protein is further decreased for the mutated candidate chimeric M2-1 protein). At least one recombinant respiratory syncytial virus whose genome or antigenome encodes at least one mutated candidate chimeric M2-1 protein can be produced, and its replication assessed as described.

If desired, mutations affecting the activity of the mutated candidate chimeric M2-1 protein can be introduced into an RSV M2-1 (e.g., a non-chimeric M2-1). Thus, the methods can further comprise introducing one or more mutations into at least one RSV M2-1 protein, and identifying at least one candidate mutated RSV M2-1 protein having an altered activity. At least one recombinant respiratory syncytial virus whose the genome or antigenome encodes at least one candidate mutated RSV M2-1 protein can be produced, and its replication assessed as described.

Any of the mutations (e.g., amino acid substitutions or deletions) in the RSV M2-1 and P proteins described herein can optionally be combined with any other mutation(s) in an RSV (e.g., mutations altering noncoding sequences, mutations such as amino acid substitutions, insertions or deletions in viral proteins, etc.) to result, e.g., in an attenuated RSV possessing the desired degree of attenuation while retaining the ability to induce a protective immune response.

When referring herein to specific positions of the RSV phosphoprotein (P) and M2-1 and M2-2 proteins, positions are numbered as in the P, M2-1 and M2-2 proteins of RSV strain A2. The P, M2-1 and/or M2-2 proteins of other species, strains and/or subgroups may contain, e.g., one or more amino acid deletions and/or insertions such that they do not have the same number of residues as the strain A2 proteins. In such a case, the relevant position of the other virus's P, M2-1 or M2-2 can be determined by alignment with the RSV A2 P, M2-1 or M2-2. Alignment can be performed by means well known in the art, e.g., visual inspection (see generally, Ausubel et al., infra) or a sequence comparison algorithm (e.g., the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Natl. Acad. Sci. USA 85:2444 (1988), the BLAST algorithm described in Altschul et al., J. Mol. Biol. 215:403-410 (1990), or by computerized implementations of these algorithms, such as GAP, BESTFIT, FASTA, and TFASTA In the Wisconsin Genetics Software Package, Genetics Computer Group, 575 Science Dr., Madison, Wis., or BLAST software publicly available through the National Center for Biotechnology Information (www.ncbi.nlm.nih.gov)).

### Functional Mutation in the M2-2 Protein

The M2-2 protein has been implicated in regulating RSV RNA replication and transcription in the virus life cycle (Jin et al. (2000) J. Virol 74:74-82 and Bermingham and Collins (1999) Proc Natl Acad Sci USA 96:11259-11264). Deletion of the M2-2 ORF from RSV affects virus replication in HEp-2 cells, but not in Vero cells (Jin et al. (2000) J Virol 74:74-82). The M2-2-deleted RSV is also attenuated in animals, suggesting that RSV M2-2 deletion virus is a vaccine candidate (Jin et al. (2000) J Virol 74:74-82; Cheng et al. (2001) Virology 283:59-68; and Jin et al. (2003) Vaccine 121:3647-3652). The M2-2 protein is encoded by the M2 gene; its open reading frame overlaps with the upstream M2-1 ORF.

The present invention provides RSV M2-2 mutants (isolated proteins and recombinant viruses) with amino acid deletions, insertions and/or substitutions that reduce M2-2 function (e.g., in a minigenome assay as described in Example 3 below). Such reductions in M2-2 function can correspond to an attenuated viral phenotype desirable in the production of live attenuated vaccines.

One aspect of the present invention provides recombinant respiratory syncytial viruses that exhibit an attenuated phenotype and that comprise a mutated M2-2 protein. Another aspect of the present invention provides live attenuated RSV vaccines comprising such recombinant RSV. Recombinant M2-2 proteins and nucleic acids encoding such recombinant M2-2 proteins and/or recombinant viruses are also features of the invention.

Thus, one general class of embodiments provides a recombinant respiratory syncytial virus having an attenuated phenotype and comprising an M2-2 protein that comprises at least one artificially mutated amino acid residue. For example, the M2-2 protein can comprise a deletion of at least one amino acid residue, an insertion of at least one amino acid residue, and/or at least one substituted amino acid residue.

In one class of embodiments, the M2-2 protein comprises at least one mutated amino acid residue at a position selected from the group consisting of position 1, position 3 and position 7. In one class of embodiments, the M2-2 protein comprises a deletion of amino acid residues 1-2 (e.g., when the first and optionally third AUG in the M2-2 mRNA is mutated such that translation is forced to begin at the second AUG). In another class of embodiments, the M2-2 protein comprises a deletion of amino acid residues 1-6 (e.g., when the first and second AUGs in the M2-2 mRNA are mutated such that translation is forced to begin at the third AUG).

In a similar class of embodiments, the M2-2 protein comprises a deletion selected from the group consisting of a deletion of the N-terminal 6 amino acid residues, a deletion of the N-terminal 8 amino acid residues, a deletion of the N-terminal 10 amino acid residues, a deletion of the C-terminal 1 amino acid residue, a deletion of the C-terminal 2 amino acid residues, a deletion of the C-terminal 4 amino acid residues, a deletion of the C-terminal 8 amino acid residues, and a deletion of the C-terminal 18 amino acid residues. The M2-2 protein can optionally comprise a combination of such N- and C-terminal deletions.

In one class of embodiments, the M2-2 protein comprises at least one artificially mutated amino acid residue at position 2, position 4, position 5, position 6, position 11, position 12, position 15, position 25,position 27, position 34, position 47, position 56, position 58, position 66, position 75, position 80 and/or position 81. For example, the M2-2 protein can comprise at least one amino acid substitution selected from the group consisting of T2A, P4A, K5A, I6A, I6K, D11A, K12A, C15A, R25A, R27A, K34A, H47A, E56A, H58A, D66A, H75A, E80A and D81A.

The recombinant RSV can comprise any species, subgroup and/or strain of RSV. In preferred embodiments, the recombinant RSV comprises a human RSV of subgroup A, subgroup B or a chimera thereof.

Nucleic acids provide another feature of the invention. One class of embodiments provides a nucleic acid encoding a recombinant respiratory syncytial virus having an attenuated phenotype and comprising an M2-2 protein that comprises at least one mutated amino acid residue. The nucleic acid can be, e.g., a DNA (e.g., a cDNA) or an RNA. The nucleic acid can be an RSV genome or antigenome. A vector (e.g., a plasmid) can comprise the nucleic acid.

Another aspect of the invention provides artificially mutated M2-2 proteins (e.g., those described above). Yet another aspect provides nucleic acids encoding the artificially mutated M2-2 proteins. The variations noted above apply to these nucleic acids as well; thus, the nucleic acid can be a DNA (e.g., a cDNA) or an RNA, can be an RSV genome or antigenome and/or can comprise a vector (e.g., a plasmid).

The present invention also provides vaccines comprising attenuated recombinant RSV. One class of embodiments provides a live attenuated respiratory syncytial virus vaccine comprising an immunologically effective amount of a recombinant respiratory syncytial virus having an attenuated phenotype and comprising an M2-2 protein that comprises at least one mutated amino acid residue. The vaccine optionally further comprises a physiologically acceptable carrier and/of an adjuvant.

In other embodiments, the invention provides methods for stimulating the immune system of an individual to produce an immune response against RSV. The methods comprise administering to the individual a recombinant respiratory syncytial virus, the virus having an attenuated phenotype and comprising an M2-2 protein that comprises at least one mutated amino acid residue, in a physiologically acceptable carrier. In preferred embodiments, the immune response is a protective immune response. The vaccine can be administered in one or more doses to achieve the desired level of protection. The recombinant RSV is preferably administered to the upper respiratory tract (e.g., the nasopharynx) of the individual, and is preferably administered by spray, droplet or aerosol.

### Detection of Neutralizing Antibody

The measurement of serum anti-RSV neutralizing antibody against RSV infection from both A and B subgroups is very valuable for evaluating the efficacy of RSV vaccine candidates in recipients and for RSV seroepidemiological studies (Gonzalez et al. (2000) Vaccine 18:1763-1772).

Several methods have been described for the detection of RSV neutralizing antibody. These methods require pretreatment of virus with serial dilutions of antibody followed by infection of cell monolayers. The methods that have been used to detect residual RSV infectivity following virus neutralization by antibody include: reduced cytopathology (Beeler & van Wyke Coelingh (1989) J. Virol. 63:2941 -2950), reduction in plaque numbers (Coates et al. (1966) Am. J. Epidemiol. 83:299-313) or reduced RSV antigen expression (Anderson et al. (1985) Clin. Microbiol. 22:1050-1032). Each of the above methods can adequately detect RSV neutralizing antibody, however, most of these assays are labor intensive and/or the read-out is subjective. Such assays are not suited for the rapid screening and direct quantitation of a large number of samples.

The present invention provides recombinant RSVs containing the lacZ gene inserted in the rA2 and rA2-G_{B}F_{B} chimera, and their use in a rapid microneutralization assay to quantitate anti-RSV neutralizing antibody to subgroup A or subgroup B RSV. The methods and compositions of the invention utilize a previously described reverse genetics system for the expression of recombinant RSV, rA2, and a chimeric RSV (rA2-G_{B}F_{B}) encoding the G and F antigens of the RSV subgroup B 9320 strain in place of the A2 G and F antigens ((WO 02/44334); Cheng et al. (2001) Virology 283:59-68).

In brief, the lacZ can be inserted into recombinant RSVs expressing the G and F antigens derived from either RSV subgroup A or B. Host cells, such as HEp-2 cells infected with MVA-T7 and expressing N, P, and L, are transfected with the recombinant RSV cDNA incorporating lacZ (e.g., A-lacZ or B-lacZ, respectively). Following incubation, virus is recovered and amplified in fresh host cells, e.g., Vero cells. β-galactosidase is readily detectable in cells infected with either A-lacZ or B-lacZ by, e.g., Western blotting or by the colorimetric detection of enzyme activity. β-galactosidase enzyme activity reflects viral replication, and, therefore, can be used to measure virus infectivity after neutralization by serum anti-RSV neutralizing antibody.

Microneutralization is typically performed in a multiwell plate format, e.g., 96 well plates. For example, heat inactivated serum or plasma (56° C., 30 minutes) is serially diluted (2-fold) with medium containing 2% serum, e.g., OptiMEM/2% FBS) with or without guinea pig complement in a volume appropriate to the plate format, and A-lacZ or B-lacZ is added to each well and incubated. Approximately 50,000 Vero cells are added to the wells, and the plates are incubated under conditions suitable for virus replication. After an incubation period of between approximately 2 and 5 days, e.g., 3 days, the supernatant is removed, and the cells are washed with isotonic buffer, e.g., PBS, The cells, are incubated with lysis buffer, and enzymatic activity of β-galactosidase is measured by methods well known in the art. For example, β-galactosidase activity is favorably detected using a chromogenic substrate, chlorophenol red P-D-galactopyranoside (CPRG).

This microneutralization assay is rapid (3 days compared to 6 days for standard plaque reduction assays), less laborious, and suitable for automation using a variety of high-throughput assay systems (e.g., high-throughput robotic assay systems) and screening or testing of numerous samples. This microneutralization system can be readily adapted for assay of neutralizing antibodies for other viruses of family Paramyxoviridae by substituting appropriate recombinant virus constructs incorporating lacZ or another appropriate marker.

Significant heterotypic neutralizing antibodies are detected by the micro-neutralization assay of the invention, although higher neutralizing antibody titer is typically detected with virus containing homologous G and F proteins than that of the heterologous G and F proteins. Thus, the microneutralization assay of the invention can be used to distinguish antigenic variation between RSV strains contributed primarily by the G and F proteins of RSV.

The antibodies against the G and F proteins of RSV are typically long-lasting in vivo, whereas the antibodies against the internal proteins are of much shorter duration. (Connors et al. (1991) J. Virol. 65:1634-1637; Stott et al. (1987) J. Virol. 61:3855-3861). Detection of the long-lasting antibodies against the G and F proteins in human sera by the microneutralization assay makes this assay suitable, e.g., for sero-epidemiological surveys of RSV infection.

One aspect of the present invention provides methods of determining an antibody titer (e.g., to quantitate neutralizing antibodies). In the methods, a recombinant virus of family Paramyxoviridae and a sample comprising one or more antibodies are contacted in the presence of cells in which the virus can replicate. (Virus not neutralized by the antibodies can thus infect the cells.) Replication of the virus is permitted. The genome or antigenome of the recombinant virus comprises a marker, and the marker (e.g., presence and/or expression of the marker) is detected following viral replication.

In one class of embodiments, the recombinant virus comprises a respiratory syncytial virus (RSV). In preferred embodiments, the respiratory syncytial virus comprises a human respiratory syncytial virus of subgroup A (e.g., A-lacZ), subgroup B or a chimera thereof (e.g., a human RSV of subgroup A in which one or more proteins selected from the group consisting of the G glycoprotein and the F glycoprotein are replaced by one or more homologous proteins of a human RSV of subgroup B, e.g., B-lacZ).

In another class of embodiments, the recombinant virus comprises another virus of family Paramyxoviridae. For example, the recombinant virus can comprise a metapneumovirus, a sendai virus, a parainfluenza virus, a mumps virus, a newcastle disease virus, a measles virus, a canine distemper virus, or a rinderpest virus.

The sample comprising one or more antibodies can be derived from essentially any source and/or can be prepared or produced by essentially any means known in the art. For example, in one class of embodiments, the sample comprising one or more antibodies comprises a serum (e.g., a peripheral blood-derived serum), a bronchial lavage, or a nasal wash (e.g., serial dilutions of the serum, lavage, or wash).

The virus, sample comprising the antibodies, and the cells can be combined in various orders. Typically, contacting the recombinant virus and the sample in the presence of cells comprises combining the virus and the sample and then combining the combined virus and sample with the cells. In certain embodiments, the virus and the sample are contacted in the presence of one or more complement factors (e.g., complement components C1-C9). One of skill can determine experimentally whether or not addition of complement results in a reproducible and reasonable antibody titer (e.g., a titer consistent with the results of other currently accepted methods for quantitating neutralizing antibodies). For example, addition of complement results in a reasonable antibody titer in assays using RSV A-lacZ, but addition of complement appears to kill or otherwise inhibit RSV B-lacZ and thus does not result in a reasonable antibody titer in assays using B-lacZ.

The marker can comprise essentially any convenient marker. For example, the marker can comprise one or more of: a marker nucleic acid that encodes an optically detectable marker protein (e.g., a marker nucleic acid that encodes a beta galactosidase protein, a marker nucleic acid that encodes a green fluorescent protein, a marker nucleic acid that encodes a luciferase protein, or a marker nucleic acid that encodes a chloramphenicol transferase protein), a marker nucleic acid that encodes a selectable marker protein (e.g., a gene that confers cellular resistance to an antibiotic, e.g., a gene conferring resistance to neomycin) or a marker nucleic acid that is itself detectable. As mentioned previously, detecting the marker can comprise detecting the presence of anchor detecting expression of the marker. In certain embodiments, expression of the marker is quantitated (e.g., levels of a protein marker encoded by the nucleic acid marker can be quantitated). If necessary or desired, the cells can be washed and lysed prior to detecting expression of the marker.

Compositions, recombinant viruses, and nucleic acids related to the methods provide additional features of the invention. Thus, one general class of embodiments provides a composition comprising one or more antibodies and a recombinant virus of family Paramyxoviridae, the genome or antigenome of which comprises a marker. The recombinant virus can comprise a respiratory syncytial virus; for example, a human respiratory syncytial virus of subgroup A (e.g., A-lacZ), subgroup B or a chimera thereof (e.g., a human RSV of subgroup A in which one or more proteins selected from the group consisting of the G glycoprotein and the F glycoprotein, are replaced by one or more homologous proteins of a human RSV of subgroup B, e.g., B-lacZ). Alternatively, the recombinant virus can comprise another virus of family Paramyxoviridae, e.g., a metapneumovirus, a sendai virus, a parainfluenza virus, a mumps virus, a newcastle disease virus, a measles virus, a canine distemper virus, or a rinderpest virus.

The marker can comprise essentially any convenient marker. For example, the marker can comprise one or more of: a marker nucleic acid that encodes an optically detectable marker protein (e.g., a marker nucleic acid that encodes a beta galactosidase protein, a marker nucleic acid that encodes a green fluorescent protein, a marker nucleic acid that encodes a luciferase protein, or a marker nucleic acid that encodes a chloramphenicol transferase protein) or a marker nucleic acid that encodes a selectable marker-protein (e.g., a gene that confers cellular resistance to an antibiotic, e.g., a gene conferring resistance to neomycin).

The composition can further comprise cells in which the virus can replicate and/or one or more complement factors (e.g., one or more of complement components C1-C9).

Another class of embodiments provides a recombinant respiratory syncytial virus (RSV) comprising a genome or antigenome. The genome or antigenome comprises a marker, which marker comprises one or more of: a marker nucleic acid that encodes a beta galactosidase protein, a marker nucleic acid that encodes a luciferase protein, or a marker nucleic acid that encodes a selectable marker protein (e.g., a gene that confers cellular resistance to an antibiotic, e.g., a gene conferring resistance to neomycin). In certain embodiments, the recombinant RSV comprises a human RSV of subgroup A (e.g., A-lacZ), subgroup B or a chimera thereof (e.g., a human RSV of subgroup A in which one or more proteins selected from the group consisting of the G glycoprotein and the F glycoprotein are replaced by one or more homologous proteins of a human RSV of subgroup B, e.g., B-lacZ).

A related class of embodiments provides a nucleic acid encoding a recombinant RSV whose genome or antigenome comprises a marker, which marker comprises one or more of: a marker nucleic acid that encodes a beta galactosidase protein, a marker nucleic acid that encodes a luciferase protein, or a marker nucleic acid that, encodes a selectable marker protein (e.g., a gene that confers cellular resistance to an antibiotic, e.g., a gene conferring resistance to neomycin). The nucleic acid can be, e.g., a DNA (e.g., a cDNA) or an RNA. The nucleic acid can be an RSV genome or antigenome. A vector (e.g., a plasmid) can comprise the nucleic acid.

Another class of embodiments provides a recombinant virus of family Paramyxoviridae. The recombinant virus comprises a metapneumovirus, a sendai virus, a parainfluenza virus, a mumps virus, or a canine distemper virus. The virus comprises a genome, or antigenome comprising a marker, for example, one or more of: a nucleic acid that encodes an optically detectable marker protein (e.g., a marker nucleic acid that encodes a beta galactosidase protein, a marker nucleic acid that encodes a green fluorescent protein, a marker nucleic acid that encodes a luciferase protein, or a marker nucleic acid that encodes a chloramphenicol transferase protein) or a marker nucleic acid that encodes a selectable marker protein (e.g., a gene that confers cellular resistance to an antibiotic, e.g., a gene conferring resistance to neomycin).

A related class of embodiments provides a nucleic acid encoding a recombinant virus of family Paramyxoviridae, wherein the recombinant virus comprises a metapneumovirus, a sendai virus, a parainfluenza virus, a mumps virus, or a canine distemper virus and comprises a genome or antigenome comprising a marker. The nucleic acid can be, e.g., a DNA (e.g., a cDNA) or an RNA. The nucleic acid can be an RSV genome or antigenome. A vector (e.g., a plasmid) can comprise the nucleic acid.

### Kits

To facilitate use of the RSV vectors and vector systems of the invention, any of the vectors, e.g., chimeric RSV virus vectors, RSV vectors incorporating lacZ encoding polynucleotides, variant RSV polypeptide plasmids, RSV polypeptide library plasmids, etc., and additional components, such as, buffer, cells, culture medium, useful for producing recombinant RSV, can be packaged in the form of a kit. Typically, the kit contains, in addition to the above components, additional materials which can include, e.g., instructions for performing the methods of the invention, packaging material, and a container.

In addition, kits for detecting neutralizing antibodies using the microneutralization assay of the invention are a feature of the invention. Typically such kits include one or more recombinant viruses of family Paramyxoviridae (e.g., one or more recombinant RSV constructs, e.g., A-lacZ, B-lacZ, rA2 or rA2-G_{B}F_{B}), and optionally contain such additional components as assay substrates, such as a colorimetric or fluorogenic substrate of β-galactosidase, control serum, buffer, cells, culture medium, and the like. Additionally, the kit typically contains materials such as instructions, packaging material, a container, etc.

### Family Paramyxoviridae

Virus families containing enveloped single-stranded RNA of the negative-sense genome are classified into groups having non-segmented genomes (e.g., Paramyxoviridae, Rhabdoviridae) or those having segmented genomes (e.g., Orthormyxoviridea, Bunyaviridae, Arenaviridae). Viruses of family Paramyxoviridae have been classified into two subfamilies and several genera (e.g., as described in the Universal Virus Database of the International Committee of Taxonomy of Viruses, www.ncbi.nlm.nih.gov/ICTVdb). Subfamily Paramyxovirinae includes the Respirovirus genus (e.g., Sendai virus, bovine parainfluenza virus 3, human parainfluenza viruses 1 and 3, simian virus 10), the Rubulaviros genus (e.g., mumps virus, human parainfluenza viruses 2 and 4, Mapuera virus, porcine rubolavirus, La-Piedad-Michoacan-Mexico virus, simian parainfluenza virus 5), the Morbillivirus genus (e.g., measles virus, canine distemper virus, cetacean morbillivirus, Edmonston virus, Peste-des-petits-ruminants virus, Rinderpest virus), the Henipavirus genus (e.g., Hendra virus, Nipah virus), the Avulavirus genus (Newcastle disease virus, avian parainfluenza viruses 1-9), and the “TPMV-like viruses” genus (e.g., Tupaia virus). Subfamily Pneumovirinae includes the Pneumovirus genus (e.g., murine pneumonia virus, bovine RSV, human RSV (e.g., subgroups A2, B1, S2)) and the Metapneumovirus genus (e.g., Turkey rhinotracheitis virus). The family also includes Fer-de-Lance virus and Nariva virus.

Negative strand RNA viruses can be genetically engineered and recovered using a recombinant reverse genetics approach (U.S. Pat. No. 5,166,05 to Palese et al.). Although this method was originally applied to engineer influenza viral genomes (Luytjes et al. 1989, Cell 59:1107-1113; Enami et al., 1990, Proc. Natl. Acad. Sci. USA 92:11563-11567), it has been successfully applied to a wide variety of segmented and nonsegmented negative strand RNA viruses, e.g., rabies (Schnell et al. 1994, EMBO J. 13:4195-4203); VSV (Lawson et al 1995, Proc Natl. Acad. Sci. USA 92: 4477-4481); measles virus (Radecke et al. 1995, EMBO J. 14:5773-5784); rinderpest virus (Baron & Barrett, 1997, J. Virol. 71: 1265-1271); human parainfluenza virus (Hoffman &. Banerjee, 1997, J. Virol. 71; 3272-3277; Dubin et al., 1997, Virology 235:323-332); SV5 (He et al., 1997, Virology 237:249-260); canine distemper virus (Gassen et al., 2000, J. Virol. 74:10737-44); and Sendai virus (Park et al., 1991, Proc. Natl. Acad. Sci. USA 88: 5537-5541; Kato et al., 1996, Genes to Cells 1:569-579). Rescue of RSV has been described e.g., in Collins et al., 1991, Proc. Natl. Acad. Sci. USA 88: 9663-9667; Jin et al. (1998) Virology 251:206-214; and WO 02/44334 by Jin et al., entitled “Recombinant RSV virus expression systems and vaccines,” and is briefly described herein. (See also e.g., Jin et al. (2000) J. Virol 74:74-82; Jin et al. (2000) Virology 273:210-218; Cheng et al. (2001) Virology 283:59-68; and Tang et al. (2001) J. Virol. 75:11328-11335.) Methods for propagation, separation from host cell cellular components, and/or further purification of viruses of family Paramyxoviridae are well known to those skilled in the art.

### Cell Culture

Typically, propagation of a recombinant virus (e.g., recombinant RSV) is accomplished in the media compositions in which the host cell is commonly cultured. Suitable host cells for the replication of RSV include, e.g., Vero cells, HEp-2 cells. Typically, cells are cultured in a standard commercial culture medium, such as Dulbecco's modified Eagle's medium supplemented with serum (e.g., 10% fetal bovine serum), or in serum free medium, under controlled humidity and CO₂ concentration suitable for maintaining neutral buffered pH (e.g., at pH between 7.0 and 7.2). Optionally, the medium contains antibiotics to prevent bacterial growth, e.g., penicillin, streptomycin, etc., and/or additional nutrients, such as L-glutamine, sodium pyruvate, non-essential amino acids, additional supplements to promote favorable growth characteristics, e.g., trypsin, β-mercaptoethanol, and the like.

Procedures for maintaining mammalian cells in culture have been extensively reported, and are known to those of skill in the art. General protocols are provided, e.g., in Freshney (1983) Culture of Animal Cells: Manual of Basic Technique, Alan R. Liss, New York; Paul (1975) Cell and Tissue Culture, 5^{th} ed., Livingston, Edinburgh; Adams (1980) Laboratory Techniques in Biochemistry and Molecular Biology-Cell Culture for Biochemists. Work and Bunion (eds.) Elsevier, Amsterdam. Additionally, variations in such procedures adapted to the present invention are readily determined through routine experimentation.

Cells for production of RSV can be cultured in serum-containing or serum free medium. In some cases, e.g., for the preparation of purified viruses, it is desirable to grow the host cells in serum free conditions. Cells can be cultured in small scale, e.g., less than 25 ml medium, culture tubes or flasks or in large flasks with agitation, in rotator bottles, or on microcarrier beads (e.g., DEAE-Dextran microcarrier beads, such as Dormacell, Pfeifer & Langen; Superbead, Flow Laboratories; styrene copolymer-tri-methylamine beads, such as Hillex, SoloHill, Ann Arbor) in flasks, bottles or reactor cultures. Microcarrier beads are small spheres (in the range of 100-200 microns in diameter) that provide a large surface area for adherent cell growth per volume of cell culture. For example a single liter of medium can include more than 20 million microcarrier beads providing greater than 8000 square centimeters of growth surface. For commercial production of viruses, e.g., for vaccine production, it is often desirable to culture the cells in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); laboratory and commercial scale bioreactors from B. Braun Biotech International (B. Braun Biotech, Melsungen, Germany).

Other useful references, e.g., for cell isolation and culture (e.g., of bacterial cells containing recombinant nucleic acids, e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

### Introduction of Vectors into Host Cells

Vectors, e.g., vectors incorporating RSV polynucleotides, are introduced (e.g., transfected) into host cells according to methods well known in the art for introducing heterologous nucleic acids into eukaryotic cells, including, e.g., calcium phosphate co-precipitation, electroporation, microinjection, lipofection, and transfection employing polyamine transfection reagents. For example, vectors, e.g., plasmids, can be transfected into host cells, e.g., Vero cells or Hep-2 cells, using the transfection reagent LipofectACE or Lipofectamine 2000 (Invitrogen) according to the manufacturer's instructions. Alternatively, electroporation can be employed to introduce vectors incorporating RSV genome segments into host cells.

### Model Systems

Attenuated RSV, e.g. those described herein, can be tested in in vitro and in vivo models to confirm adequate attenuation, genetic stability, and/or immunogenicity for vaccine use. In in vitro assays, e.g., replication in cultured cells, the virus can be tested, e.g., for genetic stability, temperature sensitivity of virus replication and/or a small plaque phenotype. RSV can be further tested in animal models of infection. A variety of animal models, e.g., primate (e.g., chimpanzee, African green monkey) and rodent (e.g., cotton rat), are known in the art, as described briefly herein and in U.S. Pat. No. 5,922,326 to Murphy et al. (Jul. 13, 1999) entitled “Attenuated respiratory syncytial virus compositions”; U.S. Pat. No. 4,800,078; Meignier et al., eds., Animal Models of Respiratory Syncytial Virus Infection, Merieux Foundation Publication, (1991); Prince et al., Virus Res. 3:193-206 (1985); Richardson et al., J. Med. Virol. 3:91-100 (1978); Wright et al., Infect. Immun., 37:397-400 (1982); and Crowe et al., Vaccine 11:1395-1404 (1993).

### Methods And Compositions For Prophylactic Administration of Vaccines

Typically, the attenuated recombinant RSV of this invention as used in a vaccine is sufficiently attenuated such that symptoms of infection, or at least symptoms of serious infection, will not occur in most individuals immunized (or otherwise infected) with the attenuated RSV. In embodiments in which viral components (e.g., the nucleic acids or proteins herein) are used as a vaccine or as immunogenic components of a vaccine, serious infection is not typically an issue. In some instances, the attenuated RSV (or the RSV components of the invention) can still be capable of producing symptoms of mild illness (e.g., mild upper respiratory illness) and/or of dissemination to unvaccinated individuals. However, virulence is sufficiently abrogated such that severe lower respiratory tract infections do not typically occur in the vaccinated or incidental host.

Recombinant RSV, including, e.g., chimeric RSV, and/or RSV components of the invention can be administered prophylactically in an appropriate carrier or excipient to stimulate an immune response, e.g., one which is specific for one or more strains of RSV. Typically, the carrier or excipient is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions insuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, oral, topical, etc. The resulting aqueous solutions can e.g., be packaged for use as is or lyophilized, the lyophilized preparation being combined with a sterile solution prior to administration

Generally, the RSV or RSV components of the invention are administered in a quantity sufficient to stimulate an immune response specific for one or more strains of RSV (e.g., an immunologically effective amount of RSV or RSV component is administered). Preferably, administration of RSV or RSV component(s) elicits a protective immune response. Dosages and methods for eliciting a protective anti-viral immune response, adaptable to producing a protective immune response against RSV are known to those of skill in the art. See, e.g., U.S. Pat. No. 5,922,326; Wright et al., Infect. Immun. 37:397-400 (1982); Kim et al., Pediatrics 52:56-63 (1973); and Wright et al., J. Pediatr. 88:931-936 (1976). For example, virus can be provided in the range of about 10³-10⁶ pfu (plaque forming units) per dose administered (e.g., 10⁴-10⁵ pfu per dose administered). Typically, the dose will be adjusted based on, e.g., age, physical condition, body weight, sex, diet, mode and time of administration, and other clinical factors. The prophylactic vaccine formulation can be systemically administered, e.g., by subcutaneous or intramuscular injection using a needle and syringe or a needleless injection device. Preferably, the vaccine formulation is administered intranasally, e.g., by drops, aerosol (e.g., large particle aerosol (greater than about 10 microns)), or spray into the upper respiratory tract. While any of the above routes of delivery results in a protective systemic immune response, intranasal administration confers the added benefit of eliciting mucosal immunity at the site of entry of the virus. For intranasal administration, attenuated live virus vaccines are often preferred, e.g., an attenuated, cold adapted and/or temperature sensitive recombinant RSV, e.g., a chimeric recombinant RSV. RSV components as described herein can also be used.

While stimulation of a protective immune response with a single dose is preferred, additional dosages can be administered, by the same or different route, to achieve the desired prophylactic effect. In neonates and infants, for example, multiple administrations may be required to elicit sufficient levels of immunity. Administration can continue at intervals throughout childhood, as necessary to maintain sufficient levels of protection against wild-type RSV infection. Similarly, adults who are particularly susceptible to repeated or serious RSV infection, such as, for example, health care workers, day care workers, family members of young children, elderly, individuals with compromised cardiopulmonary function, etc. may require multiple immunizations to establish and/or maintain protective immune responses. Levels of induced immunity can be monitored, for example, by measuring amounts of neutralizing secretory and serum antibodies, and dosages adjusted or vaccinations repeated as necessary to maintain desired levels of protection.

Alternatively, an immune response can be stimulated by ex vivo or in vivo targeting of dendritic cells with virus. For example, proliferating dendritic cells are exposed to viruses in a sufficient amount and for a sufficient period of time to permit capture of the RSV antigens by the dendritic cells. The cells are then transferred into a subject to be vaccinated by standard intravenous transplantation methods.

Optionally, the formulation for prophylactic administration of the RSV also contains one or more adjuvants for enhancing the immune response to the RSV antigens. Suitable adjuvants include, for example: complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvant QS-21.

If desired, prophylactic vaccine administration of RSV can be performed in conjunction with administration of one or more immunostimulatory molecules. Immunostimulatory molecules include various cytokines, lymphokines and chemokines with immuuostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the RSV, or can be administered separately. Either the protein or, an expression vector encoding the protein can be administered to produce an immunostimulatory effect.

Although vaccination of an individual with an attenuated RSV of a particular strain of a particular subgroup can induce cross-protection against RSV of different strains and/or subgroups, cross-protection can be enhanced, if desired, by vaccinating the individual with attenuated RSV from at least two strains, e.g., each of which represents a different subgroup. Similarly, the attenuated RSV vaccines of this invention can optionally be combined with vaccines that induce protective immune responses against other infectious agents.

### Production of Viral Nucleic Acids

In the context of the invention, viral (e.g., RSV) nucleic acids and/or proteins are manipulated according to well known molecular biology techniques. Detailed protocols for numerous such procedures, including amplification, cloning, mutagenesis, transformation, and the like, are described in, e.g., in Ausubel et al. Current Protocols in Molecular Biology (supplemented through 2003) John Wiley & Sons, New York (“Ausubel”); Sambrook et al. Molecular Cloning—A Laboratory Manual (3rd Ed.), Vol. 1-3, Cold Spring Harbor Laboratory, Cold Spring Harbor, N.Y., 2001 (“Sambrook”), and Berger and Kimmel Guide to Molecular Cloning Techniques, Methods in Enzymology volume 152 Academic Press, Inc., San Diego, Calif. (“Berger”).

In addition to the above references, protocols for in vitro amplification techniques, such as the polymerase chain reaction (PCR), the ligase chain reaction (LCR), Qú-replicase amplification, and other RNA polymerase mediated techniques (e.g., NASBA), useful e.g., for amplifying cDNA polynucleotides of the invention, are found in Mullis et al. (1987) U.S. Pat. No. 4,683,202; PCR Protocol A Guide to Methods and Applications (Innis et al. eds) Academic Press inc. San Diego, Calif. (1990) (“Innis”); Amheim and Levinson (1990) C&EN 36; The Journal of NIH Research (1991) 3:81; Kwoh et al. (1989) Proc Natl Acad Sci USA, 86, 1173; Guatelli et al. (1990) Proc Natl Acad Sci USA 87:1874; Lomell et al. (1989) J. Clin Chem 35:1826; Landegren et al. (1988) Science 241:1077; Van Brunt (1990) Biotechnology 8:291; Wu and Wallace (1989) Gene 4:560; Barringer et al. (1990) Gene 89:117, and Sooknanan and Malek (1995) Biotechnology 13:563. Additional methods, useful for cloning nucleic acids in the context of the present invention, include Wallace et al. U.S. Pat. No. 5,426,039. Improved methods of amplifying large nucleic acids by PCR are summarized in Cheng et al. (1994) Nature 369:684 and the references therein.

Certain polynucleotides of the invention, e.g., oligonucleotides, can be synthesized utilizing various solid-phase strategies including mononucleotide- and/or trinucleotide-based phospboramidite coupling chemistry. For example, nucleic acid sequences can be synthesized by the sequential addition of activated monomers and/or trimers to an elongating polynucleotide chain. See e.g., Caruthers, M. H. et al. (1992) Meth Enzymol 211:3.

In lieu of synthesizing the desired sequences, essentially any nucleic acid can be custom ordered from any of a variety of commercial sources, such as The Midland Certified Reagent Company (mcrc@oligos.com), The Great American Gene Company (www.genco.com), ExpressGen, Inc. (www.expressgen.com), Operon Technologies, Inc. (www.operon.com), and many others.

In addition, substitutions of selected amino acid residues in viral polypeptides can be accomplished by, e.g., site directed mutagenesis. For example, viral polypeptides with amino acid substitutions functionally correlated with desirable phenotypic characteristic, e.g., an attenuated phenotype, cold adaptation, temperature sensitivity, can be produced by introducing specific mutations into a viral nucleic acid segment (e.g., a cDNA) encoding the polypeptide. Methods for site directed mutagenesis are well known in the art, and described, e.g., in Ausubel, Sambrook, and Berger, supra. Numerous kits for performing site directed mutagenesis are commercially available, e.g., the Chameleon Site Directed Mutagenesis Kit (Stratagene, La Jolla), and can be used according to the manufacturers instructions to introduce, e.g., one or more nucleotide substitutions specifying one or more amino acid substitutions into an RSV polynucleotide.

Various types of mutagenesis are optionally used in the present invention, e.g., to modify nucleic acids and encoded polypeptides and/or viruses to produce conservative or non-conservative variants (e.g., to introduce an amino acid substitution, insertion or deletion into an RSV P, M2-1 and/or M2-2 protein). Any available mutagenesis procedure can be used. Such mutagenesis procedures optionally include selection of mutant nucleic acids and polypeptides for one or more activity of interest. Procedures that can be used include, but are not limited to: site-directed point mutatgenesis, random point mutagenesis, in vitro or in vivo homologous recombination (DNA shuffling), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, and many others known to persons of skill. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like. In another class of embodiments, modification is essentially random (e.g., as in classical DNA shuffling).

Several of these procedures are set forth in Sambrook and Ausubel, herein. Additional information on these procedures is found in the following publications and the references cited therein: Arnold, Protein engineering for unusual environments, Current Opinion in Biotechnology 4:450-455 (1993); Bass et al., Mutant Trp repressors with new DNA-binding specificities, Science 242:240-245. (1988); Botstein & Shortle, Strategies and applications of in vitro mutagenesis, Science 229:1193-1201(1985); Carter et al., Improved oligonucleotide site-directed mutagenesis using M13 vectors, Nucl. Acids Res. 13: 4431-4443 (1985); Carter, Site-directed mutagenesis, Biochem. J. 237:1-7 (1986); Carter, Improved oligonucleotide-directed mutagenesis using M13 vectors, Methods in Enzymol. 154; 382-403 (1987); Dale et al., Oligonucleotide-directed random mutagenesis using the phosphorothioate method, Methods Mol. Biol. 57:369-374 (1996); Eghtedarzadeh & Henikoff, Use of oligonucleotides to generate large deletions, Nucl. Acids Res. 14: 5115 (1986); Fritz et al., Oligonucleotide-directed construction of mutations: a gapped duplex DNA procedure without enzymatic reactions in vitro, Nucl. Acids Res. 16: 6987-6999 (1988); Grundström et al., Oligonucleotide-directed mutagenesis by microscale ‘shot-gun’ gene synthesis, Nucl. Acids Res. 13: 3305-3316 (1985); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel, Rapid and efficient site-specific mutagenesis without phenotypic selection, Proc. Natl. Acad. Sci. USA 83:488-492 (1985); Kunkel et al., Rapid and efficient site-specific mutagenesis without phenotypic selection, Methods in Enzymol. 154, 367-382 (1987); Kramer et al., The gapped duplex DNA approach to oligonucleotide-directed mutation construction, Nucl. Acids Res., 12: 9441-9456 (1984); Kramer & Fritz Oligonucleotide-directed construction of mutations via gapped duplex DNA, Methods in Enzymol. 154:350-367 (1987); Kramer et al., Point Mismatch Repair, Cell 38:879-887 (1984); Kramer et al., Improved enzymatic in vitro reactions in the gapped duplex DNA approach to oligonucleotide-directed construction of mutations, Nucl. Acids Res. 16: 7207 (1988); Ling et al., Approaches to DNA mutagenesis: on overview, Anal Biochem. 254(2): 157-178 (1997); Lorimer and Pastan Nucleic Acids Res. 23, 3067-8 (1995); Mandecki, Oligonucleotide-directed double-strand break repair plasmids of Escherichia coli: a method for site-specific mutagenesis, Prop. Natl. Acad. Sci. USA, 83:7177-7181 (1986); Nakamaye & Eckstein, Inhibition of restriction endonuclease Nci I cleavage by phosphorothioate groups and its application to oligonucleotide-directed mutagenesis, Nucl. Acids Res. 14; 9679-9698 (1986); Nambiar et al., Total synthesis and cloning of a gene coding for the ribonuclease S protein, Science 223: 1299-1301 (1984); Sakamar and Khorana, Total synthesis and expression of a gene for the a-subunit of bovine rod outer segment guanine nucleotide-binding protein (transducin), Nucl. Acids Res. 14; 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Sieber, et al., Nature Biotechnology, 19:456-460 (2001); Smith, In vitro mutagenesis, Ann. Rev. Genet. 19:423-462(1985); Methods In Enzymol. 100: 468-500 (1983); Methods in Enzymol, 154: 329-350 (1987); Stemmer, Nature 370, 389-91 (1994); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Importance of hydrogen-bond formation in stabilizing the transition state of subtilisin, Phil. Trans. R. Soc. Lond. A 317: 415-423 (1986); Wells et al., Cassette mutagenesis: an efficient method for generation of multiple mutations at defined sites, Gene 34:315-323 (1985); Zoller & Smith, Oligonucleotide-directed mutagenesis using M13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); and Zoller & Smith, Oligonucleotide-directed mutagenesis: a simple method using two oligonucleotide primers and a single-stranded DNA template, Methods m Enzymol. 154:329-350 (1987). Additional details on many of the above methods can be found in Methods in Enzymology Volume 154, which also describes useful controls for trouble-shooting problems with various mutagenesis methods.

### Sequence Variations

Silent Variations

Due to the degeneracy of the genetic code, any of a variety of nucleic acids sequences encoding polypeptides and/or viruses of the invention are optionally produced, some which can bear lower levels of sequence identity to the RSV nucleic acid and polypeptide sequences in the figures. The following provides a typical codon table specifying the generic code, found in many biology and biochemistry texts.

TABLE 1
Codon Table

| Amino acids | | | Codon | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

The codon table shows that many amino acids are encoded by more than one codon. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

As an example, the nucleic acid sequence corresponding to residues 175-177 of the RSV A2 phosphoprotein (EEM) is GAAGAAATG. A silent variation of this sequence includes GAGGAGATG (also encoding EEM).

Such “silent variations” are one species of “conservatively modified variations”, discussed below. One of skill will recognize that each codon in a nucleic acid (except ATG, which is ordinarily the only codon for methionine) can be modified by standard techniques to encode a functionally identical polypeptide. Accordingly, each silent variation of a nucleic acid which encodes a polypeptide is implicit in any described sequence. The invention, therefore, explicitly provides each and every possible variation of a nucleic acid sequence encoding a polypeptide of the invention that could be made by selecting combinations based on possible codon choices. These combinations are made in accordance with the standard triplet genetic code (e.g., as set forth in Table 1, or as is commonly available in the art) as applied to the nucleic acid sequence encoding an RSV polypeptide of the invention. All such variations of every nucleic acid herein are specifically provided and described by consideration of the sequence in combination with the genetic code. One of skill is fully able to make these silent substitutions using the methods herein.

### Conservative Variations

“Conservatively modified variations” or, simply, “conservative variations” of a particular nucleic acid sequence or polypeptide are those which encode identical or essentially identical amino acid sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

Conservative substitution tables providing functionally similar amino acids are well known in the art. Table 2 sets forth six groups which contain amino acids that are “conservative substitutions” for one another.

TABLE 2
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Thus, “conservatively substituted variations” of a polypeptide sequence of the present invention include substitutions of a small percentage, typically less than 5%, more typically less than 2% or 1%, of the amino acids of the polypeptide sequence, with a conservatively selected amino acid of the same conservative substitution group.

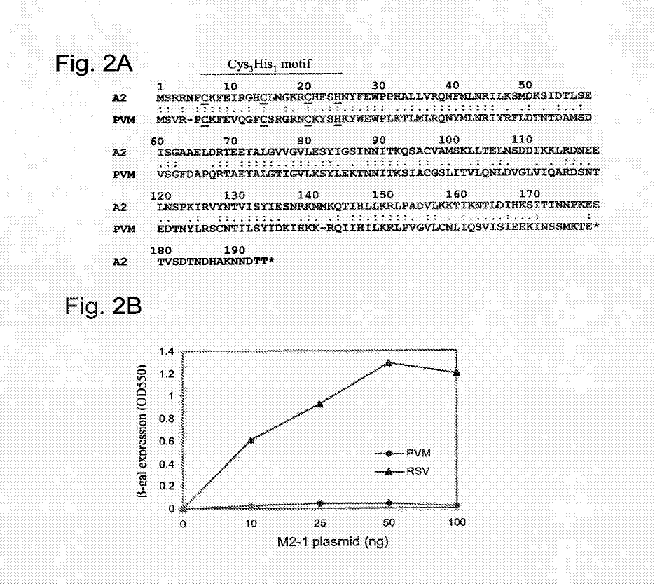

For example, a conservatively substituted variation of the RSV strain A2 M2-1 polypeptide in FIG. 2A will contain “conservative substitutions”, according to the six groups defined above, in up to about 10 residues (i.e., about 5% of the amino acids) in the full-length polypeptide.

In a further example, if conservative substitutions were localized in the region corresponding to amino acids 171-176 of RSV A2 P (IGLREE, SEQ ID NO:80), examples of conservatively substituted variations of this region include conservative substitutions of VGIKDD (SEQ ID NO:81) or IGVKDE (SEQ ID NO:82) (or any others that can be made according to Table 2) for IGLREE. Listing of a protein sequence herein, in conjunction with the above substitution table, provides an express listing of all conservatively substituted proteins.

Finally, the addition or deletion of sequences which do not alter the encoded activity of a nucleic acid molecule, such as the addition or deletion of a non-functional sequence, is a conservative variation of the basic nucleic acid or polypeptide.

One of skill will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed or claimed virus, nucleic acid or protein are a feature of the present invention.

### Defining Nucleic Acids by Hybridization

Nucleic acids of the invention can optionally be identified by hybridization. That is, nucleic acids of the invention can include a first nucleic acid that selectively hybridizes to a second nucleic acid encoding an artificially mutated or chimeric P, M2-1 or M2-2 protein of the invention (or complement thereof) under stringent conditions with at least five times the affinity that it hybridizes to a third, parental nucleic acid that was artificially mutated to produce the second nucleic acid.

“Selectively hybridizing” or “selective hybridization” includes hybridization, under stringent hybridization conditions, of a nucleic acid sequence to a specified nucleic acid target sequence to a detectably greater degree that its hybridization to non-target nucleic acid sequences. Selectively hybridizing sequences have at least 50%, or 60% or 70% or 80% or 90% sequence identity or more, e.g., preferably 95% sequence identity, and most preferably 98-100% sequence identity (i.e., complementarity) with each other.

“Stringent hybridization” conditions or “stringent conditions” in the context of nucleic acid hybridization assay formats are sequence dependent, and are different under different environmental parameters. An extensive guide to hybridization of nucleic acids is found in Tijssen (1993) Laboratory Techniques in Biochemistry and Molecular Biology—Hybridization with Nucleic Acid Probes Part 1, Chapter 2 “Overview of Principles of Hybridization and the Strategy of Nucleic Acid Probe Assays” Elsevier, New York. Generally, highly stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ point for a particular nucleic acid of the present invention. Stringent hybridization conditions are sequence-dependent and will be different in different circumstances. Longer sequences hybridize specifically at higher temperatures.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent-wash conditions is a 0.2×SSC wash at 65° C. for 15 minutes (see, Sambrook, supra for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher, e.g., 5×, 10×, 20×, 50×, 100% or more) than that observed for control probe in the particular hybridization assay indicates detection of a specific hybridization. For example, the control probe can be the third, parental nucleic acid, as noted above. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

Nucleic acids that selectively hybridize to a nucleic acid encoding an RSV of the invention (e.g., an attenuated RSV comprising an artificially mutated and/or chimeric P, M2-1 and/or M2-2 protein) under stringent conditions with at least five times the affinity that they hybridize to, e.g., a nucleic acid encoding a wild-type RSV are thus features of the invention. Similarly, nucleic acids that selectively hybridize to a nucleic acid encoding a polypeptide of the invention (e.g., an artificially mutated or chimeric P, M2-1 or M2-2 protein, or portion thereof) under stringent conditions with at least five times the affinity that they hybridize to, e.g., a nucleic acid encoding a wild-type P, M2-1 or M2-2 protein are also features of the invention.

### Defining Proteins by Immunoreactivity

Because the polypeptides of the invention provide a variety of new polypeptide sequences, the polypeptides also provide new structural features which can be recognized, e.g., in immunological assays. The generation of antisera which specifically bind the polypeptides of the invention, as well as the polypeptides which are bound by such antisera, are a feature of the invention.

Thus, the proteins of the invention can also be identified by immunoreactivity; e.g., the proteins of the invention can include an amino acid sequence or subsequence that is specifically bound by an antibody that specifically binds an artificially mutated (or chimeric) P, M2-1 or M2-2 protein of the invention but that does not bind the parental P, M2-1 or M2-2 protein that was altered to produce the artificially mutated (or chimeric) P, M2-1 or M2-2 protein.

Methods of producing antibodies, performing immunoassays, and the like are well known in the art. See e.g., Harlow and Lane (1988) Antibodies, A Laboratory Manual, Cold Spring Harbor Publications, New York.

In one typical format, the immunoassay uses a polyclonal antiserum which was raised against one or more polypeptides corresponding to one or more of the artificially mutated and/or chimeric P, M2-1 or M2-2 proteins of the invention, or a substantial subsequence thereof (i.e., at least about 30%, 40%, 50%, 60%, 70%, 80%, 90%, 95%, or 98% or more of one of the full length P, M2-1 or M2-2 proteins of the invention). The full set of potential polypeptide immunogens derived from one or more of the P, M2-1 or M2-2 proteins of the invention are collectively referred to below as “the immunogenic polypeptides.” The resulting antisera is optionally selected to have low cross-reactivity against the control wild-type P, M2-1 or M2-2 polypeptides and/or other known mutant or chimeric P, M2-1 or M2-2 polypeptides, and any such cross-reactivity is removed by immunoabsorption with one or more of the control P, M2-1 or M2-2 polypeptides, prior to use of the polyclonal antiserum in the immunoassay.

In order to produce antisera for use in an immunoassay, one or more of the immunogenic polypeptides is produced and purified as described herein. For example, recombinant protein may be-produced in a mammalian cell line. An inbred strain of mice (used in this assay because results are more reproducible due to the virtual genetic identity of the mice) is immunized with the immunogenic polypeptide(s) in combination with a standard adjuvant, such as Freund's adjuvant, and a standard mouse immunization protocol (see Harlow and Lane (1988) Antibodies, A Laboratory Manual, Cold Spring Harbor Publications, New York, for a standard description of antibody generation, immunoassay formats and conditions that can be used to determine specific immunoreactivity). Alternatively, one or more synthetic or recombinant polypeptides derived from the sequences disclosed herein is conjugated to a carrier protein and used as an immunogen.

Polyclonal sera are collected and titered against the immunogenic polypeptide(s) in an immunoassay, for example, a solid phase immunoassay with one or more of the immunogenic polypeptides immobilized on a solid support. Polyclonal antisera with a titer of 10⁶ or greater are selected, pooled and subtracted with the control P, M2-1 or M2-2 polypeptides to produce subtracted pooled titered polyclonal antisera,

The subtracted pooled titered polyclonal antisera are tested for cross reactivity against the control P, M2-1 or M2-2 polypeptides. Preferably at least two of the immunogenic P, M2-1 or M2-2 polypeptides are used in this determination, preferably in conjunction with at least two of the control P, M2-1 or M2-2 polypeptides, to identify antibodies which are specifically bound by the immunogenic polypeptide(s).

In this comparative assay, discriminatory binding conditions are determined for the subtracted titered polyclonal antisera which result in at least about a 5-10 fold higher signal to noise ratio for binding of the titered polyclonal antisera to the immunogenic P, M2-1 or M2-2 polypeptides as compared to binding to the control P, M2-1 or M2-2 polypeptides. That is, the stringency of the binding reaction is adjusted by the addition of non-specific competitors, such as albumin or non-fat dry milk, or by adjusting salt conditions, temperature, or the like. These binding conditions are used in subsequent assays for determining whether a test polypeptide is specifically bound by the pooled subtracted polyclonal antisera. In particular, a test polypeptide which shows at least a 2-5× higher signal to noise ratio than the control polypeptides under discriminatory binding conditions, and at least about a ½ signal to noise ratio as compared to the immunogenic polypeptide(s), shares substantial structural similarity or homology with the immunogenic-polypeptide(s) as compared to the control polypeptides, and is, therefore, a polypeptide of the invention.

In another example, immunoassays in the competitive binding format are used for detection of a test polypeptide. For example, as noted, cross-reacting antibodies are removed from the pooled antisera mixture by immunoabsorption with the control P, M2-1 or M2-2 polypeptides. The immunogenic polypeptide(s) are then immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein (s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control proteins to compete for binding to the pooled subtracted antisera is determined as compared to the ability of the immunogenic polypeptide(s) to compete for binding to the antisera. Again, the percent cross-reactivity for the control polypeptides is calculated, using standard calculations. Where the percent cross-reactivity is at least 5-10× as high for the test polypeptides, the test polypeptides are said to specifically bind the pooled subtracted antisera, and are, therefore, polypeptides of the invention.

In general, the immunoabsorbed and pooled antisera can be used in a competitive binding immunoassay as described herein to compare any test polypeptide to the immunogenic polypeptide(s). In order to make this comparison, the two polypeptides are each assayed at a wide range of concentrations and the amount of each polypeptide required to inhibit 50% of the binding of the subtracted antisera to the immobilized protein is determined using standard techniques. If the amount of the test polypeptide required is less than twice the amount of the immunogenic polypeptide that is required, then the test polypeptide is said to specifically bind to an antibody generated to the immunogenic polypeptide, provided the amount is at least about 5-10× as high as for a control polypeptide.

As a final determination of specificity, the pooled antisera is optionally fully immunosorbent with the immunogenic polypeptide(s) (rather than the control polypeptides) until little or no binding of the resulting immunogenic polypeptide subtracted pooled antisera to the immunogenic polypeptide(s) used in the immunoabsorption is detectable. This fully immonosorbed antisera is then tested for reactivity with the test polypeptide. If little or no reactivity is observed (i.e., no more than 2× the signal to noise ratio observed for binding of the fully immunosorbent antisera to the immunogenic polypeptide), then the test polypeptide is specifically bound by the antisera elicited by the immunogenic protein.

### Purification Methods

In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protcols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Functional Mutations in the M2-1 Protein of RSV

In contrast to RSV M2-1, PVM M2-1 has a very low level of activity in promoting transcriptional processivity. To characterize the basis of this difference, two chimeric proteins were constructed between the M2-1 protein encoding sequences of respiratory syncytial virus (RSV) and pneumovirus of mouse (PVM): 1) the PR (PV/RS) chimera including the N-terminal 29 amino acids from PVM and the remaining C-terminal 164 amino acids from RSV, and 2) the RP (RS/PV) chimera including the N-terminal 30 amino acids from RSV and the remaining C-terminus from PVM. Transcriptional activity was assayed in an RSVlacZ minigenome assay. Additionally, mutagenesis was performed in the PR M2-1 chimera cDNA to change the PVM residues to those of RSV.

### Materials and Methods

### Cells and Viruses

Monolayers of HEp-2 cells were maintained in DMEM supplemented with 10% fetal bovine serum. Modified vaccinia virus Ankara (MVA) expressing T7 RNA polymerase, MVA-T7, was obtained from Dr. Bernard Moss (Sutter et al. 1995, Febs Lett 371:9-12; and Wyatt et al. 1995, Virology 210:202-205) and propagated in CEK cells (SPAFAS).

### Construction of M2-1 Mutants

The protein expression plasmids encoding the N, P, L or M2-1 gene under the control of the T7 promoter in pCITE2a vector (Novagen) were described previously (Tang et al. (2001) J. Virol, 75:11328-11335, and in WO 02/44334, which are incorporated herein in their entirety for all purposes). The PVM M2-1 gene was amplified from PVM M2-1 cDNA (Ahmadian Easton (1999) J. Gen Virol. 80:2011-2016) using primers of 5′BsmBI (gcatcgtctcccatgagtgtgagaccttgc; SEQ ID NO:1) and 3′BamHI (ctcgagctgcagggatccg; SEQ ID NO:2) and cloned into the NcoI site of pCITE2a. To construct chimeric M2-1 plasmids, an MscI restriction enzyme site that is present in RSV M2-1 at nt 7693 was introduced into the corresponding position of pPVM-M2-1 using the Quick Change Site-directed Mutagenesis Kit (Stratagene). RP M2-1 was constructed by fusing the RSV N-terminal 30 amino acids with the C-terminal 148 amino acids of PVM M2-1 through the MSc I site. Likewise, PR M2-1 was constructed by replacing the PVM. M2-1 MSc I to BamH I restriction fragment with that of RSV. Introduction of mutations into M2-1 proteins of RSV or PVM M2-1 was performed by the Quick Change Site-directed Mutagenesis Kit (Stratagene).

pRSVlacZ minigenome encodes the β-galactosidase gene at the negative sense under the control of the T7 promoter. The lacZ gene was flanked by the RSV leader and trailer sequences as described by Tang et al., (2001) J. Virol. 75:11328-11335.

### Transfection of Cells and Expression Analysis

HEp-2 cells were infected with modified vaccinia virus (MVA) expressing T7 RNA polymerase (MVA-T7) at MOI of 1.0 and transfected with 0.4 μg of pP, 0.4 μg of pN, 0.2 μg pL, 0.4 μg pRSVLacZ together with various amounts (e.g., 0.1 μg) of M2-1 expression plasmid. Transfection was performed using lipofectACE or Lipofectamine 2000 (Invitrogen) according to the manufacturer's protocol. The transfected cells were incubated at 35° C. for 2 days and cell extracts prepared by incubating in cell permeablization buffer that contained 0.5% NP-40 and 20 mM β-Mercaptoethanol. Cell lysates were clarified by centrifugation at 2,500 rpm for 5 minutes at 4° C. and analyzed for β-galactosidase activity using 5 mM chlorophenol red-β-D-galactopyranoside (CPRG, Roche Molecular Biochemicals) as described by Tang et al. (2001) J. Virol. 75:11328-11335 and herein. The change in optical density at wavelength 550 nm (OD550) was measured with SPECTRAmax, 340PC microplate spectrophotometer using SOFTmax software (Molecular Devices). The assay was shown to be linearly responsive up to an OD₅₅₀ of 3.0. The relative activity of each mutant was calculated compared to RSV M2-1 and the data obtained was an average of a minimum of three experiments.

Synthesis of lacZ reporter RNA in transfected cells was analyzed by Northern blotting. Two days after transfection, total intracellular RNA was extracted by RNeasy extraction kit (Qiagene) and electrophoresed on 1% agarose/urea gel. The RNA blot was hybridized with Dig-labeled negative sense lacZ or M2-1 probe. The hybridized RNA was detected using Dig-RNA detection kit (Roche Biochemicals) following exposure to the X-ray film (Kodak).

### Protein Labeling and Immunoprecipitation

Phosphorylation of M2-1 and M2-1-N protein interaction in transfected cells were examined by immunoprecipitation. HEp-cells were infected with MVA-T7 and transfected with pN, pP, pL, pM2-1 and pRSVlacZ minigenome. The transfected cells were incubated at 37° C. for 18 hr and radio-labeled with ³⁵S-promix (100 μCi/ml) in DME deficient in methionine and cysteine or with ³³P-phosphate (100 μCi/ml) in DME deficient in phosphate for four hours. The cells were lysed in RIPA buffer containing 0.15 M NaCl and immunoprecipitated with anti-M2-2 monoclonal antibodies (a gift of Dr. P. Yeo) or anti-RSV polyclonal antibody (Biogenesis). After incubation with protein G agarose beads (Invitrogen) for 30 min, the immunoprecipitated complex were washed three times with RIPA buffer containing 0.3 M NaCl and electrophoresed on 4-15% gradient polyacrylamide gel (Novagen). The immunoprecipitated proteins were visualized by autoradiography (Kodak).

### Results

### Comparison of RSV and PVM M2-1 Function

PVM M2-1 (SEQ ID NO:20) is 40% identical to RSV M2-1 (SEQ ID NO:19) and contains a Cys₃-His₁ motif at its N-terminus (FIG. 2A); it is expected that this protein functions as a transcriptional processivity factor for PVM transcription. The RSV M2-1 processivity function can be measured using the RSVlacZ minigenome assay (Tang et al. (2001) J. Virol. 75:11328-11335). To examine whether PVM M2-1 protein could function in the RSV minigenome assay, MVA-T7-infected HEp-2 cells were transfected with plasmids encoding the RSV N, P and L proteins (0.2 μg of pN, 0.2 μg of pP, 0.1 μg of pL), pRSVlacZ (0.2 μg of pRSVLacZ) and various amounts of either PVM M2-1 or RSV M2-1. Two days after transaction, the level of β-galactosidase activity was determined. As shown in FIG. 2B, which shows β-galactosidase activity (OD550) versus amount of RSV (triangles) or PVM (diamonds) M2-1 plasmid transfected, the processivity function of RSV M2-1 was required for lacZ reporter expression. β-galactosidase was not detected in the absence of M2-1 but was produced in a dose-dependent manner with an increased amount of RSV M2-1 plasmid. In contrast, PVM M2-1 showed a very low processivity in this assay. A level of 2-5% of that of RSV M2-1 was reproducibly detected at a concentration of 10-50 ng of PVM M2-1 plasmid. Therefore, in contrast to RSV M2-1, PVM M2-1 exhibited a very low level of processivity in the RSVlacZ minigenome assay.

### Processivity of RSV and PVM M2-1 Chimeric Proteins

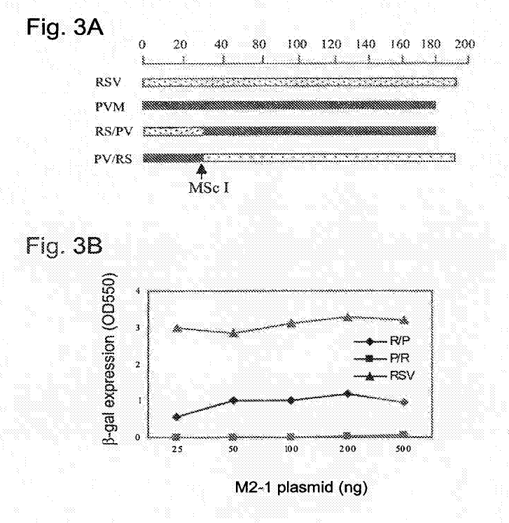

To dissect the essential functional domain of RSV M2-1 required for its processivity function, two chimeric protein expression plasmids derived from portions of the PVM and RSV M2-1 ORFs were constructed (FIG. 3A). The N-terminal 30 amino acids of the RP M2-1 chimera were derived from RSV M2-1 and the remaining C-terminal sequence derived from PVM. PR M2-1 represented the converse chimera in that its N-terminal 29 amino acids were derived from PVM and the C-terminus from RSV. The one amino acid difference in the N-terminal portions of the chimeras was accounted for by a lack of Asn-5 in the PVM sequence. These two chimeric proteins exhibited strikingly different activity in the RSVlacZ minigenome assay (FIG. 3B). PR M2-1, (squares) had an activity similar to PVM M2-1, at a level less than 5% of RSV M2-1 (triangles). However, RP M2-1 (diamonds) maintained approximately 36% of M2-1 activity. Therefore, the N-terminal region of M2-1 played an important role in determining the protein's function.

### Identification of Residues that are Critical to the M2-1 Function

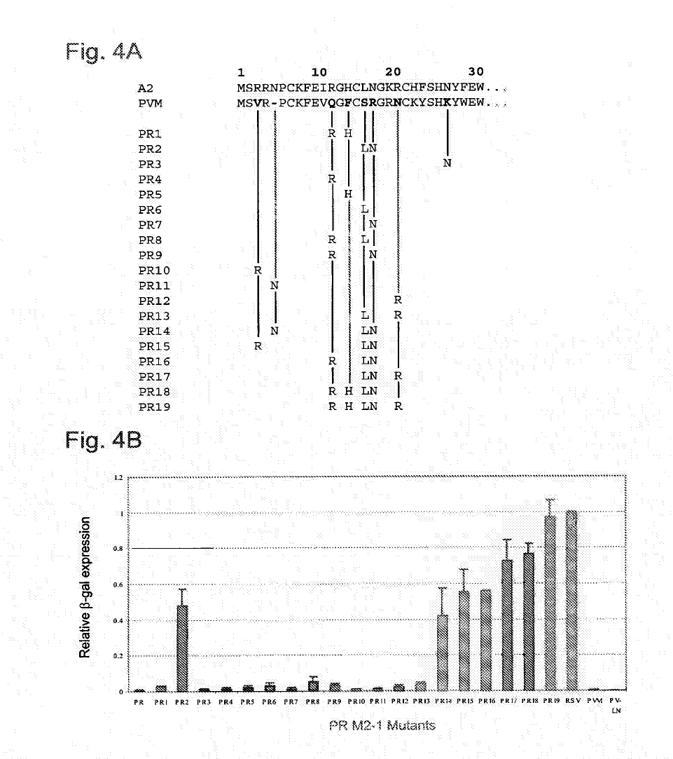

In order to identify critical residues in the N-terminal region, a systematic analysis was performed to introduce RSV sequences into PR M2-1 that restored processivity function. The 29 amino acids of the N-terminal PVM M2-1 differ from RSV by 13 amino acids and lack the Asn residue corresponding to the fifth amino acid of RSV M2-1 (FIG. 3A). Among the 13 amino acids different between RSV and PVM, five residues (I11V, K19R, H22K, F23Y and F29W) have similar biochemical properties and were not selected for substitution mutagenesis. The remaining eight amino acids in the N-terminal 30 residues of PR M2-1 were mutagenized individually or in combination to change the PVM residues to those of RSV. A total of 19 mutants were constructed (FIG. 4A) and their processivity functions were analyzed by the RSVlacZ minigenome assay (FIG. 4B). Expression of the M2-1 protein was monitored by immunoprecipitation to ensure that an equivalent level of M2-1 protein was expressed. From the 13 single and double mutations initially constructed (PR1-PR13), only those mutants that contained substitutions of both S15L and R16N had a significant increase in their processivity. Individual substitution of either S15L (PR6) or R16N (PR7) had very little positive effect on the PR M2-1 function. However, PR2- M2-1 containing only these two changes together had an increase in protein processivity to levels approximately 48% of RSV M2-1. Therefore, it was considered that other residues differed between RSV M2-1 and PVM M2-1 might also influence the protein function.

Substitutions of the PVM residues at positions 3, 11, 13, 19 and 25 by those of RSV M2-1 or insertion of Asn-5 did not increase PR M2-1 protein function. Thus, more mutagenesis was performed in PR2 M2-1 (containing S15L and R16N) to determine whether other amino acids changes could further increase its activity approaching the level of RSV M2-1 (FIG. 4A). An increase in protein function was observed for mutations that involved charged residues. Introduction of V3R (PR15) and Q11R (PR16) increased PR2 M2-1 processivity by approximately 7% and N19R (PR17) mutation increased PR2 M2-1 activity to 25% or more. Double Q11R and F13H mutations (PR18) increased PR2 M2-1 function by 27% and the triple mutations (Q11R, F13H and N19R) introduced into PR2 M2-1 resulted in a protein that had an activity almost identical to RSV M2-1. Thus, in addition to S15L and R16N residues that are critical to PR M2-1 function, several charged residues in addition are also required to produce a fully functional protein.

In order to determine whether PVM M2-1 protein processivity could be increased by introduction of Leu and Asn, mutagenesis was performed in PVM M2-1 to substitute Ser-15 and Arg-16 by Leu and Asn. Unexpectedly, PVM M2-1 bearing S15L and R16N changes (FIG. 4, PV-LN) did not have increased processivity function in the RSV minigenome assay. Thus, the C-terminal region of PVM M2-1 may have a greater influence on its function.

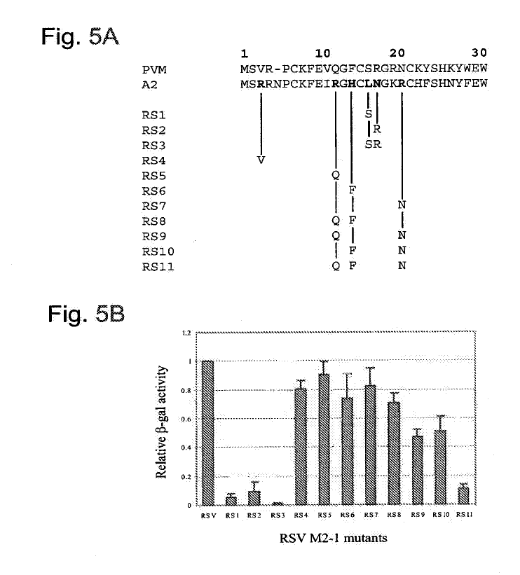

To confirm the role of Leu-16, Asn-17 and the charged residues at the N-terminus of RSV M2-1 to its function, mutagenesis was further performed in the RSV M2-1 molecule and a total of 11 mutants were generated (FIG. 5A). Single substitution mutations, L16S (RS1) or N17R (RS2), greatly reduced RSV M2-1 protein function by 97% and 94%, respectively (FIG. 5B). The double substitution mutation, L16S and N17R (RS3) further reduced the protein function and only 1% of normal lacZ activity could be detected for RS3 in the RSVlacZ minigenome assay (FIG. 5B). These data demonstrated that Leu-16 and Asn-17 are the two residues critical to the RSV M2-1 function. Substitution of single charged residues at positions of 3 (RS4), 12 (RS5), 14 (RS6) or 20 (RS7) each resulted in reduced M2-1 protein activity by 10-25%; however, none were as critical as Leu-16 and Asn-17. Substitutions of multiple charged residues had a greater effect on the RSV M2-1 function. Double substitution mutations reduced protein function by 30% for RS8, 53% for RS9 and 50% for RS10. The triple mutations bearing R12Q, H14F and R20N reduced the M2-1 function, by approximately 90%. Thus, consistent with the mutagenesis analysis of PR M2-1, the charged residues in the N-terminus of RSV M2-1 protein were important to the protein processivity function in addition to the Leu-16 and Asn-17 residues.

### M2-1 Phosphorylation and Processivity

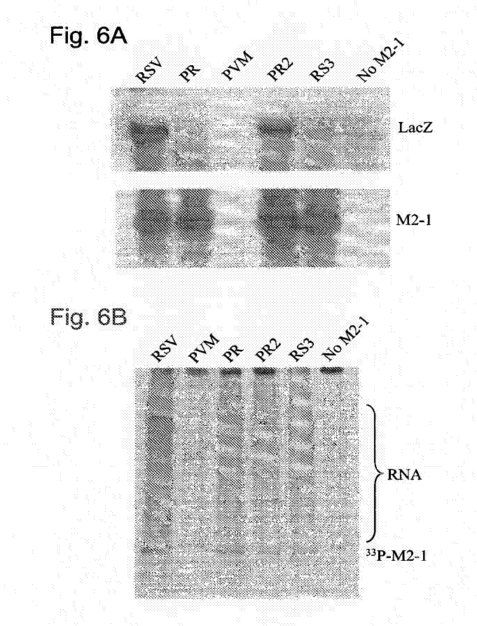

To examine whether M2-1 mutations affected M2-1 protein phosphorylation status, MVA-T7 infected HEp-2 cells were transfected with plasmids encoding the N, P, and L proteins and pRSVLacZ together with RSV, PVM, PR, PR2 or RS3 M2-1 expression plasmids in duplicate. At 24 hr post-transfection, RNA was extracted from one set of cells and a Northern blot was probed with a riboprobe specific for LacZ or M2-1 (FIG. 6A). Another set of cells was radio-labeled with ³³P-phosphate and immunoprecipitated with anti-M2-1 monoclonal antibodies (FIG. 6B). Consistent with the β-galactosidase assay, lacZ mRNA was not detected in cells expressing PVM, PR, or RS3 M2-1 or in cells that had no M2-1 protein expressed (FIG. 6A). LacZ mRNA was detected in cells expressing PR2 M2-1 at a level approximately 50% of RSV M2-1, which was also consistent to the level of β-galactosidase detected (FIG. 4B). Except for PVM M2-1 that was not detected by RSV M2-1 probe due to low sequence homology, a comparable level of M2-1 mRNA was produced in the cells transfected with all the mutants. Again, except for PVM M2-1 that was not detected by anti-RSV M2-1 antibodies, PR, PR2, and RS3 M2-1 proteins were phosphorylated regardless of their processivity activity. Each of the proteins was also able to bind to RNA as shown by the presence of ³³P-labeled co-immunoprecipitated materials that was sensitive to RNase A treatment (data not shown, Cartee & Wertz. 2001, J Virol 75:12188-12197) and was low or absent in the cells that expressed PVM M2-1 or had no M2-1 protein expressed. These data suggested that chimeric PR M2-1 or M2-1 mutations did not result in significant changes in mRNA synthesis, protein phosphorylation or RNA-binding ability of M2-1 mutants.

### Effect of M2-1 Mutations on M2-1 and N Interaction

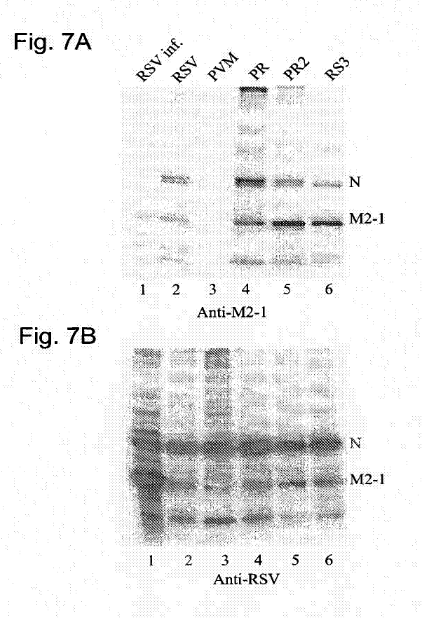

To determine if the difference in M2-1 processivity was due to any alternations of M2-1 and N protein interaction, HEp-2 cells were transfected with N, P and L expression plasmids, pRSVLacZ and M2-1 expression plasmids, radio-labeled with ³⁵S-Met/³⁵S-Cys and immunoprecipitated 18 h post-transfection with monoclonal antibodies against RSV M2-1 (FIG. 7A) or a polyclonal antibody against RSV (FIG. 7B). RSV infected cells produced less N protein than the transfected cells in this experiment (FIG. 7B, lane 1) and the N protein immunoprecipitated by anti-M2-1 antibodies was detected in a longer exposure. A comparable level of N and M2-1 proteins was detected in each transfected cells as shown by immunoprecipitation using anti-RSV antibody (FIG. 7B). Except for PVM M2-1 that was not recognized by anti-M2-1 antibodies, the N proteins were co-immunoprecipitation with the M2-1 protein of RSV, PR, PR2 or RS3 (FIG. 7A). The slower migrating M2-1 represented the phosphorylated form, which is more abundant in the transfected cells (lanes 2-6) than in the RSV-infected cells (lane 1). Although the level of the N proteins coprecipitated by each M2-1 protein varied between experiments, it did not appear to have direct correlation with the M2-1 protein function.

### Example 2

### Mutations in RSV P Protein that Confer Temperature Sensitivity

### Materials and Methods

### P Gene Library Construction and Screening

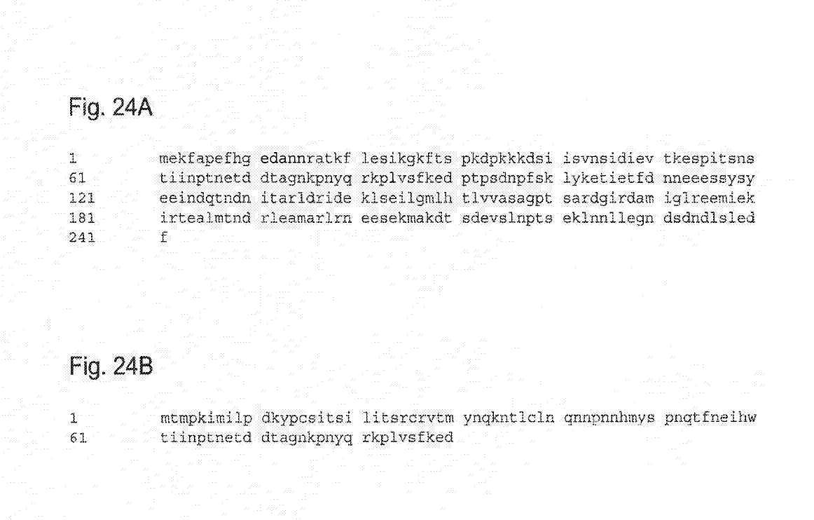

A P gene cDNA mutant library was constructed by random mutagenesis of the C-terminal 96 codons of the P gene. Mutagenesis was accomplished by low fidelity PCR amplification with exonuclease-deficient PFU DNA polymerase (Stratagene) and primers 5′AvrII (5′-GATAATCCCTTTTCTAAACTATAC; SEQ ID NO:3) and 3′Act2(5′-CATTTAAAAAATTCTATAGATCAGAGG; SEQ ID NO:4) using pGAD GL-P as the template. The 5′AvrII primer annealed to sequences approximately 150 bp upstream of the silent AvrII site in the P ORF, and the 3′Act2 primer annealed to sequences approximately 150 bp downstream of the Xhol site in the pGDL GL vector. The randomly introduced mutations in the PCR cDNA fragments were then transformed into the yeast Saccharomyces cerevisiae Y190 strain, together with pAS2-N and the gapped pGAD GL-P that had the C terminus of the P gene removed by digestion with AvrII and Xhol restriction enzymes. Recombination of the gapped vector with the random PCR fragments generated aP gene cDNA library. To identify temperature sensitive (ts) P mutants, the transformants were replica plated on two SD-Leu-Trp plates (Bio 101) without additives; two SD-Leu-Trp-His plates containing 50 mM 3 aminotriazole (3-AT); one SD-Leu-Trp-His plate containing 100 mM 3-AT; and one SD-Leu-Trp-His plate containing 150 mM 3-AT, The duplicate plates were incubated at 30 and 37° C., respectively, and the single plates were incubated at 30° C. for 3 days. Colonies that showed no growth or highly reduced growth on the SD-Leu-Trp-His plates containing 50 mM 3-AT at 37° C. but still showed good growth at least on the SD-Leu-Trp-His plates containing 100 mM 3-AT at 30° C. were picked. A total of 64 ts mutants were identified. The pGAD GL-P mutant plasmids were isolated from the yeast cells, amplified in Escherichia coli and retransformed into the Y190 strain along with pAS2-N to confirm the temperature-dependent N-P interaction on the replica plates as described above. The P gene mutants that exhibited ts interaction were sequenced to identify the mutations. The sequence of P protein of the wild-type human RSV A2 strain is provided in FIG. 24.

### Cells, Viruses, and Antibodies

Monolayer cultures of HEp-2 and Vero cells (obtained from the American Type Culture Collections [ATCC]) were maintained in minimal essential medium containing 5% fetal bovine serum (FBS). Recombinant RSV A2 (rA2) was recovered from an antigenomic cDNA derived from RSV A2 strain, pRSVC4G (Jin et al. (1998) Virology 251:206-214), and grown in Vero cells. The modified vaccinia virus Ankara strain expressing bacteriophage T7 RNA polymerase, MVA-T7 (Wyatt et al. (1995) Virology 210:202-205), was provided by Bernard Moss and grown in CEK cells. Polyclonal anti-RSVA2 antibodies were obtained from Biogenesis (Sandown, N.H.), Monoclonal anti-RSV P antibodies 1P, 02/021P, and 76P were provided by Jose A. Melero.

### Screening N-P Protein Interaction in the Yeast Two-Hybrid System

The interaction of the RSV N and P proteins was established by using the yeast two-hybrid system (Clontech). The two hybrid fusion plasmids were constructed as follows. The N open reading frame (ORF) of RSV was fused in frame with the GAL4 DNA-binding domain in the vector pAS2 through NcoI and EcoRI restriction sites. The P ORF was fused in frame with GAL4 activation domain in the pGAD GL vector through the BamHI and Xhol restriction sites. A silent AvrII site was introduced at codon 145 of the P ORF in pGAD GL-P to facilitate the construction of the P cDNA gene library. The mutagenesis was performed with a QuikChange mutagenesis kit (Stratagene) with a pair of primers, 5′-GAAAAATTAAGTGAAATCCTAGGAATGCTTCAC: SEQ ID NO:5 (the AvrII site is underlined) and its complementary sequence.

### Functional Analysis of P Mutants by RSV Minigenome Replication Assay

Plasmids expressing RSV N, P, and L under the control of the T7 promoter were described previously (Jin et al. (1998) Virology 251:206-214). The P gene was mutated using either the QuikChange site-directed mutagenesis kit or the ExSite PCR-based site-directed mutagenesis kit (Stratagene). The following changes were made in the pP plasmid: G172S, E176G, G172S/E176G; 174-176A (R174A/E175A/E176A), ΔC6 (deletion of six amino acids from the C terminus) and Δ61-180 (deletion of residues from 161 to 180), RSV replication was assayed by using a RSV minigenome replicon, pRSV-CAT (Tang et al. (2001) J. Virol. 75; 11328-11335). For minigenome assays, HEp-2 cells in 12-well plates were infected with MVA-T7 at a multiplicity of infection (MOI) of 5 PFU/cell and then transfected with 0.2 μg of pRSV-CAT, together with 0.2 μg of pN, 0.1 μg of pL, and 0.2 μg of wild-type (wt) pP or mutant pP in triplicate. The transfected cells were incubated for 48 h at 33, 37, or 39° C. The amount of chloramphenicol acetyltransferase (CAT) protein expressed in the transfected cells was determined by an enzyme-linked immunosorbent assay (Roche Molecular Biochemicals). The protein expression levels of N and P in the transferred cells were determined by Western blotting. Total cellular polypeptides were electrophoresed by sodium dodecyl sulfate-polyacrylamide gel electrophoresis (SDS-PAGE) on 15% acrylamide gels and transferred onto nylon membranes (Amersham Pharmacia Biotech). The blots were incubated with goat anti-RSV antibody (Biogenesis) and subsequently with a horseradish peroxidase-conjugated rabbit anti-goat immunoglobulin G (Dako). The membrane was incubated with the enhanced chemiluminescence substrate (Amersham Pharmacia Biotech). Protein bands were visualized after exposure to BioMAX ML film (Kodak).

### Recovery of Recombinant RSV

The G172S and E176G mutations were introduced individually into the RSV antigenomic cDNA clone. E176G mutations contained two nucleotide changes from GAA to GGT. Mutations were first introduced into a RSV cDNA subclone, pRSV-(A/S), which contains the RSV A2 sequences from nucleotide 2128 (AvrII) to nucleotide 4485 (SacI), by using the QuikChange site-directed mutagenesis kit (Stratagene). The AvrII-SacI fragment carrying the introduced mutations was then shuttled into the full-length RSV A2 antigenomic cDNA clone, pRSVC4G (Jin et al. (1998) Virology 251:206-214). pRSVC4G contains the C-to-G change at the fourth position of the leader region in the antigenomic sense (Jin et al. (1998) Virology: 251:206-214). Recombinant viruses were recovered from the transfected HEp-2 cells as described previously (Jin et al. (1998) Virology 251:206-214) and designated rA2-P172 and rA2-P176. The recovered viruses were plaque purified and amplified in Vero cells. The virus titer was determined by plaque assay on Vero cells, and the plaques were enumerated after immunostaining them with a polyclonal anti-RSV A2 serum (Biogenesis). The presence of each mutation in the rescued viruses was confirmed by sequence analysis of the P gene cDNA amplified by reverse transcription-PCR by using the viral genomic RNA as a template.

### Replication of rA2-P172 and rA2-P176 in HEp-2 and Vero Cells

Plaque formation of each mutant was examined in HEp-2 and Vero cells at 33, 37, 38, and 39° C. Cell monolayers in six-well plates were infected with 10-fold serially diluted virus and incubated under an overlay that consisted of L15 medium containing 2% FBS and 1 % methylcellulose in a submerged water bath for 6 days. The plaques were visualized and enumerated after immunostaining with a polyclonal antiserum against RSV A2 (Biogenesis). The plaques were photographed under an inverted microscope for plaque sizes comparisons.

The growth kinetics of rA2-P172, rA2-P176 in comparison with wt rA2 was studied in both HEp-2 and Vero cells. Cells in six-well plates were infected with wt rA2, rA2-P172, or rA2-P176 at an MOI of 1.0 or 0.01 PFU/cell. After 1 h of adsorption at room temperature, the infected cells were washed three times with phosphate-buffered saline, overlaid with 3 ml of Opti-MEM I (Life Technologies), and incubated at either 33 or 38° C. At 24-h intervals, 200 μl of culture supernatant was collected and stored at −80° C. in the presence of SPG prior to virus titration (Tang et al. (2001) J. Virol. 75:11328-11335). Each aliquot taken was replaced with an equal amount of fresh medium. The virus titer was determined by plaque assay on Vero cells at 33° C.

### Coimmunoprecipitation of the N and P Proteins

Coimmunoprecipitation was performed to study the interaction between the N and P proteins. For transient protein expression, MVA-T7-infected HEp-2 cells in 12-well plates were cotransfected with 2 μg each of pN and pP plasmid by using LipofectACE (Life Technologies). To examine the N-P interaction in virus-infected cells, Vero cells were infected with rA2, rA2-P172, or rA2-P176 at an MOI of 1.0 PFU/cell. The transfected or recombinant RSV-infected cells were incubated at 33, 37, or 39° C. for 12 h and then exposed to [³⁵S]Cys and [³⁵S]Met (100 μCi/ml) in Dulbecco modified Eagle medium (DMEM) deficient in cysteine and methionine for 4 h. The radiolabeled cell monolayers were lysed in the radioimmunoprecipitation assay buffer (10 mM Tris-HCl, pH 7.5; 150 mM NaCl; 5 mM EDTA; 1% Triton X-100; 1% sodium deoxycholate; 0.1% SDS). The polypeptides were immunoprecipitated with polyclonal goat anti-RSV A2 antibodies or with a mixture of monoclonal antibodies (1P, 021P, and 76P) against the P protein at 4° C. for 12 h. The antibody-protein complex was precipitated by the addition of 30 μl of protein G-agarose beads (Life Technologies) at 4° C. for 30 min and washed three times with radioimmunoprecipitation assay buffer containing 300 mM NaCl. The immunoprecipitated polypeptides were electrophoresed by SDS-15% PAGE and detected by autoradiography. The N and P proteins detected on the autoradiographs were quantified by densitometry with a Molecular Dynamics densitometer by using ImageQuant 5.0 for Windows NT (Molecular Dynamics).

### Virus Replication in Mice and Cotton Rats

Virus replication in vivo was determined in respiratory pathogen-free BALB/c mice and cotton rats (Sigmodon hispidus [Harlan]). Mice or cotton rats in groups of eight were inoculated intranasally under light methoxyflurane anesthesia with 0.1 ml of inoculum containing 10⁶ PFU of virus per animal. At 4 days postinoculation, the animals were sacrificed by CO₂ asphyxiation, and their lungs were harvested. The tissues were homogenized in Opti-MEM I (Life Technologies), and the virus titer was determined by plaque assay on Vero cells.

### Results

### Identification of P Mutations that Weaken the N-P Interaction in the Yeast Two-Hybrid Assay

To identify mutations in the P protein that destabilize its interaction with the N protein, a yeast two-hybrid assay was used to screen a randomly mutagenized P cDNA library with mutations introduced in the C-terminal 96 codons of P for mutants that permitted interaction of P with N at the permissive temperature of 30° C. but prevented interaction with N at the nonpermissive temperature of 37° C. The wt N and P proteins interacted with each other in yeast as indicated by the growth of the cotransformed yeast strain on the selective medium at 30° C. as well as at 37° C. The transformants were screened for mutants that were capable of activating the yeast two-hybrid reporter gene at the permissive temperature of 30° C. but not at 37° C. From approximately 1,300 original transformants, 64 possible ts mutants were identified. These putative ts clones were subjected to a second round of screening in yeast Y190. Two transformants were confirmed for their is phenotypes. The pGAD GL-P plasmids from these yeast clones were sequenced. The mutations were identified as either Gly at residue 172 replaced by Ser (G172S) or Glu at residue 176 replaced by Gly (E176G) as shown in FIG. 1.

### Immunoprecipation Analysis of N-P Interaction in Cells Transiently Expressing N and P

Sequence alignment of the P proteins of several pneumoviruses revealed that residues 172 and 176 and the adjacent regions are highly conserved and contain several charged residues (FIG. 1). To examine the functional role of the charged residues, a mutant P protein expression plasmid was constructed in which each of the three charged residues, REE at positions 174 to 176, was replaced with alanine. In addition, a plasmid containing both G172S and E176G mutations in the P gene was constructed. Two deletion mutants either lacking the C-terminal six residues or lacking residues from 161 to 180, which both have been shown to interfere with N-P interactions in RSV (Garcia-Barreno et al. (1996) J. Virol. 70:801-808; Khattar et al. (2001) J. Gen. Virol. 82:775-779), were made.

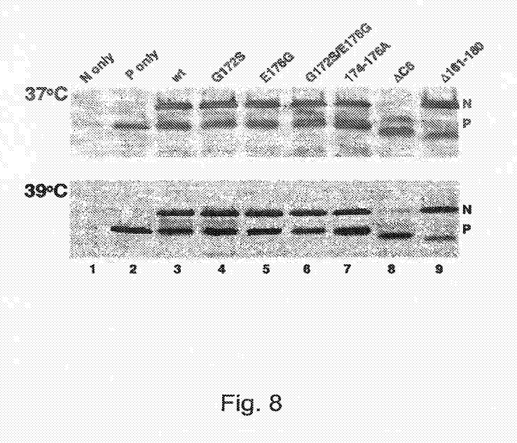

To examine the effects of the P mutations on the N-P interaction, MVA-T7-infected HEp-2 cells were cotransfected with pN and pP mutant plasmids and incubated at 37 or 39° C. The ³⁵S-labeled polypeptides were immunoprecipitated by anti-P monoclonal antibodies. As shown in FIG. 8, the N protein was only precipitated by anti-P antibodies in the presence of the P protein (lane 1 versus lane 3), demonstrating that the immunoprecipitation of the N protein occurred through its interaction with the P protein. Deletion of the six residues from the C terminus of the P protein drastically reduced its interaction with the N protein; only a trace amount of N was detected (FIG. 8, lane 8). The N protein was coprecipitated by all of the other P mutants, G172S, E176G, G172S/E176G, 174-176A, and 161-180.The amount of 161-180 P protein detected on the gel was less than that of wt P, possibly because of the removal of the two potential ³⁵S-labeled methionines in this region. Thus, coimmunoprecipitation of N and P in transiently expressed cells did not reveal any defect in N-P interaction for G172S and E176G mutations.

FIG. 8 illustrates an immunoprecipitation analysis of N-P interaction in cells transiently expressing N and P. MVA-T7-infected HEp-2 cells were transfected with pN and different pP protein expression plasmids under the control of T7 promoters and incubated for 16 h at 37° C. (upper panel) or 39° C. (lower panel). The proteins were radiolabeled with [³⁵S]Cys and [³⁵]Met (100 μCi/ml) in DMEM deficient in cysteine and methionine for 4 h, immunoprecipitated by anti-P monoclonal antibodies, separated on a 15% polyacrylamide gel, and exposed to Kodak BioMAX film. The positions of N and P are indicated on the right.

### Effects of P Mutations on the Replication and Transcription of the RSV-CAT Minigenome

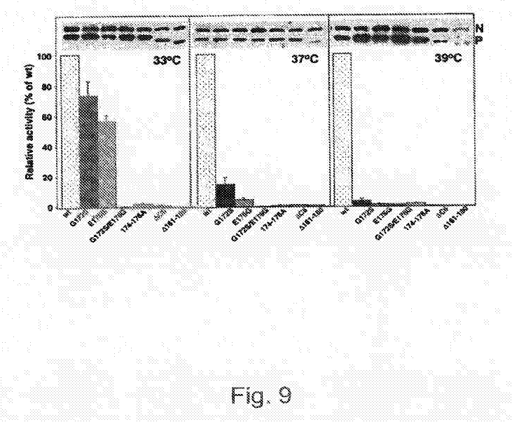

The function of the P mutants was analyzed by a CAT minigenome replication assay. The mutant P expression plasmids were transfected, together with pN, pL, and pRSV-CAT, into MVA-T7 infected HEp-2 cells, and CAT expression was measured at 33,17, or 39° C. The levels of N and P protein expression were determined by Western blotting with polyclonal anti-RSV antibodies (insets, FIG. 9). CAT reporter gene activities produced by different P mutants were determined by CAT-enzyme-linked immunosorbent assay and are expressed as the percentage of that of wt P at each temperature. The error bars show the standard deviations of three replicate experiments.

As shown in FIG. 9, at 33° C., CAT protein expression was detected in cells expressing the mutant P proteins containing either G172S or E176G, although their activities were reduced by ca. 24 and 45%, respectively. At 37° C., the level of the CAT protein detected was reduced by ca. 80% for G172S and 90% for E176G. The reduction was even greater (>95%) at 39° C. These data indicated that mutations displayed a conditional ts phenotype consistent with the ts interaction phenotype observed in the yeast two-hybrid assays. No CAT expression was detected in cells expressing the P protein containing the combined G172S and E170G mutations, substitution of the three charged residues at positions 174 to 176 by alanine, a deletion of six amino acids from the C-terminal, end or an internal 20 amino acid deletion.

To eliminate the possibility that the reduction in reporter gene expression was caused by altered protein expression of these P mutants at higher temperatures, the levels of the P and N proteins produced in the transfected cells were examined by Western blotting. Except for 161-1:80 mutant, all of the other P mutants expressed a comparable level of protein (FIG. 9, insert). Therefore, the reduced ability of the other mutants to support RSV minigenome replication was a direct result of the introduced mutations rather than changes in their protein levels in the transfected cells.

### Replication of rA2-P172 and rA2-P176 in Cell Cultures

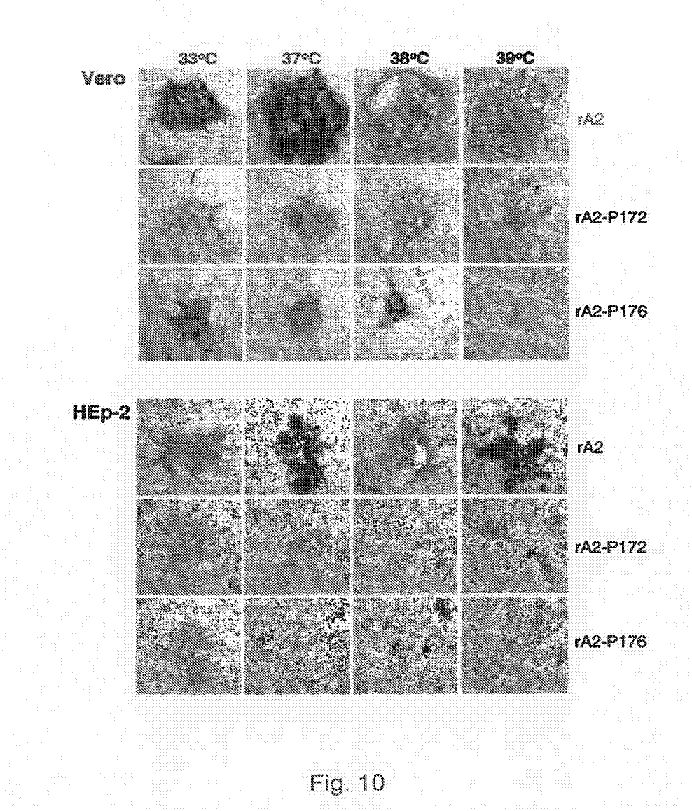

The G172S and E176G mutations were individually introduced into the full-length RSV antigenomic cDNA clone, and recombinant viruses were generated. Both rA2-P172 and rA2-P176 reached peak titers of ca. 2×10⁷ PFU/ml in Vero cells at 33° C., a level comparable to that of wt rA2. The plaque formation efficiency of rA2-P172 and rA2-P176 at different temperatures was examined in Vero and HEp-2 cells and is summarized in Table 3 and FIG. 10.

Monolayers of Vero cells (FIG. 10, upper panel) and HEp-2 cells (FIG. 10, lower panel) were infected with wt rA2, rA2-P172 and rA2-P176; overlaid with L15 medium containing 1% methylcellulose and 2% FBS; and incubated at 33, 37, 38, and 39° C. for 6 days. The plaques were visualized by immunostaining with polyclonal anti-RSV antibodies. Plaques were photographed on a Nikon inverted microscope. Arrows in the lower panels indicate RSV-infected HEp-2 cells at 38 and 39° C.

Both rA2-P172 and rA2-P176 formed smaller plaques than wt rA2 at 37° C. and higher temperatures. No plaques were visualized for rA2-P172 in Vero cells and HEp-2 cells at 39° C., although RSV-infected single or multiple cells stained by anti-RSV antibody were observed under the microscope. Likewise, no visible plaques were observed for rA2-P176 in Vero cells or HEp-2 cells at 39° C. and in HEp-2 cells at 38° C. rA2-P176 was more temperature sensitive than rA2-P172: the shutoff temperature for rA2-P172 was 39° C. in HEp-2 and Vero cells whereas the shutoff temperatures for rA2-P176 were 38° C. in HEp-2 cells and 39° C. in Vero cells (Table 3).

TABLE 3
Efficiency of plaque formation of RSV P mutants at various
temperatures
Mean virus titer (log 10 PFU/ml) in Vero or HEp-2 cells^{a}

| | 33° C. | | 37° C. | | 38° C. | | 39° C. | |
|---|---|---|---|---|---|---|---|---|
| Virus | Vero | HEp-2 | Vero | HEp-2 | Vero | HEp-2 | Vero | HEp-2 |
| rA2 | 6.70 | 6.61 | 6.73 | 6.52 | 6.69 | 6.48 | 6.63 | 6.46 |
| rA2- | 6.59 | 6.41 | 6.54* | 6.33* | 6.51* | 5.93* | —^{b} | — |
| P172 | | | | | | | | |
| rA2- | 6.65 | 6.53 | 6.64* | 6.24* | 5.54* | — | — | — |
| P176 | | | | | | | | |

^{a}Virus Titers are the average of two independent experiments from two different virus stocks.
^{b}indicates no visible plaques
*small plaque size

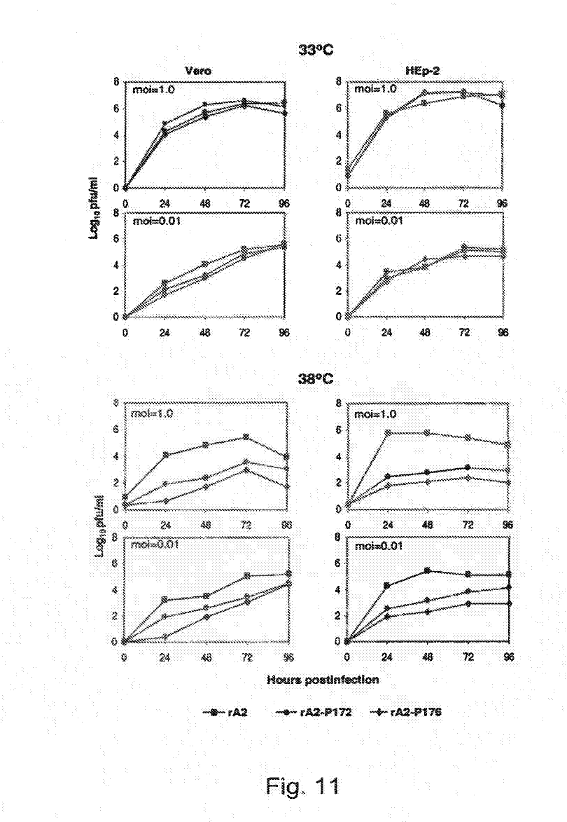

The single-cycle (MOI=1.0) and multicycle (MOI=0.01) growth kinetics of rA2-P172 (circles) and rA2-P176 (diamonds) were compared to those of rA2 (squares) in both HEp-2 and Vero cells (FIG. 11). Vero or HEp-2 cells were infected with virus at an MOI of 1.0 or 0.01 PFU/cell and incubated at 33 or 38° C. Aliquots of culture supernatants (200μl) were harvested at 24-h intervals for 5 days, and the virus titers were determined by plaque assay on Vero cells. Each virus titer is an average of two experiments. At 33° C., both rA2-P172 and rA2-P176 had similar replication kinetics and reached peak titers comparable to that of rA2 at both MOIs in both cell lines. At 38° C., rA2-P172 and rA2-P.176 reached peak titers much lower than that of wt rA2. At an MOI of 1.0 PFU/cell, rA2-P172 had peak titers ca. 2.0 and 2.3 log₁₀ lower than those of rA2 in Vero cells and HEp-2 cells at 38° C., respectively. The reductions of rA2-P176 in its peak titer relative to wt rA2 at 38° C. were even, greater: 2.5 and 3.0 log₁₀ in Vero cells and HEp-2 cells, respectively. The reduction was less pronounced when an MOI of 0.01 was used in infection: 0.6 log₁₀ in Vero cells and 1.0 log₁₀ in HEp-2 cells for rA2-P172 and 0.8 log₁₀ in Vero cells and 2.2 log₁₀ in HEp-2 cells for rA2-P176. At 39° C., both rA2-P172 and rA2-P176 replicated to a level below the assay limit. These data are consistent with what had been observed in the minigenome assay, in which E176G was more impaired in its functions (FIG. 9).

### Replication of rA2-P172 and rA2-P176 in Mice and Cotton Rats

The replication of rA2-P172 and rA2-P176 in the lower respiratory tracts of mice and cotton rats was examined (Table 4). The replication of rA2-P172 and rA2-P176in the lungs of mice was reduced by 2.7 and 3.7 log₁₀, respectively. The replication of rA2-P172 and rA2-P176 in the lungs of cotton rats was reduced by 1.5 and 2.5 log₁₀, respectively. Consistent with the result from the minigenome assay at 37° C. and the growth kinetics in cell culture at 38° C., rA2-P176 was more attenuated than rA2-P172 as measured by replication in the lower respiratory tracts of mice and cotton rats.

TABLE 4
Replication of recombinant RSV in mice and cotton rats

Virus titer in lungs (mean
log₁₀ PFU/g ± SE)^{a} in:

| Virus | mice | cotton rats |
|---|---|---|
| rA2 | 4.64 ± 0.08 | 4.72 ± 0.08 |
| rA2-P172 | 1.97 ± 0.99 | 3.29 ± 0.39 |
| rA2-P176 | 0.90 ± 1.20 | 2021 ± 0.11 |

^{a}Groups of eight BALB/c mice or cotton rats were inoculated with 106 PFU of virus intranasally under light anesthesia on day 0 and sacrificed on day 4. Virus titers from the lung tissues were determined by plaque assay.

### Analysis of N-P Interaction in Virus-Infected Cells By Immunoprecipitation

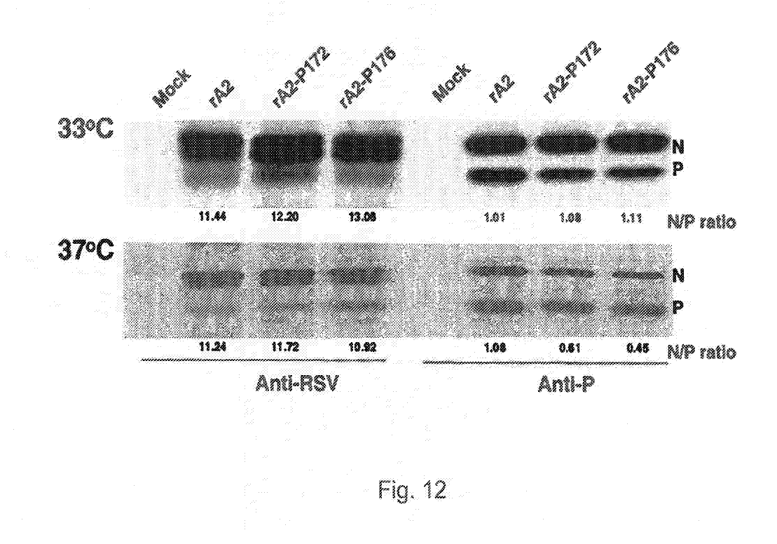

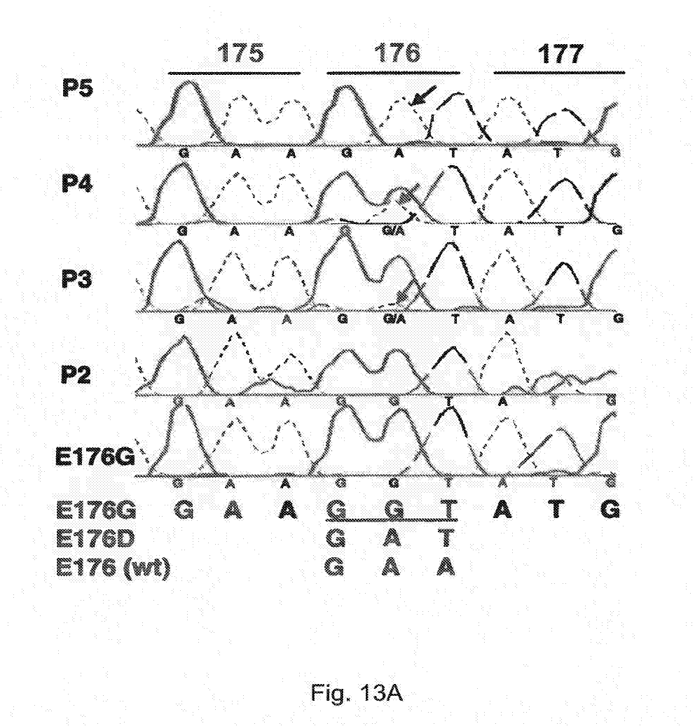

To examine whether the G172S and E176G mutations in P affected their interaction with the N protein in virus-infected cells, viral proteins from cells infected with rA2, J-A2-P172, and rA2-P176 were immunoprecipitated with either polyclonal anti-RSV antibodies or a mixture of monoclonal anti-P antibodies (FIG. 12). Anti-P monoclonal antibodies precipitated both N and P, demonstrating the formation of N-P complex in the infected cells. The H protein precipitated by anti-RSV antibody appeared as a double band but as a single band when precipitated together with the P protein by anti-P antibodies. The faster-migrating species of N may represent an unmodified form of N, which was not coimmunoprecipitated with P. There was an overall reduction in the total amount of viral proteins produced in rA2-P172- and rA2-P176-infected cells at 39° C., as expected from the observed growth kinetics in Vero cells. Therefore, coimmunoprecipitation was performed for the infected cells that were incubated at 33 and 37° C. Anti-RSV antibody did not react well with the P protein, but rA2-P172 and rA2-P176 had an N/P ratio similar to that of wt rA2 when precipitated by anti-RSV antibody at 33 and 37° C. The amounts of the N and P proteins immunoprecipitated by anti-P antibodies on the autographs were quantified by densitometry, and their relative ratios are indicated in FIG. 12. At 33° C., the N/P ratios of rA2-P172, and rA2-P176 were similar to that of rA2, indicating that the N-P interaction was not affected at the lower temperature. However, at 37° C., the amount of N coprecipitated by P was reduced in cells infected with rA2-P172 and rA2-P176. The average ratio of the N and P proteins for wt rA2 was 1.08 at 37° C. The N/P ratio of rA2-P172 was 0.61 or at a level of 56% of wt rA2; rA2-176 bad an even lower N/P ratio of 0.45 or at a level of 42% of wt rA2. The reduced N/P ratio for rA2-172 and rA2-176 at 37° C. was reproducible, demonstrating that the G172S and E176G mutations decreased the interaction between N and P at high temperatures with the E176G mutation being more impaired than G172S.

For FIG. 12, Vero cells were infected with wt rA2, rA2-P172, or rA2-P176 at an MOI of 1.0 and incubated at 33 and 37° C. for 18 h. Proteins were then radiolabeled with [³⁵S]Cys and [³⁵S]Met (100 μCi/ml) in DMEM deficient in cysteine and methionine for 4 h, immunoprecipitated by either anti-RSV or anti-P monoclonal antibodies, separated by SDS-15% PAGE, and autoradiographed. The positions of the N and P proteins are indicated on the right. The N and P ratio for each mutant was determined from four independent experiments.

### Stability of the P ts Mutations in rA2-P172 and rA2-P176

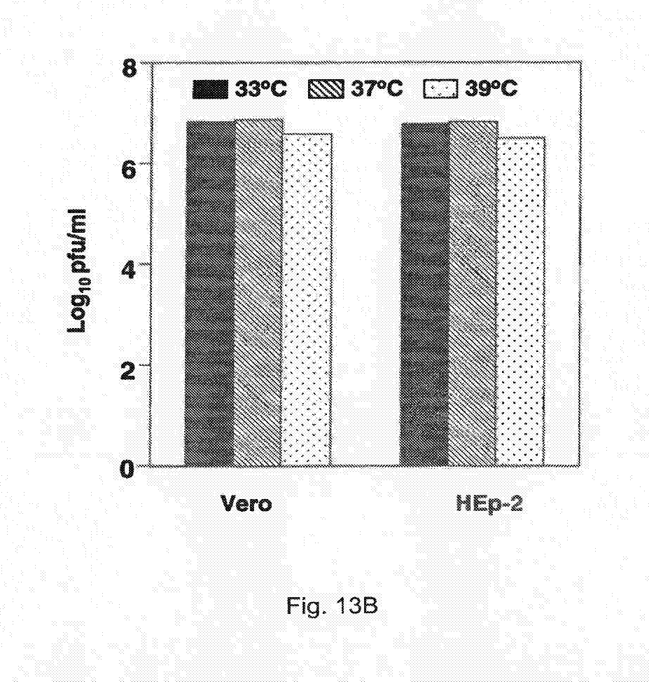

To examine the stability of the G172S and E176G mutations in the P protein, rA2-P172 and rA2-P176 were passaged in Vero cells in duplicate at 33 and 37° C. five consecutive times. Viral RNA was extracted from the infected cell culture supernatant, and the P gene cDNA was amplified by reverse transcription-PCR and sequenced. The G172S mutation was maintained at both 33 and 37° C. The E176G mutation, however, rapidly changed from Gly to Asp starting from passage 3 at 37° C. in one set of the passage samples. More than 95% of the virus population contained the E176D change at passage 5. FIG. 13A shows the sequence of the P gene in the region of residue 176 from rA2-P176 passaged in Vero cells. The introduced E176G mutation was progressively reverted to E176D starting from passage 3 (P3). Arrows indicate the G-to-A change in the 176 codon. No changes were detected at position 176 when the infected cells were incubated at 33° C.

The E176D virus was then examined for replication at various temperatures. Monolayers of Vero and HEp-2 cells were infected with rA2 P-E176D; overlaid with L15 medium containing 1% methycellulose and 2% FBS; and incubated at 33, 37, and 39° C. As shown in FIG. 13B, only a slight reduction in virus titer was observed at 39° C. compared to that seen at 33° C. Thus, virus bearing the E176D change was no longer temperature sensitive at 39° C. Sequence analysis of the second set of rA2-P176 passaged five times at 37° C. indicated mixed residues at the 176 position. Virus was then plaque purified, and the P gene cDNA was sequenced. Of eight plaque isolates, four contained Asp changes, two contained Cys, and the remaining two had Ser changes. Substitutions of Gly by Cys or Ser also resulted in the loss of the virus ts phenotype. From these results, it appeared that the negatively charged residue at position 176 was preferred by virus, with Cys or Ser as the second choice. Cys and Ser each contain side chains that can form a disulfide bond or a hydrogen bond, respectively, implying that the residue at 176 of P is involved in protein interaction.

### Example 3

### Mutation of Phosphorylation Sites in P Protein

### Materials and Methods

### Cells, Viruses, and Antibodies

Monolayer cultures of HEp-2 and Vero cells (obtained from American Type Culture Collection) were maintained in minimal essential medium (MEM) containing 5% fetal bovine serum (FBS). Recombinant RSV A2 (rA2) was recovered from an antigenomic cDNA derived from an RSV A2 strain, pRSVC4G (Jin et al. (1998) Viology 251:206-214), and grown in Vero cells. The modified vaccinia virus Ankara strain expressing bacteriophage T7 RNA polymerase, MVA-T7 (Wyatt et al. (1995) Virology 210:202-205), was provided by Bernard Moss and grown in CEK cells. Polyclonal antiRSVA2 antibodies were obtained from Biogenesis (Sundown, N.H.). Monoclonal anti-RSV P protein antibodies IP, 02/021P, and 76P were gifts from Jose A. Melero.

### Functional Analysis of P Protein Mutants by RSV Minigenome Replication Assay

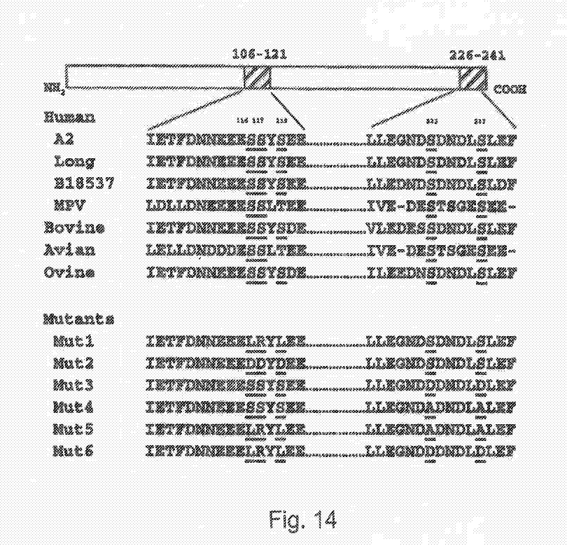

The plasmids expressing RSV N P, and L proteins under the control of the T7 promoter (in the pCITE vector) were described previously (Jin et al. (1998) Viology 251:206-214). The RSV minigenome, pRSVCAT, encodes a negative-sense chloramphenicol acetyltransferase (CAT) gene under the control of the T7 promoter (Lu et al. (2002) J. Virol. 76:2871-2880). pRSVCAT/EGFP was constructed by inserting an enhanced green fluorescent protein (EGFP) gene which was flanked by the RSV gene start and gene end sequence downstream of the CAT gene, into pRSVCAT. Phosphorylation mutations were engineered in the P protein gene by using the QuikChange Site-Directed Mutagenesis kit (Stratagene). The major phosphorylation mutations engineered in P protein are indicated in FIG. 14.

The effect of the P protein phosphorylation mutations on RSV replication was assayed with an RSV CAT minigenome system. HEp-2 cells in 12-well plates were infected with MVA-T7 at a multiplicity of infection (MOI) of 5 for 1 h followed by transfection with 0.2 μg of pRSV-CAT or pRSVCAT/EGFP together with 0.2 μg of plasmid pN, 0.1 μg of pL, and 0.2 μg of wild-type pP or mutant pP, in triplicate. The amount of CAT protein expressed in pRSVCAT and pRSVCAT/EGFP-transfected cells was determined by an enzyme-linked immunosorbent assay (ELISA) (Roche Molecular Biochemicals). The expression of the genomic RNA and CAT mRNA in the transfected cells was examined by Northern blotting with a digoxigenin (DIG)-labeled negative-sense CAT riboprobe.

### Recovery of Recombinant RSV

Two phosphorylation mutations containing two serine site substitutions (SSSAA [PP2]) or five serine site substitutions (LRLAA [PP5]) were introduced into rA2. Mutations were initially introduced into the P protein gene in an RSV cDNA subclone, pRSV-(A/S), which contains the RSV A2 sequences from nucleotide (nt 2128 (AvrII) to (nt 4485 (SacI), by the QuiKChange Site-Directed Mutagenesis kit (Stratagene). The AvrII-SacI fragment carrying the introduced mutations was then inserted into the full-length RSV A2 antigenomic cDNA clone, pRSVC4G, pRSVC4G contains the C-to-G change at the fourth position of the leader region in the antigenomic sense. Two recombinant viruses were recovered from the transfected HEp-2 cells and designated as rA2-PP2 (SSSAA) and rA2-PP5 (LRLAA). The recovered viruses were plaque purified and amplified in Vero cells. Virus titer was determined by plaque assay on Vero cells, and the plaques were enumerated after immunostaining with a polyclonal anti-RSV A2 serum (Biogenesis). The presence of each mutation in the recombinant viruses was confirmed by sequence analysis of the P protein gene cDNA amplified by reverse transcription-PCR (RT-PCR) with viral genomic RNA as template.

### Replication of rA2-PP2 and rA2-PP5 in Hep-2 and Vero cells

The plaque formation efficiency of each mutant was examined in HEp-2 and Vero cells. Cell monolayers in six-well plates were infected with 10-fold serially diluted virus and incubated under an overlay consisting of L15 medium containing 2% FBS and 1% methylcellulose for 6 days at 35° C. The plaques were visualized and enumerated after immunostaining with a polyclonal anti-RSV A2 serum.

The growth kinetics of rA2-PP2 and rA2-PP5 in comparison with those of rA2 were studied in both HEp-2 and Vero cells. Cells in six-well plates were infected with rA2, rA2-PP2, (c)f rA2-PP5 at an (MOI) of 1.0 or 0.01. After 1 h of adsorption at room temperature, the infected cells were washed three times with phosphate-buffered saline (PBS), overlaid with 3 ml of Opti-MEM I (Invitrogen), and incubated at 35° C. At 24 h intervals, 200 μl of culture supernatant was collected and stored at −80° C. in the presence of SPG (0.2 M sucrose, 3.8 M KH₂PO₄, 7.2 M K₂HPO₄, 5.4 M monosodium glutamate) prior to virus titration. After each aliquot was removed, an equal amount of fresh medium was added to the cells. The virus titer was determined by plaque assay on Vero cells at 35° C.

Virus release analyses were performed with HEp-2 and Vero cells. Cells in six-well plates were infected with rA2, rA2-PP2, or rA2-PP5 at an (MOI) of 1.0. At each time point, the culture supertnatants were collected, and then the cell monolayers were washed twice with PBS and scraped in 1 ml of OptiMEM I.

Viruses associated with the infected cells were released by a one-time freeze thaw. Infectious virus present in the culture medium or in the infected cells was titrated by plaque assay on Vero cells.

### Replication of rA2-PP2 and rA2-PP5 in Mice and Cotton Rats

Virus replication in vivo was determined in respiratory pathogen-free BALB/c mice and cotton rats (Sigmodon hispidus) obtained from Harlan. Mice or cotton rats in groups of eight were inoculated intranasally under light methoxyflurane anesthesia with 0.1 ml of inoculum containing 10⁶ PFU of virus per animal. Four days postinoculation, the animals were sacrificed by CO₂ asphyxiation, and the lung tissues were harvested. The tissues were homogenized in OptiMEM I (Invitrogen), and the virus titer per gram of lung tissue was determined by plaque assay on Vero cells.

### Metabolic Labeling of Viral Proteins in Infected Cells

To examine phosphorylation of P protein in virus-infected cells, Vero cells were infected with rA2, rA2-PP2, of rA2-PP5 at an MOI of 1.0 in duplicate. After incubation at 35° C. for 10 h, the cells were incubated for 30 min in Dulbecco's MEM (DMEM) lacking either cysteine and methionine or phosphate. One set of samples was then incubated with [³⁵S]Cys and [³⁵S]Met (Amersham Biosciences) at 100 μCi/ml, an the other set was incubated with ³³Pi (ICN) at 100 μCi/ml for 4 h. The radiolabeled proteins were extracted by lysis of the cell monolayers in radioimmunoprecipitation assay (RIPA) buffer (10 mM Tris-HCl [pH 7.5], 150 mM NaCl, 5 mM EDTA, 1% Triton X-100, 1% sodium deoxycholate, 0.1% sodium dodecyl sulfate).

### Immunoprecipitation and Western Blotting

The radiolabeled polypeptides were immunoprecipitated either by polyclonal goat anti-RSV A2 antibodies of by a mixture of anti-P protein monoclonal antibodies (IP/021P/76P) at 4° C. overnight. The antibody-protein complex was precipitated by the addition of 30 μl of protein G-agarose beads (Invitrogen), incubated at 4° C. for 1 h, and washed three times with RIPA buffer containing 300 mM NaCl. The immunoprecipitated polypeptides were electrophoresed by sodium dodecyl sulfate-polyacrylamide gel electrophoresis (SDS-PAGE) (15% polyacrylamide) and detected by autoradiography. The N and P proteins detected on the autoradiographs were quantified by densitometry with a Molecular Dynamics densitometer by using ImageQuant 5.0 for Windows NT (Molecular Dynamics). For Western blotting, Vero cells were infected with each virus at an MOI of 1.0, and the cells were lysed in protein lysis buffer at 48 h postinfection. Detection of viral proteins in the blot by polyclonal anti-RSV antibody was performed as described by Lu et al. (2002) J. Virol. 76:2871-2880.

### Northern Blotting Analysis of Viral RNA Synthesis

To examine RSV RNA expression, Vero or HEp-2 cells were infected with rA2, rA2-PP2, or rA2-PP5 at an MOI of 1.0. The total cellular RNA was prepared at 48 h postinfection with a QIAamp viral RNA mini kit (Qiagen). Equal amounts of total RNA were separated on 1.2% agarose gels containing formaldehyde and transferred to nylon membranes (Amersham Pharmacia Biotech) with a Turboblotter apparatus (Schleicher & Schnell). The membranes were hybridized with RSV gene specific riboprobes labeled with DIG. The positive-sense F protein gene probe was used to detect viral genomic RNA, and the negative-sense P protein gene was used to detect viral mRNA. Hybridization of the membranes with riboprobes was performed at 0.5° C. Signals from the hybridized probes were detected by using a DIG-Luminescent Detection Kit (Roche Molecular Biochemicals) and visualized by exposure to BioMax film (Kodak).

### Results

### Generation of P Protein Phosphorylation Mutants

The five phosphorylation sites in P protein at serines 116, 117, and 119 (116/117/119 [central region])and 232 and 237 (232/237 [C terminal region]) are well conserved in the pneumoviruses. To examine the role of P protein phosphorylation in virus replication, the serine residues in these two clusters were mutagenized to remove their phosphorylation potential. The three serines in the central region were substituted for with leucine, arginine, and leucine, respectively (Mut1 [LRLSS]), or aspartic acid to mimic the negative charges of the phosphate groups (Mut2 [DDDSS]). The two serines in the C-terminal region were changed to either aspartic acid (Mut3 [SSSDD]) or alanine (Mut4 [SSSAA]). In addition, all five serines were changed to LRLAA (Mut5) or LRLDD (Mut6) to eliminate all of the major P protein phosphorylation sites. The positions of the substituted residues in each mutant are summarized in FIG. 14.

### In Vivo Functions of Phosphorylation-Defective P Protein

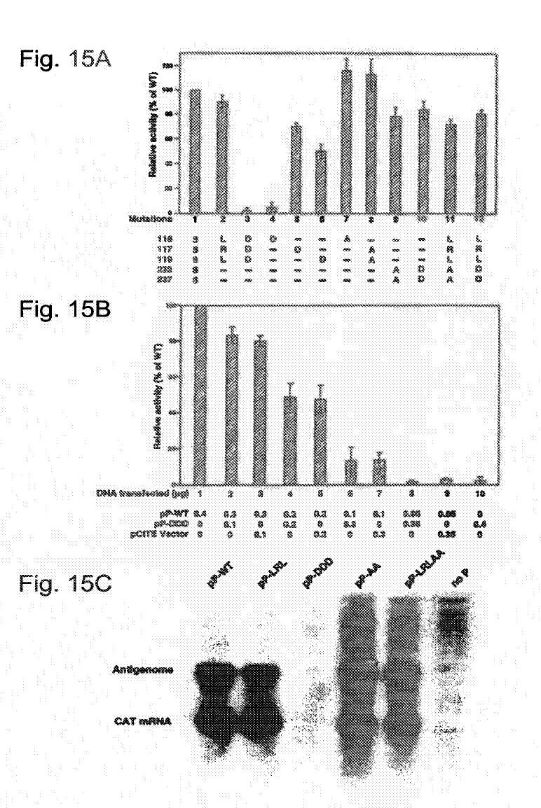

The functions of the altered P protein were evaluated in the RSV CAT minigenome assay. MVA-T7-infected HEp-2 cells were transfected with pRSVCAT along with pL, pN, and wild-type or-mutant pP, and expression of the CAT gene was measured by CAT-ELISA. The function of each P protein mutant was calculated as its relative activity compared to that of wild-type P protein. Error bars represent the standard deviation of three replicate experiments. As shown in FIG. 15A, substitution of the three central serines by LRL (lane 2) had little effect on protein function, but substitution of these three residues by aspartic acid (DDD, lane 3) almost completely abolished the protein's function. To evaluate each position independently, three single aspartic acid substitutions were made. As shown in FIG. 15A, S116D was not functional (lane 4), and the other two mutants (S117D, lane 5; S119D, lane 6) remained functional, albeit at a reduced level. However, substitution of Ser-116 or Ser-117/119 by alanine had no effect on P protein function in the minigenome assay. These observations indicated that the serines at 116/117/119 were not required for P protein function and that the aspartic acid residues might have a structural impact on the P protein. P protein mutation at the C-terminal phosphorylation sites, 232/237, substituted for by alanine (lane 9) or aspartic acid (lane 10), reduced the P protein function by approximately 10 to 20% (FIG. 15A). A slightly reduced level of reporter gene activity was detected in cells expressing mutant P protein that had all five serines removed (LRLAA, lane 11; LRLDD, lane 12). All of the P protein mutants expressed a level of P protein comparable to that of the wild-type in these assays as determined by Western blotting. Therefore, the minigenome assay indicated that removal of all five phosphorylation sites from RSV P protein did not have a significant impact on protein function in vitro. The difference in the protein activity among these P protein mutants could be due to the reduction of P protein phosphorylation or due to an alteration of P protein structure caused by substitutions of the phosphorylation sites.

Since Mut3 (DDDSS) almost completely abolished the P protein function, it was thus interesting to know if this mutant would exhibit any dominant-negative effect on the function of wild-type P protein. Plasmid pP-DDD was cotransfected with the wild-type P protein plasmid pP-wt in different ratios together with 0.4 μg of pN and 0.2 μg of pL to determine if this mutant would interfere with wild-type P protein function in the minigenome assay (FIG. 15B). The T7 expression vector (pCITE) was used as a control. The levels of reporter gene expression (expressed as a percentage of that of wild-type P protein) decreased in correlation with the decreased amount of wild-type pP, which was most likely due to suboptimal ratio among the N, P, and L proteins. However, pP-DDD reduced the reporter gene expression at a level similar to that of the pCITE vector control. Thus, it appeared Mut3 did not have any dominant-negative effect on wild-type P protein function.

Transcription and replication of the pRSVCAT/EGFP minigenome in cells expressing several P protein mutants were analyzed by Northern blotting analysis, pRSVCAT/EGFP was used in Northern blotting in order to better distinguish mRNA from antigenome or read-through RNA. The CAT mRNA and antigenomic RNA were not-detected in cells expressing pPDDD (FIG. 15C), confirming that this mutant P protein was not able to form functional polymerase. For pP-LRL, pP-AA, and pP-LRLAA, which were functional by the pRSVCAT minigenome assay, both CAT mRNA and antigenomic RNA were detected. However, it appeared that the amount of the antigenomic RNA was slightly lower for the P protein mutants containing substitutions of LRL residues.

### Replication of Recombinant Viruses rA2-PP2 and rA2-PP5 in Cell Culture

To examine the effect of P protein phosphorylation mutations on virus replication, two mutants were introduced into the RSV A2 antigenomic cDNA clone: one with mutations at the two C-terminal serines (SSSAA [PP2]) and the other with mutations at five serines (LRLAA [PE5]). Both recombinant viruses were obtained from the transfected cDNA and designated rA2-PP2 and rA2-PP5, respectively. Each virus was amplified in Vero cells, and both the released and cell-associated viruses were collected. rA2-PP2 and rA2-PP5 had titers of approximately 2×10⁷ PFU/ml in Vero cells, a level comparable to that of wild-type rA2.

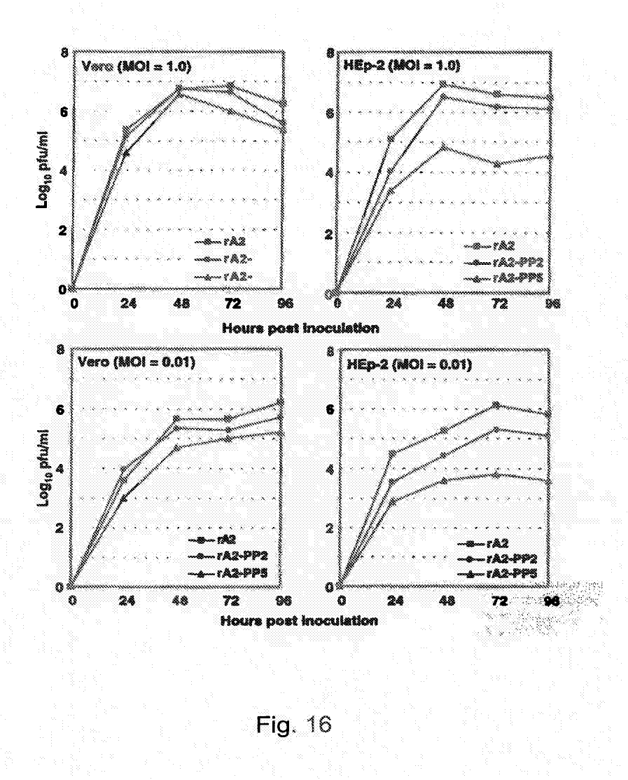

The single-cycle (MOI=1.0) and multicycle (MOI=0.01) replication kinetics of rA2-PP2 (circles) and rA2-PP5 (triangles) released into the culture medium were compared to that of rA2 (square) in both HEp-2 and Vero cells at 35° C. (FIG. 16). Aliquots of culture supernatant (200 μl) were harvested at 24-h intervals for 96 h. The virus titers are an average of two experiments. In Vero cells, both mutants reached peak titers slightly lower than that of wild-type rA2. In HEp-2 cells, however, rA2-PP2 and, to a greater extent, rA2-PP5 reached peak titers much lower than that of wild-type rA2. At an MOI of 1.0, the peak titer of rA2-PP2 was only slightly reduced (0.4 log₁₀), but rA2-PP5 had a peak titer reduction of 2.1 log₁₀. At an MOI of 0.01, the reductions in their peak titers were even greater: 0.8 log₁₀) for rA2-PP2 and 2.3 log₁₀ for rA2-PP5 (FIG. 16).

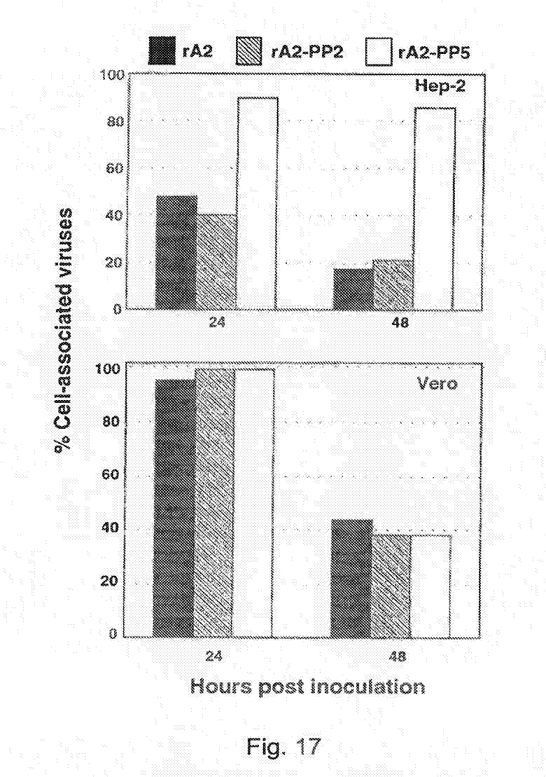

To investigate whether rA2-PP5 was inefficiently released from infected HEp-2 cells compared to Vero cells, HEp-2 or Vero cells were infected with rA2 (solid bars), rA2-PP2 (hatched bars), or rA2-PP5 (white bars), and the amount of virus released into the culture medium supernatant or associated with the cells was monitored by plaque assay (FIG. 17). In HEp-2 cells, at 24 h postinfection, less than 50% of rA2 and rA2-PP2 was associated with the cells. In contrast, approximately 90% of rA2-PP5 was associated with the cells. The percentages of cell-associated viruses for both rA2 and rA2PP5 at 48 h postinfection were decreased to around 20%. However, about 85% of rA2-PP5 remained cell associated (FIG. 17, upper panel). In contrast to the result obtained from the infected HEp-2 cells, rA2, rA2-PP2, and rA2-PP5 had a similar level of virus associated with the infected Vero cells. The majority of the viruses were cell associated at 24 h postinfection, and about 40% of the viruses remained cell associated at 48 h postinfection (FIG. 17, lower panel). These data demonstrated that dephosphorylation of P protein affected virus release from the infected HEp-2 cells, but not from the infected Vero cells.

### Phosphorylation of P Protein in rA2-PP2 and rA2-PP5 Infected Cells

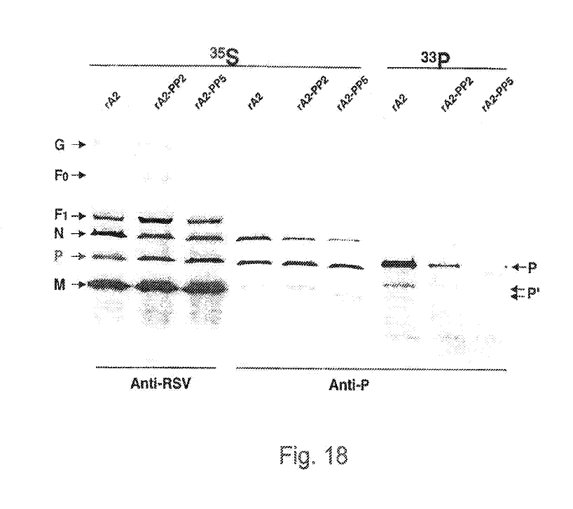

To examine the level of phosphorylation of P protein in infected cells, Vero cells were infected with rA2, rA2-PP2, or rA2-PP5 at an MOI of 1.0 and incubated at 35° C. At 18 h of postinfection, proteins were radiolabeled with [³⁵S]Cys and [^{35 S]Met (}100 μCi/ml) in DMEM deficient in cysteine and methionine or ³³Pi (100 μCi/ml) in DMEM deficient in phosphate for 4 h, immunoprecipitated either by anti-RSV polyclonal or by a mixture of anti-P protein monoclonal antibodies, separated by SDS-page (15% polyacrylamide), and autoradiographed (FIG. 18). P indicates the mature form of the F protein, and P′ represents the immature form of the P protein. The level of P protein expressed in rA2-PP2 and rA2-PP5-infected cells was comparable to that of wild-type rA2, as shown by immunoprecipitation of ³⁵S-labeled infected cells. It appeared that the migration pattern of the mature form of P protein was not significantly changed by the P protein phosphorylation status. In addition to the major P protein species that migrated at approximately 35 kDa, a faster-migrating protein band was also detected by anti-P protein antibodies, and the band of rA2-PP5 migrated even faster. Phosphorylation of P protein was reduced by about 80% for rA2-PP2 and 95% for rA2-PP5 compared to that of rA2. Only a trace amount of P protein labeled with [³³P]phosphate was detected in rA2-PP5-infected cells.

Anti-P monoclonal antibodies also immunoprecipitated the N protein in addition to P protein because of the specific N-P protein interaction in the infected cells. As shown in FIG. 18, the N protein immunoprecipitated by anti-P antibodies was reduced in rA2-PP2- and rA2-PP5-infected cells. The reduction of N protein was greater in rA2-PP5-infected cells (60%) than in rA2-PP2-infected cells (30%). Both rA2-PP2 and rA2-PP5 had an N/P protein ratio similar to that of wild-type rA2 when precipitated by anti-RSV antibodies. Thus, removal of the potential phosphorylation sites in P protein affected the interactions between the N and P proteins.

### Viral RNA and Protein Synthesis in rA2-PP2 and rA2-PP5 Infected cells

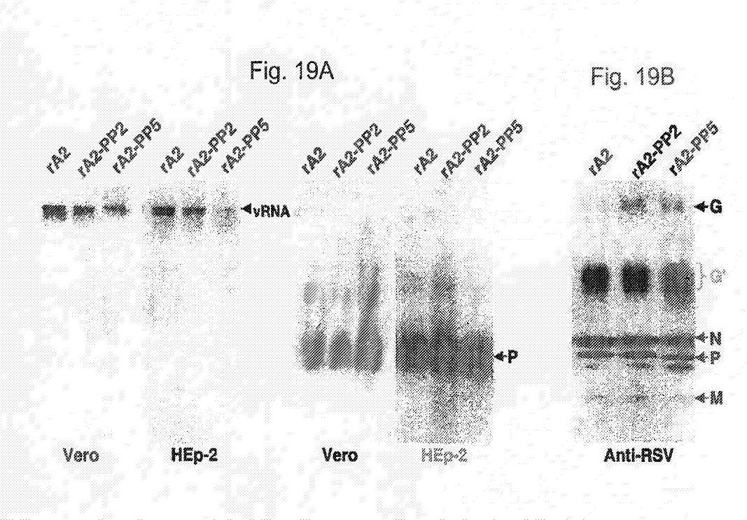

Synthesis of viral RNA and protein in rA2-PP2 and rA2-PP5-infected cells was evaluated by Northern and Western blotting analyses. Vero or HEp-2 cells were infected with wild-type rA2, rA2-PP2, and rA2-PP5 at an MOI of 1.0, and viral RNA was extracted 48 h postinfection. As shown in FIG. 19A, in the infected Vero cells, genomic RNA (vRNA) synthesis was slightly reduced for rA2-PF2 and more reduced for rA2-PP5. However, the P protein mRNA level was not reduced in rA2-PP5-infected cells. Instead, a slightly increased amount of mRNA was detected in rA2-PP5-infected cells. In the infected HEp-2 cells, rA2-PP5 also had a reduced ratio of genomic RNA to mRNA. Interestingly, the change in the genomic RNA/mRNA ratio was consistently observed throughout the course of infection only when an MOI of 1.0 was used. To examine whether viral protein synthesis was also increased in rA2-PP5-infected Vero cells, Western blotting was performed (FIG. 19B). Except for the slightly increased G protein synthesis (G′ represents the partially glycosylated forms of G protein), the levels of N, P, and M proteins were not increased in rA2-PP5-infected cells. Thus, the increased mRNA produced in rA2-PP5-infected cells did not result in a concomitant increase in protein expression.

### Genetic Stability of the P Protein Phosphorylation Mutations

To examine the genetic stability of the P protein phosphorylation mutations, rA2-PP2 and rA2-PP5 were passaged in Vero and HEp-2 cells in duplicate for five consecutive times. Consistent with the virus release experiment, infection took longer with each increased passage in HEp-2 cells for rA2-FP5, and a reduced number of virus progeny were released from the infected cells. Viral RNA was extracted from the infected cell culture supernatant at the 5th passage, and the P protein gene cDNA was obtained by RT-PCR and sequenced. All of the introduced mutations were maintained throughout the passages for both rA2-PP2 and rA2-PP5.

### Replication of rA2-PP2 and rA2-PP5 in Mice and Cotton Rats

Replication of rA2-PP2 and rA2-PF5 in the lower respiratory tracts of mice and cotton fats was examined (Table 5).

TABLE 5
Replication of recombinant RSV in mice and cotton rats.

Virus titer in lungs (mean
log₁₀ PFU/g ± SE)^{a}

| Virus | Mice | Cotton Rats |
|---|---|---|
| rA2 | 4.64 ± 0.08 | 4.72 ± 0.08 |
| rA2-PP2 | 2.80 ± 0.29 | 2.91 ± 0.29 |
| rA2-PP5 | 1.58 ± 1.06 | 1.61 ± 0.80 |

^{a}Groups of eight Balb/c mice or cotton rats were inoculated with 10⁶ PFU of virus intranasally under light anesthesia on day 0 and sacrificed on day 4. Virus titers per gram of lung tissue were determined by plaque assay.

Consistent with its growth kinetics in cell culture, rA2-PP5 was more attenuated in replication in the lower respiratory tracts of mice and cotton rats. The replication of rA2-PP2 and rA2-PP5 was reduced by 1.84 and 3.06 log₁₀, respectively, in the lungs of mice and by 1.81 and 3.11 log₁₀, respectively, in the lungs of cotton rats.

### Example 4

### Detection of Neutralizing Antibodies

### Materials and Methods

### Cells, Media And Viruses

Vero and HEp-2 cells obtained from American Type Culture Collection (ATCC, Rockville, Md.) were cultured in minimal essential medium (MEM) containing 5% fetal bovine serum (FBS). Wild-type subgroup A RSV A2 and subgroup B RSV 9320 strains were obtained from ATCC and grown in Vero cells using serum-free OptiMEM I (Invitrogen). Recombinant RSV A2 strain and rA2-G_{B}F_{B} have been described previously (Jin et al. (1998) Virology 251:206-214; Cheng et al. (2001) Virology 283:59-68) and grown in Vero cells. Modified vaccinia virus Ankara expressing bacteriophage T7 polymerase (MVA-T7) was provided by Br Bernard Moss and grown in CEK cells.

### Plasma and Sera

Plasma samples were obtained from healthy adults that were tested to be RSV seropositive by Western blotting. African green monkeys were infected with 10⁵ pfu of rA2 or 9320 RSV intranasally and challenged intranasally 4 weeks later with equal amount of homologous RSV. Monkey sera were collected 4 weeks after the primary infection and 2 weeks after the challenge infection (Cheng et al. (2001) Virology 283:59-68). Sera from a group of four monkeys were pooled and used in the neutralization assay. Prior to neutralization assay, all plasma and sera were heat inactivated at 56° C. for 30 min to remove any residual complement activity. Because both A and B strain RSV infection is endemic throughout the world, it is difficult to obtain human sera that are negative for RSV antibody or have been exposed to only a single RSV species. Thus, monkey sera collected from animals infected with wild-type A2 strain of subgroup A RSV or 9320 strain of subgroup 8 RSV were used to test the specificity and sensitivity of the newly developed microneutralization assay.

### Construction of Antigenomic RSV cDNA Expressing the LacZ Gene

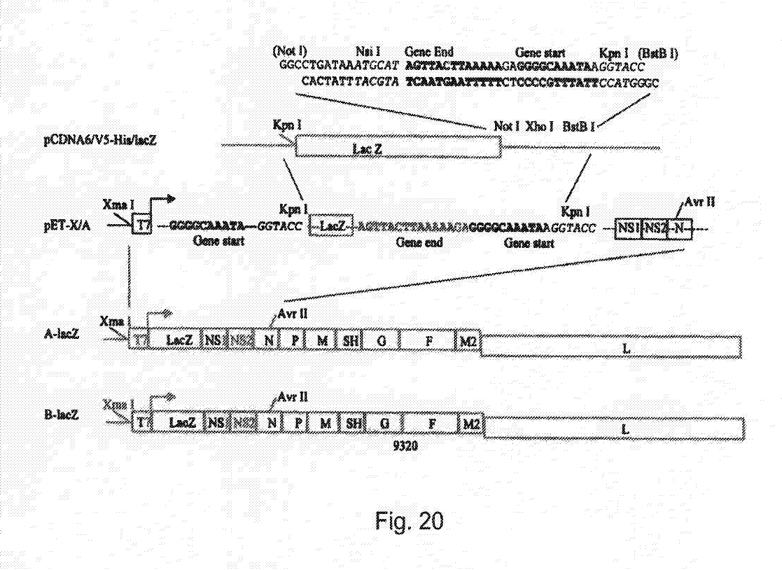

The construction of the recombinant RSV expressing the lacZ gene under the control of the RSV gene start (SEQ ID NO:6) and gene stop transcriptional signal is summarized in FIG. 20. A pair of the annealed oligonucleotides (upper, SEQ ID NO:7; lower, SEQ ID NO:8) containing the RSV gene end, gene start sequences and the Kpn I site was inserted downstream of the lacZ gene between the Not I and BstB I restriction sites of pcDNA/V5His/lacZ (Invitrogen). The plasmid was digested with Kpn I restriction enzyme and cloned into the Kpn I site of pRSV-X/A (pET-X/A), which contained the Xma I site, the T7 promoter, and RSV sequences from nt 1 to 2128 (Avr II). The Kpn I site was introduced at position of nt 93 between the NS1 gene start sequence and the NS I initiation site by QuikChange Site-Directed Mutagenesis Kit (Stratagene). The Xma I to Avr II fragment containing the inserted lacZ gene was then introduced into the RSV antigenomic cDNA done derived from A2 strain (pRSVC4G, Jin et al. (1998) Virology 251:206-214) and a chimeric RSV that had the G and F genes replaced by those of the subgroup B RSV 9320 strain (pA2-GbFb, Cheng et al. (2001) Virology 283:59-68). The antigenomic cDNA with the inserted lacZ gene in rRSVC4G and pA2-G_{B}F_{B} was designated, as pA-lacZ and pB-lacZ, respectively.

### Recovery of Recombinant RSV

Recovery of recombinant RSVs containing the lacZ gene, A-lacZ and B-lacZ, was performed as described previously (Jin et al. (1998) Virology 251:206-214). Briefly, HEp-2 cells were infected with MVA-T7 at an m.o.i. of 1 and transfected with 0.4 μg pN, 0.4 μg pP, 0.2 μg pL and 0.8 μg of pA-lacZ or pB-lacZ by LipofectACE (Invitrogen). Three days after transfection, the culture supernatant was used to infect the fresh Vero cells to amplify the recovered virus. The recombinant virus was then plaque purified and amplified in Veto cells. The virus titer was determined by plaque assay and the plaques were enumerated by immunostaining using polyclonal anti-RSV A2 serum (Biogenesis). The presence of the lacZ gene in the virus genome was confirmed by RT-PCR and expression of β-galactosidase was examined by staining of the infected cells with β-gal staining kit (Invitrogen).

### Replication of A-lacZ and B-lacZ in Tissue Culture

Replication of A-lacZ and B-lacZ in Vero and HEp-2 cells were, compared with rA2 and rA2G_{B}F_{B}. The cell monolayers in 6-well plate were infected with each virus in duplicate at an m.o.i of 0.3. After 1 h adsorption at room temperature, the infected cells were washed with PBS three times and incubated with 2 ml of OptiMEM at 35° C. At 24 h intervals, 250 μl of culture supernatant were removed and stored at −80° C. prior to virus titration. Each aliquot taken was replaced with the same amount of fresh media. The virus titer was determined by plaque assay on Vero cells.

### Egression of β-Galactosidase in Virus Infected Cells

The levels of β-galactosidase protein expressed by A-lacZ and B-lacZ were examined by Western blotting. Vero cells in 6-well plate were infected with virus at an m.o.i. of 0.05 and the total cell extracts were collected at 24 hour intervals for 7 days. The proteins were separated on 12% polyacrylamide gel containing SDS and transferred to a nylon membrane. The blot was blocked with 2% skim milk and incubated with a polyclonal antibody against β-galactosidase (Clontech) followed by incubation with an HRP-conjugated secondary antibody. The protein bands were detected by exposure to the X-ray film after detection with the ECL chemiluminescence detection kit (Amersham Pharmacia Biotech).

The β-galactosidase protein produced by A-lacZ and B-lacZ was also examined by its enzymatic activity. Vero cells in 96 well plates were infected with various amounts of A-lacZ or B-lacZ in triplicates and incubated at 35° C. from 1 to 5 days. After removal of the culture supernatant, the cell monolayers were washed twice with PBS and incubated in 200 μl of lysis buffer at 37° C. for 15 min. The lysis buffer contained 0.57 M Na₂HPO₄, 0.31 M NaH₂PO₄, 0.05 M KCl, 0.005 M MgSO₄, 0.1% NP-40, 20 mM β-mercaptoethanol and protease inhibitor cocktail (Roche Molecular Biochemicals) used at one tablet per 5 ml of the buffer. The plates were centrifuged at 2500 rpm for 5 min and 100 μl of the clarified lysates were transferred to fresh 96 well plates followed by the addition of 100 μl substrate solution containing 20 mM β-mercaptoethanol and 0.75 mM chlorophenol red β-D-galactopyranoside (CPRG, Roche Molecular Biochemicals) in phosphate buffer, pH 7.0. After incubation at 37° C. for 1-2 h, the optical density at a wavelength of 550 nm (OD550) was measured with SPECTRAmax, 340PC microplate spectrophotometer using SOFTmax software (Molecular Devices).

### Microneutralization Assay

Microneutralization assay was carried out in 96-well plates by the protocol described below. Heat-inactivated (56° C. for 30 min) serum or plasma samples were serially 2-fold diluted in 96-well plates in triplicate with OptiMEM/2% FBS or OptiMEM/2% FBS media containing 1:20 diluted guinea-pig complement (Invitrogen) in a final volume of 100 μl. A-lacZ or B-lacZ (approx. 150 pfu) in a volume of 50 μl was added to each well and incubated at 4° C. for 2 h. Approximately 50,000 Vero cells (50 μl) were then added to each well, and the plates were incubated at 35° C. for 3 days. The culture supernatant was removed, the cell monolayers were washed twice with PBS and incubated in 200 μl of lysis buffer at 37° C. for 15 min. The β-galactosidase enzymatic activity was then detected by incubation with the CPRG substrate as described above. The assay was shown to be responsive up to an OD550 of 3.0. Each test included control wells of uninfected cells, virus only, and positive serum control of known anti-RSV antibody titer. The mean anti-RSV neutralizing antibody titer was defined as the reciprocal log₂ of the highest antibody dilution that resulted in a 70% reduction in OD550 in comparison to un-neutralized virus infected control wells.

### Plaque Reduction Neutralization Assay

The plaque reduction neutralization assay (PRNT) was performed as previously described (Coates et al. (1966) Am. J. Epidemiol. 83:299-313) with some modifications. Two-fold serially diluted serum in 100 μl of volume was incubated with approx. 150 pfu of A2 in the presence of 1:20 diluted guinea pig complement or 150 pfu of A2 at 4° C. for 2 h. The antibody-virus mixtures were transferred to Vero cell monolayers in 12-well plates. After one hour adsorption at room temperature, the inocula were removed and the cell monolayers were overlayed with 1×L15 medium containing 1% methyl cellulose and 2% FBS. After incubation at 35° C. for 6 days, the plates were immunostained with a polyclonal anti-RSV serum. The plaques were counted and compared with the virus control wells that did not contain any antiserum. For each test, controls of virus only, uninfected cells, and positive control serum of known anti-RSV antibody titer were used to monitor the consistency of the assay. Anti-RSV neutralizing antibody titers were expressed as the reciprocal log₂ of the highest antibody dilution that had 50% reduction in plaque numbers compared to that of the un-neutralized virus infected control wells.

### Results

### Replication of Recombinant RSVs Expressing β-Galactosidase

To achieve a high level expression of β-galactosidase, the lacZ gene was inserted at the 3′ end of the RSV genome as the first gene expressed by RSV. Insertion of the foreign gene into this location was expected to have a minimal effect on the relative ratio of the downstream RSV gene expression, and thus was expected to have a minimal impact on virus replication. Recombinant RSVs containing the inserted lacZ gene, A-lacZ and B-lacZ, were recovered from HEp-2 cells, plaque purified and amplified in Vero cells.

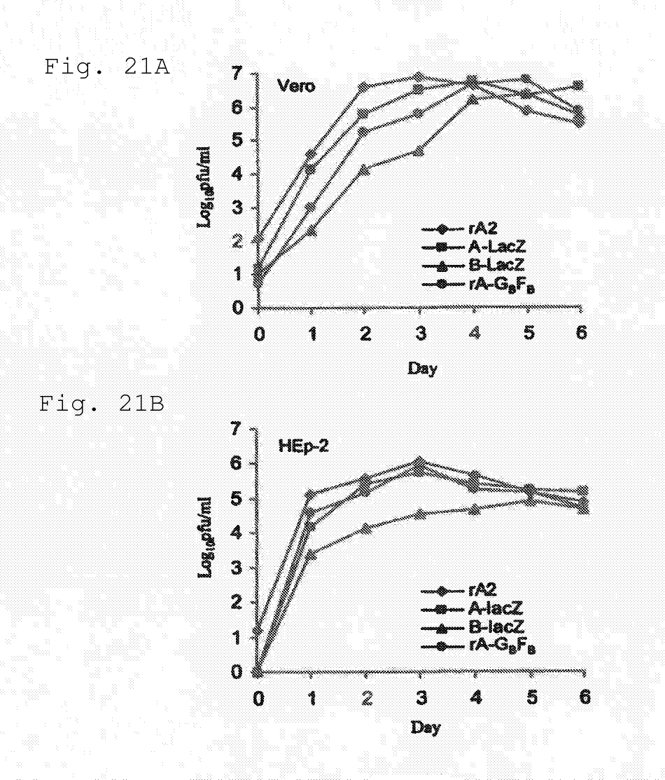

The impact of the 3.2 kb lacZ gene on virus replication was examined by multiple-step growth cycle analysis. Vero cells or HEp-2 cells were infected with recombinant RSV (A-lacZ, B-lacZ, rA2 or rA-G_{B}F_{B}) at an m.o.i of 0.3, and incubated at 35° C. for 7 days. Culture supernatants were collected daily for 6 days and titrated for virus amount by plaque assay on Vero cells. As shown in FIG. 21, growth of A-lacZ was slightly slower than rA2, but it eventually reached a peak titer similar to that of rA2, rA-G_{B}F_{B} replicated less well than rA2. Growth of 8-lacZ was even slower than A-lacZ and rA-G_{B}F_{B}. The titer of B-lacZ at the second and third days were more than 10-fold lower than rA-G_{B}F_{B}, but it reached a peak titer at day 5 that was within 2-fold of that of rA-G_{B}F_{B}.

In HEp-2 cells, A-lacZ grew similarly to that of rA2, but B-lacZ grew slower than rA-G_{B}F_{B}. The virus titer of B-lacZ at day 2 and day 3 was about 50-fold lower than rA-G_{B}F_{B}, but the reduction was less apparent at day 6. The reduced growth rates of A-lacZ and B-lacZ were likely due to the increased genome length resulting in an overall reduction of all the downstream protein expression. The inserted lacZ gene in the recombinant RSV was shown to be stable as examined by positive staining of β-galactosidase for majorities of virus plaques after ten passages in Vero cells.

### Expression of β-Galactosidase in Infected Cells

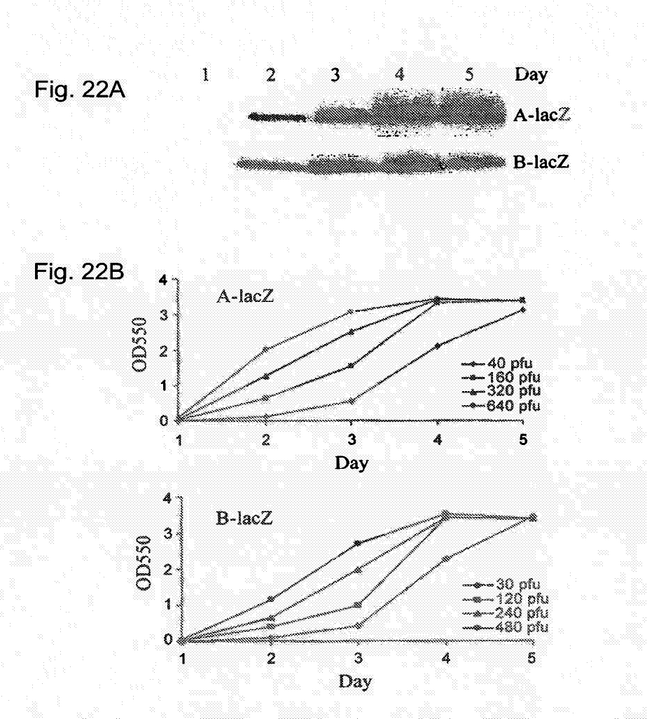

To examine the level of the β-galactosidase protein produced in the infected cells, Vero cells were infected with A-lacZ or B-lacZ at an m.o.i. of 0.05. The infected cells were collected every 24 hours and β-galactosidase was detected by Western blotting using anti-β-galactosidase antibody. As shown in FIG. 22A, β-galactosidase was produced in A-lacZ or B-lacZ infected cells at a level that was detected readily from the second day of infection and the protein level reached a peak on the fourth day of infection. Although B-lacZ did not replicate as efficiently as A-lacZ, it produced a level of β-galactosidase slightly higher than A-lacZ in the first 2 days of infection possibly because that B-lacZ was more cell-associated. β-galactosidase enzymatic activity was also detected in A-lacZ or B-lacZ infected Vero cells. Cells were infected with each virus at the amount indicated in FIG. 22B, the infected cells were collected daily and assayed for enzyme activity by incubating the cell lysate with CPRG in 96-well plates, and OD550 was determined by spectrophotometry. Measurement of the β-galactosidase enzyme activity using chlorophenol red-β-galactopyranoside (CPRG) as substrate also indicated that the enzyme activity saturated at the fourth day of infection when more than 120 pfu was used to infect Vero cells on 96 well plates (FIG. 22B). A linear response of enzyme activity was observed from the second to fourth days of the infection when approximately 150 pfu of A-lacZ or B-lacZ was used. Therefore, this amount of virus and an incubation time of 3 days were selected for the microneutralization assay.

### Microneutralization Assay Using β-Galactosidase Expressing RSVs

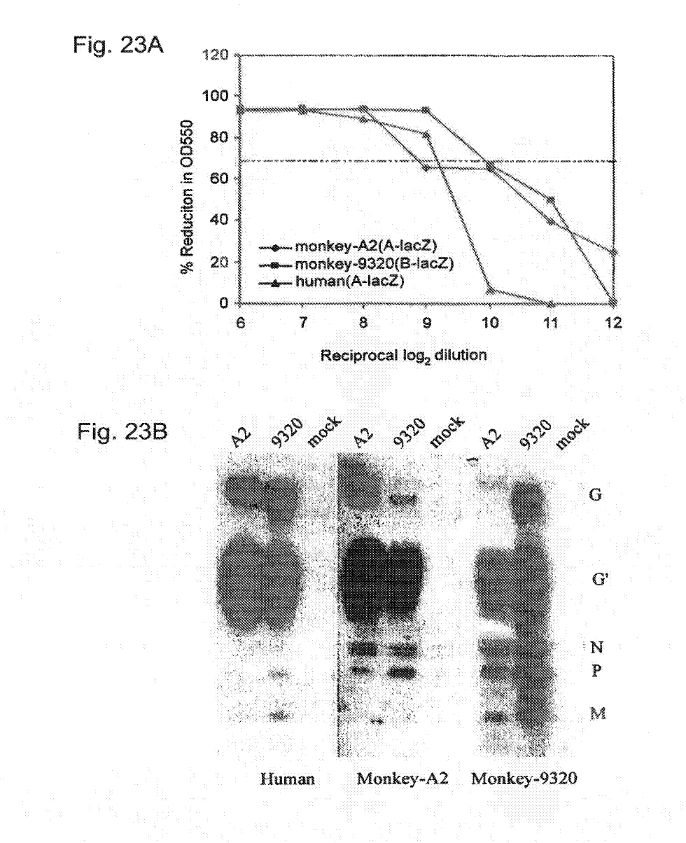

Since viral replication could be monitored by β-galactosidase activity, we determined whether viral neutralization could be measured using this marker. Serially 2-fold diluted adult human serum or sera collected from monkeys infected with RSV was incubated with 150 pfu of A-lacZ in the presence of 1:20 diluted complement or 150 pfu of B-lacZ for 2 h at 4° C. followed by the addition of the Vero cells in 96-well plates. After incubation at 35° C. for 3 days, the cells were lysed and the β-galactosidase activity was measured by spectrophotometry by monitoring the conversion of CPRG. The level of β-galactosidase was expressed as the percentage reduction in OD550 relative to the un-neutralized virus controls. As shown in FIG. 23A, a significant reduction in β-galactosidase activity was detected when the adult human serum was diluted up to 9.0 log₂. The calculated 70% reduction in OD550 was 9.0 log₂ for the human serum (triangles) tested and the reciprocal dilution of 9.0 log₂ was thus defined as the anti-RSV neutralizing antibody titer. When sera obtained from rA2 (diamonds) or 9320 (squares) RSV Infected monkeys were tested by this assay using A-lacZ or B-lacZ as neutralizing virus, a neutralizing antibody titer of 9.0 log₂ and 10.0 log₂ was determined respectively. As seen in FIG. 23A, variation in antibody titer was less apparent when the cutoff was defined at 70%. In addition, the antibody titer obtained by reduction in OD550 by 70% was more agreeable to the plaque reduction neutralization assay. Thus, the neutralizing antibody titer is defined as the highest reciprocal log₂ dilution of antiserum that had reduction in OB550 by 70 % compared to the un-neutralized virus control wells.

In order to test that the sera used in the microneutralization assay indeed contained antiRSV antibody, Western blot analysis was undertaken. Vero cells were infected with A2, 9320 RSV or mock-infected, the total cellular lysates 30 h after infection were separated on SDS-polyacrylamide gels, transferred to a nylon membrane and blotted with each serum as indicated in FIG. 23B. The human and monkey sera reacted mainly with the G protein that were fully (G) or partially (G′) glycosylated. Anti-F protein was not detectable by the Western blotting but detected by immunoprecipitation. Antibodies against several viral internal proteins (N, P, and M) were also detected. The human serum reacted well to the proteins of A2 and 9320, indicating that this individual was likely to have been exposed to both subgroup A and subgroup B RSV. Monkeys Infected with rA2 or 9320 RSV reacted with the homologous G protein much better than the heterologous G protein (FIG. 23B).

### Comparison of Microneutralization Assay with Plaque Reduction Assay

This microneutralization assay of the invention was compared with the plaque reduction neutralization assay as described by Coates et al. (1966) Am. J. Epidemiol. 83:299-313. RSV infected monkey sera samples of different levels of anti-RSV neutralizing antibody and a human adult serum containing a high level of anti-RSV antibody were used in the comparison. Each neutralization assay was performed with the homologous or heterologous RSV. Overall, the antibody levels measured by the microneutralization assay were comparable to the plaque reduction assay (Table 6).

TABLE 6
Comparison of levels of serum anti-RSV antibody detected by
two different assays

| | | Anti-RSV neutralizing antibody titer | | | |
|---|---|---|---|---|---|
| | | (mean reciprocal of log₂) | | | |
| | | Plaque | | | |
| | | reduction | | Microneutralization | |
| Serum | Infected | A2 | 9320 | A-lacZ | B-lacZ |
| Monkey | Pre- | 2.0 | 2.0 | 1.0 | 1.0 |
| | Infected | | | | |
| Monkey (4 w)^{a} | rA2 | 8.0 | 6.0 | 9.5 | 6.0 |
| Monkey (4 w) | 9320 | 6.0 | 9.0 | 9.7 | 10.3 |
| Monkey (6 w)^{b} | rA2 | 12.0 | 10.0 | 12.6 | 10.0 |
| Monkey (6 w) | 9320 | 9.0 | 11.0 | 10.3 | 12.3 |
| Monkey | NA^{c} | 9.0 | 8.0 | 9.0 | 9.0 |

^{a}Serum from four monkeys infected with RSV subgroup A recombinant A2(rA2) or subgroup B RSV 9320 strain were collected 4 weeks (4 w) after infection and pooled.
^{b}Monkeys were challenged with wt RSV A2 or 9320 at 4 weeks after infection and sera were collected 2 weeks later (6 w) and pooled.
^{c}Human adult serum was shown to contain antibodies against subgroup A and B RSV by Western blotting.
^{d}Plaque reduction neutralization assay or microneutralization assay were performed with homologous or heterologous virus as indicated. Anti-RSV serum neutralizing antibody titer was expressed as the mean reciprocal dilution of log₂. Except for microneutralization assay using the B-lacZ virus, all the others were performed in the presence of 1:20 diluted guinea pig complement.

For example, rA2 infected monkey sera collected 4 weeks post-infection had a titer of 9.5 log₂ as by microneutralization assay but had a titer of 8.0 log₂ as determined by the plaque reduction neutralization assay. A higher anti-RSV neutralizing antibody titer was detected in RSV infected monkey sera when the homologous virus was used in the neutralization assay than the heterologous virus in both assays, although significant cross reactivity was observed. As expected, anti-A2 antibody neutralized A-lacZ significantly better (2 log₂ higher) than B-lacZ in the microneutralization assay. However, the difference was less obvious when measuring anti-9320 antibody, only a slightly higher titer was detected with B-lacZ than A-lacZ for 9320 infected monkey sera collected 4 weeks after infection (Table 4). When plaque reduction neutralization assay was performed using the post-immune and post-challenge sera from monkeys infected with rA2 or 9320, a higher titer was also detected when homologous virus was used. This indicated that although there was a significant cross-reactivity between the two RSV subgroups, antigenic differences of the two subgroups could be distinguished by the neutralization assay. The human serum contained neutralizing antibody to A2 and 9320 at a similar level as determined by the two different neutralization assays.

### Example 5

### Function of the RSV M2-2 Protein

As noted, the M2-2 protein has been implicated in regulating RSV RNA replication and transcription in the virus life cycle. To further evaluate the role of M2-2 in replication and transcription, the effect of M2-2 overexpression on viral replication in cell culture and the effects of various mutations in M2-2 were examined.

### Overexpression of M2-2

The RSV A2 M2-2 transcriptional unit was amplified by PCR using primers containing the gene start or gene end sequence and appropriate restriction enzyme sites and was cloned either upstream of the NS1 gene (first position) or into the intergenic region between the F and M2 genes (eighth position). Moving the M2-2 ORF upstream of its normal position in the genome resulted in overexpression of M2-2, which resulted in genetically unstable viruses that acquired mutations decreasing M2-2 activity, M2-2 overexpression was thus not tolerated by RSV.

Table 7 summarizes the results of sequence analysis and analysis of the degree to which various M2-2 mutant proteins inhibited expression in a minigenome assay. M2-2G1 viruses have the M2-2 ORF at the first position of the genome, with a 3 nt (M2-2G1) or a 49 nt (M2-2G1-long) M2-2/NS-1 intergenic sequence. M2-2G8 viruses have the M2-2 ORF at the eighth position of the genome. As noted in the table, M2-2G8#A had acquired no mutations and had full function in the minigenome assay; however, in this virus, M2-2 protein expression is not significantly greater than in wild-type RSV.

TABLE 7
Analysis of inserted M2-2 sequence and in vitro function.

| | | M2-2 function |
|---|---|---|
| Virus | M2-2 Sequence | (% wt activity) |
| M2-2G8#A | NO changes | 100% |
| M2-2G8#B4 | 5 nts(1A/4T) insertion at 213 nt (72 aa)* | 33 |
| M2-2G8#B1 | 4 nts(4T) insertion at 213 nt (72 aa)* | 33 |
| M2-2G1#A2 | 1A insertion at 102 nt (34 aa) | ND |
| M2-2G1#A3 | IT insertion at 213 nt (71 aa)* | 33 |
| M2-2G1#B | Substitution and insertion at 123 nt* | ND |
| M2-2G1#C | T to A change at 17 nt (6aa) & | 66 |
| | substitution and insertion at 123 nt (41aa)* | |
| M2-2G1- | IA insertion at 12 nt (4aa) | ND |
| long | | |

*Indicates mixed sequence after the insertion site in different clones of the virus.
ND: not determined

### In Vitro Analysis of M2-2 Function

The M2-2 protein has been shown to be a strong inhibitor in the RSV minigenome system (Collins et al. (1996) Proc. Natl Acad Sci USA 93:81-85). Therefore, its function can be examined in this in vitro assay. To further characterize M2-2, the usage of M2-2 initiation codons and the impact of N-terminal and C-terminal truncations and amino acid substitutions on M2-2 in vitro function were examined.

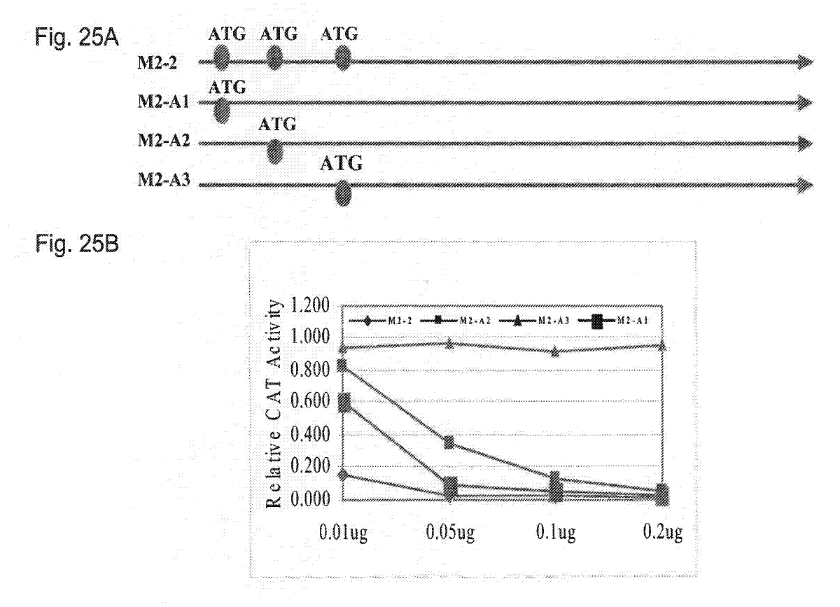

The M2-2 mRNA contains three AUG at its 5′ end. To determine whether all of these AUGs can be used to translate the M2-2 protein, two of the three AUG were removed by mutagenesis of the A2 M2-2 gene, and the protein translated from one of the three AUG was analyzed in vitro (FIG. 25). The protein translated from the first AUG (M2-A1) had an activity similar to that of wt M2-2 (having all three ATG). The protein translated from the second AUG (M2-A2) had slightly lower activity than M2-A1. The protein translated from the third AUG (M2-A3) did not function in vitro. Thus, this study indicated that either the first or the second AUG present in the M2-2 mRNA can be used to produce a functional M2-2 protein, and that forcing utilization of the second and/or third AUG can produce an M2-2 with decreased activity.

To further dissect the M2-2 protein structure and function, a series of deletion mutants were constructed. The protein was deleted either from the N-terminus or the C-terminus (Table 8). Truncation of as few as 6 amino acids from its N-terminus resulted in almost complete loss of M2-2 function. However, M2-2 truncation mutants with deletions from the C-terminus maintained partial function.

TABLE 8
Function of M2-2 deletion mutants in vitro.

| M2-2 deletion mutant | M2-2 function (% wt activity) |
|---|---|
| NΔ6 | 0.1 |
| NΔ8 | <0.1 |
| NΔ10 | <0.1 |
| CΔ1 | 52 |
| CΔ2 | 10 |
| CΔ4 | 19 |
| CΔ8 | 30 |
| CΔ18 | 33 |

A set of single and double amino acid substitutions were made in M2-2, and the mutant M2-2 proteins were tested for their in vitro inhibition activity. The M2-2 open reading frame was amplified by RT/PCR and cloned into a pCite2a/3a vector under the control of a T7 promoter. Amino acid substitution mutations in M2-2 were made in the M2-2 expression plasmid. Function of the expressed mutant M2-2 proteins was analyzed by a minigenome assay as described previously (Tang et al. (2001) J Virol 75:11328-11335). Briefly, HEp-2 cells were infected with MVA-T7 at an m.o.i of 1.0 and transfected with pL, pN, pP and a pRSVCAT minigenome together with various M2-2 mutant plasmids. Two days after transfection, the cell lysate was analyzed for the level of CAT protein. Wt M2-2 strongly inhibited RSV minigenome expression; the level of inhibition by each of the M2-2 mutants was expressed as relative activity compared to that of wt M2-2. As shown in Table 9, none of the single and double substitutions completely destroyed M2-2 function; mutation of Ile6 had the greatest effect.

TABLE 9
Analysis of M2-2 substitution mutations in vitro.

| aa substitution position | M2-2 function (% wt activity) |
|---|---|
| T2A | 97.6 |
| P4A | 91.4 |
| K5A | 95.5 |
| I6A | 73.7 |
| I6K | 66.5 |
| D11A | 98.9 |
| C15A | 97 |
| K12A | 96 |
| R25A, R27A | 97 |
| K34A | 95 |
| H47A | 97 |
| E56A, H58A | 95 |
| D66A | 98 |
| H75A | 98 |
| E80A, D81A | 82.7 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1-240. (canceled)

241. A recombinant respiratory syncytial virus (RSV) having an attenuated phenotype comprising a phosphoprotein (P), which phosphoprotein comprises at least one artificially mutated amino acid residue which eliminates a phosphorylation site wherein the phosphoprotein comprises an amino acid substitution selected from the group consisting of S116L, S116A, and S116D; an amino acid substitution selected from the group consisting of S117R, S117A, and S117D; an amino acid substitution selected from the group consisting of S119L, S119A, and S119D; an amino acid substitution selected from the group consisting of S232A and S232D; and an amino acid substitution selected from the group consisting of S237A and S237D, wherein the amino acid positions to be substituted are relative to the P protein of human RSV strain A2 as set forth in SEQ ID NO.:54 and FIG. 14.

242. The recombinant RSV of claim 241, wherein the recombinant RSV comprises a human RSV of subgroup A, subgroup B or a chimera thereof.

243. A nucleic acid encoding the recombinant RSV of claim 241.

244. The nucleic acid encoding the recombinant RSV of claim 242.

245. The nucleic acid of claim 243 wherein the nucleic acid is a DNA or RNA.

246. The nucleic acid of claim 244 wherein the nucleic acid is a DNA or RNA.

247. The nucleic acid of claim 245 wherein the nucleic acid is a RNA genome or antigenome.

248. The nucleic acid of claim 246 wherein the nucleic acid is a RNA genome or antigenome.

249. A vector comprising the nucleic acid of claim 243.

250. A vector comprising the nucleic acid of claim 244.

251. A live attenuated respiratory syncytial virus vaccine comprising an immunologically effective amount of a recombinant respiratory syncytial virus (RSV) having an attenuated phenotype comprising a phosphoprotein (P), which phosphoprotein comprises at least one artificially mutated amino acid residue which eliminates a phosphorylation site wherein the phosphoprotein comprises an amino acid substitution selected from the group consisting of S116L, S116A, and S116D; an amino acid substitution selected from the group consisting of S117R, S117A, and S117D; an amino acid substitution selected from the group consisting of S119L, S119A, and S119D; an amino acid substitution selected from the group consisting of S232A and S232D; and an amino acid substitution selected from the group consisting of S237A and S237D, wherein the amino acid positions to be substituted are relative to the P protein of human RSV strain A2 as set forth in SEQ ID NO.:54 and FIG. 14.

252. The vaccine of claim 250, further comprising a physiologically acceptable carrier.

253. The vaccine of claim 250, further comprising an adjuvant.

254. A method of stimulating the immune system of an individual to produce an immune response against RSV comprising administering to the individual, an immunologically effective amount of a recombinant respiratory syncytial virus (RSV) in a pharmaceutically acceptable carrier, having an attenuated phenotype comprising a phosphoprotein (P), which phosphoprotein comprises at least one artificially mutated amino acid residue which eliminates a phosphorylation site wherein the phosphoprotein comprises an amino acid substitution selected from the group consisting of S116L, S116A, and S116D; an amino acid substitution selected from the group consisting of S117R, S117A, and S117D; an amino acid substitution selected from the group consisting of S119L, S119A, and S119D; an amino acid substitution selected from the group consisting of S232A and S232D; and an amino acid substitution selected from the group consisting of S237A and S237D, wherein the amino acid positions to be substituted are relative to the P protein of human RSV strain A2 as set forth in SEQ ID NO.:54 and FIG. 14.

255. The method of claim 254, wherein the immune response is a protective immune response.

256. The method of claim 254, wherein the recombinant RSV is administered to the upper respiratory tract of the individual.

257. The method of claim 254, wherein the recombinant RSV is administered to the nasopharynx.

258. The method of claim 254, wherein the recombinant RSV is administered by spray, droplet or aerosol.

259. The method of claim 254 wherein the recombinant RSV comprises a human RSV of subgroup A, subgroup B or a chimera thereof.
